SEQUENCE LISTING (465 sequences)

SEQ ID NO: 1 - protein, 9 aa, Artificial sequence
  synthetic peptide
  MISC_FEATURE (1)..(1): N terminus myristlyated
RRHSTPHPD

SEQ ID NO: 2 - protein, 9 aa, Artificial sequence
  synthetic peptide
  MISC_FEATURE (1)..(1): N terminus myristlyated
  MISC_FEATURE (9)..(9): Amidated c terminus
RRHSTPHPD

SEQ ID NO: 3 - protein, 7 aa, Artificial sequence
  synthetic peptide
HSTPHPD

SEQ ID NO: 4 - protein, 10 aa, Artificial sequence
  synthetic peptide
  MISC_FEATURE (1)..(1): N terminus myristlyated
  MISC_FEATURE (10)..(10): DOTA conjugate
RRHSTPHPDK

SEQ ID NO: 5 - protein, 9 aa, Artificial sequence
  synthetic peptide
RRASTPHPD

SEQ ID NO: 6 - protein, 9 aa, Artificial sequence
  synthetic peptide
RRHATPHPD

SEQ ID NO: 7 - protein, 9 aa, Artificial sequence
  synthetic peptide
RRHSAPHPD

SEQ ID NO: 8 - protein, 9 aa, Artificial sequence
  synthetic peptide
RRHSTAHPD

SEQ ID NO: 9 - protein, 9 aa, Artificial sequence
  synthetic peptide
RRHSTPAPD

SEQ ID NO: 10 - protein, 9 aa, Artificial sequence
  synthetic peptide
RRHSTPHAD

SEQ ID NO: 11 - protein, 9 aa, Artificial sequence
  synthetic peptide
RRHSTPHPD

SEQ ID NO: 12 - protein, 9 aa, Artificial sequence
  synthetic peptide
RRHSTPHPA

SEQ ID NO: 13 - protein, 9 aa, Artificial sequence
  synthetic peptide
RRHSTPHPD

SEQ ID NO: 14 - protein, 10 aa, Artificial sequence
  synthetic peptide
RRHSTPHPDC

SEQ ID NO: 15 - protein, 10 aa, Artificial sequence
  synthetic peptide
RRHSTPHPDY

SEQ ID NO: 16 - protein, 10 aa, Artificial sequence
  synthetic peptide
RRHSTPHPDK

SEQ ID NO: 17 - protein, 10 aa, Artificial sequence
  synthetic peptide
  MISC_FEATURE (1)..(1): N terminus myristoylation
  MISC_FEATURE (10)..(10): Acetylated C terminus
RRHSTPHPDK

SEQ ID NO: 18 - protein, 9 aa, Artificial sequence
  synthetic peptide
RRHSTPHPD

SEQ ID NO: 19 - protein, 9 aa, Artificial sequence
  synthetic peptide
  MISC_FEATURE (1)..(1): N terminus myristoylation
  MISC_FEATURE (9)..(9): Biotin conjugate
RRHSTPHPD

SEQ ID NO: 20 - DNA, 21 bp, Artificial sequence
  Single strand DNA oligonucleotide
cccaagcaatggatgatttga

SEQ ID NO: 21 - DNA, 21 bp, Artificial sequence
  Single strand DNA oligonucleotide
ggcagaccagcatgacagatt

SEQ ID NO: 22 - DNA, 21 bp, Artificial sequence
  Single strand DNA oligonucleotide
gacctcaacgcacagtacgag

SEQ ID NO: 23 - DNA, 22 bp, Artificial sequence
  Single strand DNA oligonucleotide
aggcaaatgtgcaataccaaca

SEQ ID NO: 24 - DNA, 21 bp, Artificial sequence
  Single strand DNA oligonucleotide
actgtgacccttgcaccaaat

SEQ ID NO: 25 - DNA, 24 bp, Artificial sequence
  Single strand DNA oligonucleotide
aggcactcacagagcactacaaac

SEQ ID NO: 26 - DNA, 20 bp, Artificial sequence
  Single strand DNA oligonucleotide
acccactcctccacctttga

SEQ ID NO: 27 - DNA, 21 bp, Artificial sequence
  Single strand DNA oligonucleotide
gtggctctgattggctttctg

SEQ ID NO: 28 - DNA, 21 bp, Artificial sequence
  Single strand DNA oligonucleotide
gcgagactgtggccttgtgtc

SEQ ID NO: 29 - DNA, 23 bp, Artificial sequence
  Single strand DNA oligonucleotide
ggttgactcagcttttcctcttg

SEQ ID NO: 30 - DNA, 22 bp, Artificial sequence
  Single strand DNA oligonucleotide
ggataattagacgtacgtgggc

SEQ ID NO: 31 - DNA, 20 bp, Artificial sequence
  Single strand DNA oligonucleotide
gtattcccccaggtttacat

SEQ ID NO: 32 - DNA, 21 bp, Artificial sequence
  Single strand DNA oligonucleotide
ggcattctgggagcttcatct

SEQ ID NO: 33 - DNA, 20 bp, Artificial sequence
  Single strand DNA oligonucleotide
gcggattagggcttcctctt

SEQ ID NO: 34 - DNA, 22 bp, Artificial sequence
  Single strand DNA oligonucleotide
aggagtcccatgatgagattgt

SEQ ID NO: 35 - DNA, 26 bp, Artificial sequence
  Single strand DNA oligonucleotide
ggttacagcaccatcagtaggtacag

SEQ ID NO: 36 - DNA, 21 bp, Artificial sequence
  Single strand DNA oligonucleotide
gccaccccaagttagatctgg

SEQ ID NO: 37 - DNA, 18 bp, Artificial sequence
  Single strand DNA oligonucleotide
gcccttggacggcttttc

SEQ ID NO: 38 - DNA, 22 bp, Artificial sequence
  Single strand DNA oligonucleotide
ctgttgctgtagccaaattcgt

SEQ ID NO: 39 - DNA, 20 bp, Artificial sequence
  Single strand DNA oligonucleotide
cttgggctgcctgttttcag

SEQ ID NO: 40 - DNA, 20 bp, Artificial sequence
  Single strand DNA oligonucleotide
actttgtggaccctggaacg

SEQ ID NO: 41 - DNA, 21 bp, Artificial sequence
  Single strand DNA oligonucleotide
tatttaaaccatgcattttcc

SEQ ID NO: 42 - DNA, 23 bp, Artificial sequence
  Single strand DNA oligonucleotide
ggacaattgacaaaatcagtatc

SEQ ID NO: 43 - DNA, 21 bp, Artificial sequence
  Single strand DNA oligonucleotide
aggagtgagtggaagacagaa

SEQ ID NO: 44 - protein, 393 aa, homo sapiens [sequence omitted]

SEQ ID NO: 45 - protein, 199 aa, homo sapiens
SSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVHVCACPGRDRRTEEENLRKK

SEQ ID NO: 46 - protein, 6 aa, Artificial sequence
  synthetic peptide
HSAPHP

SEQ ID NO: 47 - RNA, 18 nt, Artificial sequence
  single guide RNA (sgRNA)
caccgccagcaaacgccg

SEQ ID NO: 48 - RNA, 19 nt, Artificial sequence
  single guide RNA (sgRNA)
aaccggacgaagaacaagg

SEQ ID NO: 49 - protein, 6 aa, Artificial sequence
  synthetic peptide
HSAPHP

SEQ ID NO: 50 - protein, 6 aa, Artificial sequence
  synthetic peptide
HSEPHP

SEQ ID NO: 51 - protein, 9 aa, Artificial sequence
  synthetic peptide
  MISC_FEATURE (1)..(1): N terminus myristoylation
  MISC_FEATURE (9)..(9): BSA conjugate
RRHSTPHPD

SEQ ID NO: 52 - protein, 9 aa, Artificial sequence
  synthetic peptide
  MISC_FEATURE (1)..(1): N terminus myristoylation
  MISC_FEATURE (9)..(9): KLH conjugate
RRHSTPHPD

SEQ ID NO: 53 - protein, 6 aa, Artificial sequence
  synthetic peptide
  MISC_FEATURE (1)..(1): selected from the group consisting of positively charged amino acid
  MISC_FEATURE (2)..(2): selected from the group consisting of Ser, Thr, Asn, Gln, Pro, Ala and Gly
  MISC_FEATURE (3)..(3): any amino acid
  MISC_FEATURE (4)..(4): selected from the group consisting of an alpha methyl amino and a beta-breaker amino acid
  MISC_FEATURE (5)..(5): selected from the group consisting of positively charged amino acid
  MISC_FEATURE (6)..(6): selected from the group consisting of an alpha methyl amino and a beta-breaker amino acid
XXXXXX

SEQ ID NO: 54 - protein, 6 aa, Artificial sequence
  synthetic peptide
  MISC_FEATURE (1)..(1): selected from the group consisting of His, Arg and Lys
  MISC_FEATURE (2)..(2): selected from the group consisting of Ser, Thr, Asn, Gln, Pro, Ala and Gly
  MISC_FEATURE (3)..(3): X is any amino acid
  misc_feature (4)..(4): Xaa can be any  amino acid
  MISC_FEATURE (5)..(5): selected from the group consisting of His, Arg and Lys
  misc_feature (6)..(6): Xaa can be any  amino acid
XXXXXX

SEQ ID NO: 55 - DNA, 47 bp, Artificial sequence
  Single strand DNA oligonucleotide
catgcccagacatgtccttgctgctgcgaacatgtcccaacatgttg

SEQ ID NO: 56 - DNA, 47 bp, Artificial sequence
  Single strand DNA oligonucleotide
caacatgttgggacatgttcgcagcagcaaggacatgtctgggcatg

SEQ ID NO: 57 - protein, 10 aa, Artificial sequence
  synthetic peptide
  MISC_FEATURE (1)..(1): N terminus myristoylation
  MISC_FEATURE (10)..(10): folate conjugate
RRHSTPHPDK

SEQ ID NO: 58 - protein, 15 aa, Artificial sequence
  synthetic peptide
  MISC_FEATURE (1)..(1): N terminus myristoylation
RRHSTPHPDHAYPHA

SEQ ID NO: 59 - protein, 21 aa, Artificial
  Peptide
  MISC_FEATURE (12)..(21): X= Arg or absent
IRILMFLIGCGXXXXXXXXXX

SEQ ID NO: 60 - protein, 28 aa, Artificial
  Peptide
  MISC_FEATURE (19)..(28): X=Arg or absent
LRCLLLLIGRVGRKKRRQXXXXXXXXXX

SEQ ID NO: 61 - protein, 17 aa, Artificial
  Peptide
  MISC_FEATURE (8)..(17): X=Arg or absent
YPTQGHLXXXXXXXXXX

SEQ ID NO: 62 - protein, 22 aa, Artificial
  Peptide
  MISC_FEATURE (13)..(22): X=Arg or absent
SWQALALYAAGWXXXXXXXXXX

SEQ ID NO: 63 - protein, 20 aa, Artificial
  Peptide
  MISC_FEATURE (11)..(20): X=Arg or absent
TLYLPHWHRHXXXXXXXXXX

SEQ ID NO: 64 - protein, 16 aa, Artificial
  Peptide
  MISC_FEATURE (7)..(16): X=Arg or absent
IRGRIIXXXXXXXXXX

SEQ ID NO: 65 - protein, 22 aa, Artificial
  Peptide
  MISC_FEATURE (13)..(22): X=Arg or absent
SFILFIRRGRLGXXXXXXXXXX

SEQ ID NO: 66 - protein, 22 aa, Artificial
  Peptide
  MISC_FEATURE (13)..(22): X=Arg or absent
HSSHHHPVHSWNXXXXXXXXXX

SEQ ID NO: 67 - protein, 17 aa, Artificial
  Peptide
  MISC_FEATURE (8)..(17): X=Arg or absent
HANLHHTXXXXXXXXXX

SEQ ID NO: 68 - protein, 20 aa, Artificial
  peptide
  MISC_FEATURE (11)..(20): X=Arg or absent
WNHHHSTPHPXXXXXXXXXX

SEQ ID NO: 69 - protein, 16 aa, Artificial
  Peptide
  MISC_FEATURE (7)..(16): X=Arg or absent
HSTPHPXXXXXXXXXX

SEQ ID NO: 70 - protein, 14 aa, Artificial
  Peptide
  MISC_FEATURE (5)..(14): X=Arg or absent
SILTXXXXXXXXXX

SEQ ID NO: 71 - protein, 20 aa, Artificial
  peptide
  MISC_FEATURE (11)..(20): X=Arg or absent
YRRLLIGMMWXXXXXXXXXX

SEQ ID NO: 72 - protein, 20 aa, Artificial
  Peptide
  MISC_FEATURE (11)..(20): X=Arg or absent
YRRLLIGMMWXXXXXXXXXX

SEQ ID NO: 73 - protein, 17 aa, Artificial
  Peptide
  MISC_FEATURE (8)..(17): X=Arg or absent
FPGHTIHXXXXXXXXXX

SEQ ID NO: 74 - protein, 22 aa, Artificial
  peptide
  MISC_FEATURE (1)..(10): X=Arg or absent
  MISC_FEATURE (1)..(22): D-amino_acid_peptide
XXXXXXXXXXGLRGRRIFLIFS

SEQ ID NO: 75 - protein, 12 aa, Artificial
  Peptide
LTFEHYWAQLTS

SEQ ID NO: 76 - protein, 12 aa, Artificial
  Peptide
GGGGGGGGGGGG

SEQ ID NO: 77 - protein, 12 aa, Artificial
  Peptide
NPNTYVPHWMRQ

SEQ ID NO: 78 - protein, 10 aa, Artificial
  peptide
YRRLLIGMMW

SEQ ID NO: 79 - protein, 13 aa, Artificial
  peptide
DEFHSFYTARQTG

SEQ ID NO: 80 - protein, 7 aa, Artificial
  Peptide
KPDSPRV

SEQ ID NO: 81 - protein, 12 aa, Artificial
  peptide
PPYSQFLQWYLS

SEQ ID NO: 82 - protein, 12 aa, Artificial
  peptide
SEFPRSWDMETN

SEQ ID NO: 83 - protein, 9 aa, Artificial
  peptide
HDTHNAHVG

SEQ ID NO: 84 - protein, 13 aa, Artificial
  peptide
WSEYDIPTPQIPP

SEQ ID NO: 85 - protein, 16 aa, Artificial
  peptide
SILTLSRRRRRRRRRR

SEQ ID NO: 86 - protein, 13 aa, Artificial
  peptide
SCRCRLRGDRGDR

SEQ ID NO: 87 - protein, 16 aa, Artificial
  peptide
GGGGGGGGGRRRRRRR

SEQ ID NO: 88 - protein, 12 aa, Artificial
  peptide
SEYLCSSLDAAG

SEQ ID NO: 89 - protein, 12 aa, Artificial
  peptide
GESFVQHVFRQN

SEQ ID NO: 90 - protein, 12 aa, Artificial
  peptide
SVHHHHRMHLVA

SEQ ID NO: 91 - protein, 7 aa, Artificial
  peptide
GRRRFCM

SEQ ID NO: 92 - protein, 7 aa, Artificial
  peptide
KLTIHHH

SEQ ID NO: 93 - protein, 7 aa, Artificial
  peptide
FGSHHEL

SEQ ID NO: 94 - protein, 7 aa, Artificial
  peptide
GTVDHHA

SEQ ID NO: 95 - protein, 10 aa, Artificial
  peptide
DRLSVFLFIM

SEQ ID NO: 96 - protein, 7 aa, Artificial
  peptide
AISHHTR

SEQ ID NO: 97 - protein, 12 aa, Artificial
  peptide
KHHPFDHRLGNQ

SEQ ID NO: 98 - protein, 7 aa, Artificial
  peptide
HSAHHTM

SEQ ID NO: 99 - protein, 7 aa, Artificial
  peptide
ELGLHRH

SEQ ID NO: 100 - protein, 7 aa, Artificial
  peptide
RRLRICV

SEQ ID NO: 101 - protein, 16 aa, Artificial
  peptide
VPHIHEFTRRRRRRRR

SEQ ID NO: 102 - protein, 5 aa, Artificial
  peptide
PLTLI

SEQ ID NO: 103 - protein, 5 aa, Artificial
  peptide
SLLIG

SEQ ID NO: 104 - protein, 5 aa, Artificial
  peptide
KPPER

SEQ ID NO: 105 - protein, 5 aa, Artificial
  peptide
CRIIR

SEQ ID NO: 106 - protein, 5 aa, Artificial
  peptide
SFILI

SEQ ID NO: 107 - protein, 5 aa, Artificial
  peptide
PHHHS

SEQ ID NO: 108 - protein, 4 aa, Artificial
  peptide
EFHS

SEQ ID NO: 109 - protein, 5 aa, Artificial
  peptide
RLRRL

SEQ ID NO: 110 - protein, 4 aa, Artificial
  peptide
DSPR

SEQ ID NO: 111 - protein, 5 aa, Artificial
  peptide
HPWTH

SEQ ID NO: 112 - protein, 5 aa, Artificial
  peptide
HFSHH

SEQ ID NO: 113 - protein, 4 aa, Artificial
  peptide
RRVI

SEQ ID NO: 114 - protein, 4 aa, Artificial
  peptide
ILVI

SEQ ID NO: 115 - protein, 16 aa, Artificial
  peptide
RRSRSNEDVEDKTEDE

SEQ ID NO: 116 - protein, 19 aa, Artificial
  peptide
RRIRSGGKDHAWTPLHENH

SEQ ID NO: 117 - protein, 18 aa, Artificial
  peptide
HTPHPPVARTSPLQTPRR

SEQ ID NO: 118 - protein, 13 aa, Artificial
  peptide
PDSEPPRMELRRR

SEQ ID NO: 119 - protein, 13 aa, Artificial
  peptide
RRDTFDIRILMAF

SEQ ID NO: 120 - protein, 15 aa, Artificial
  peptide
RREVTELHHTHEDRR

SEQ ID NO: 121 - protein, 12 aa, Artificial
  peptide
SPWTHERRCRQR

SEQ ID NO: 122 - protein, 15 aa, Artificial
  peptide
RSRSSHLRDHERTHT

SEQ ID NO: 123 - protein, 15 aa, Artificial
  peptide
RRRSTNTFLGEDFDQ

SEQ ID NO: 124 - protein, 14 aa, Artificial
  peptide
LIGLSTSPRPRIIR

SEQ ID NO: 125 - protein, 20 aa, Artificial
  peptide
EIYGESGKTDEHALDTEYRR

SEQ ID NO: 126 - protein, 18 aa, Artificial
  peptide
RRVILRSYDGGHSTPHPD

SEQ ID NO: 127 - protein, 18 aa, Artificial
  peptide
TGKTFVKRHLTEFEKKYR

SEQ ID NO: 128 - protein, 20 aa, Artificial
  peptide
NHFDYDTIELDTAGEYSRRR

SEQ ID NO: 129 - protein, 15 aa, Artificial
  peptide
DPEPPRYLPPPPERR

SEQ ID NO: 130 - protein, 20 aa, Artificial
  peptide
RRTFIRHRIDSTEVIYQDED

SEQ ID NO: 131 - protein, 17 aa, Artificial
  peptide
ESKTGHKSEEQRLRRYR

SEQ ID NO: 132 - protein, 17 aa, Artificial
  peptide
YDDEHNHHPHHSTHRRR

SEQ ID NO: 133 - protein, 18 aa, Artificial
  peptide
RRRREVHTIHQHGIVHSD

SEQ ID NO: 134 - protein, 11 aa, Artificial
  peptide
DEPLPPPERRR

SEQ ID NO: 135 - protein, 6 aa, Artificial
  peptide
SPHPPY

SEQ ID NO: 136 - protein, 19 aa, Artificial
  peptide
SPHPPYSPHPPYSPHPPYP

SEQ ID NO: 137 - protein, 9 aa, Artificial
  peptide
RRPHNLHHD

SEQ ID NO: 138 - protein, 12 aa, Artificial
  peptide
LRDPHPPERRIR

SEQ ID NO: 139 - protein, 15 aa, Artificial
  peptide
RRPADQISYLHPPER

SEQ ID NO: 140 - protein, 11 aa, Artificial
  peptide
DLQYDFPRIRR

SEQ ID NO: 141 - protein, 14 aa, Artificial
  peptide
YDELYQKEDPHRRR

SEQ ID NO: 142 - protein, 13 aa, Artificial
  peptide
FKPERFPQNDRRR

SEQ ID NO: 143 - protein, 8 aa, Artificial
  peptide
RPADRIRR

SEQ ID NO: 144 - protein, 11 aa, Artificial
  peptide
HDFDPRYRDRR

SEQ ID NO: 145 - protein, 14 aa, Artificial
  peptide
RIRRDPDSPLPHPE

SEQ ID NO: 146 - protein, 16 aa, Artificial
  peptide
  MYRISTATE (1)..(1): X=MYRISTATE
XRRIRILMFLIGCGRV

SEQ ID NO: 147 - protein, 14 aa, Artificial
  peptide
HPHVILPRIRIRIR

SEQ ID NO: 148 - protein, 12 aa, Artificial
  peptide
EIHTIHLLPERR

SEQ ID NO: 149 - protein, 13 aa, Artificial
  peptide
EPSHPRSRYPRTF

SEQ ID NO: 150 - protein, 16 aa, Artificial
  peptide
RNIIIRDFIHFSHIDR

SEQ ID NO: 151 - protein, 19 aa, Artificial
  peptide
  MYRISTATE (10)..(10): X=MYRISTATE
RRIRDPQIKXLEIHFSHID

SEQ ID NO: 152 - protein, 13 aa, Artificial
  peptide
  MYRISTATE (1)..(1): X=MYRISTATE
XDLHTIHIPRDRR

SEQ ID NO: 153 - protein, 14 aa, Artificial
  peptide
SHDFPHREPRPERR

SEQ ID NO: 154 - protein, 14 aa, Artificial
  peptide
SYRHYSDHWEDRRR

SEQ ID NO: 155 - protein, 20 aa, Artificial
  peptide
VWVHDSCHANLQNYRNYLLP

SEQ ID NO: 156 - protein, 20 aa, Artificial
  peptide
EHDFEVRGDVVNGRNHQGPK

SEQ ID NO: 157 - protein, 7 aa, Artificial
  peptide
LEVIYMI

SEQ ID NO: 158 - protein, 7 aa, Artificial
  peptide
WTLSNYL

SEQ ID NO: 159 - protein, 7 aa, Artificial
  peptide
DSLHSTY

SEQ ID NO: 160 - protein, 12 aa, Artificial
  peptide
WHHRQQIPRPLE

SEQ ID NO: 161 - protein, 12 aa, Artificial
  peptide
APSIFTPHAWRQ

SEQ ID NO: 162 - protein, 18 aa, Artificial
  peptide
THFSHHLKGGGRRQRRRP

SEQ ID NO: 163 - protein, 18 aa, Artificial
  peptide
LHSKTLVLGGGRRRRGDR

SEQ ID NO: 164 - protein, 19 aa, Artificial
  peptide
WTLSNYLGGRKKRRQRRRR

SEQ ID NO: 165 - protein, 12 aa, Artificial
  peptide
VRCIFRGIWVRL

SEQ ID NO: 166 - protein, 12 aa, Artificial
  peptide
HSSGHNFVLVRQ

SEQ ID NO: 167 - protein, 7 aa, Artificial
  peptide
LFILVFR

SEQ ID NO: 168 - protein, 7 aa, Artificial
  peptide
TTSHHPK

SEQ ID NO: 169 - protein, 12 aa, Artificial
  peptide
VMVLFRILRGSM

SEQ ID NO: 170 - protein, 4 aa, Artificial
  peptide
SILT

SEQ ID NO: 171 - protein, 17 aa, Artificial
  peptide
RRRESEQRSISLHHHST

SEQ ID NO: 172 - protein, 17 aa, Artificial
  peptide
  MYRISTATE (1)..(1): X=MYRISTATE
XHFNHYTFESTCRRRRC

SEQ ID NO: 173 - protein, 14 aa, Artificial
  peptide
HSTPHPPQPPERRR

SEQ ID NO: 174 - protein, 18 aa, Artificial
  peptide
RRKSEPHSLSGGYQTGAD

SEQ ID NO: 175 - protein, 21 aa, Artificial
  peptide
HRTGHYTRCRQRCRSRSHNRH

SEQ ID NO: 176 - protein, 14 aa, Artificial
  peptide
RRCRSILPLLLLSR

SEQ ID NO: 177 - protein, 21 aa, Artificial
  peptide
RTLHGRRVILHEGGHSISDLK

SEQ ID NO: 178 - protein, 18 aa, Artificial
  peptide
HHRLSYFIVRRHSTHASR

SEQ ID NO: 179 - protein, 10 aa, Artificial
  peptide
RRIRIDPQHD

SEQ ID NO: 180 - protein, 11 aa, Artificial
  peptide
ILQPDFLIRPE

SEQ ID NO: 181 - protein, 9 aa, Artificial
  peptide
HDPRIIRIR

SEQ ID NO: 182 - protein, 7 aa, Artificial
  peptide
SPYPIRT

SEQ ID NO: 183 - protein, 9 aa, Artificial
  peptide
ILVIIQRIM

SEQ ID NO: 184 - protein, 7 aa, Artificial
  peptide
IRFILIR

SEQ ID NO: 185 - protein, 7 aa, Artificial
  peptide
SSVHHRG

SEQ ID NO: 186 - protein, 7 aa, Artificial
  peptide
LRRQLQL

SEQ ID NO: 187 - protein, 7 aa, Artificial
  peptide
HTTAHTH

SEQ ID NO: 188 - protein, 12 aa, Artificial
  peptide
HPHNHTVHNVVY

SEQ ID NO: 189 - protein, 12 aa, Artificial
  peptide
DHSKFVPLFVRQ

SEQ ID NO: 190 - protein, 13 aa, Artificial
  peptide
SIRTLGRFLIIRV

SEQ ID NO: 191 - protein, 7 aa, Artificial
  peptide
GLCRIIL

SEQ ID NO: 192 - protein, 7 aa, Artificial
  peptide
SPPIRHH

SEQ ID NO: 193 - protein, 14 aa, Artificial
  peptide
HPTHPIRLRDNLTR

SEQ ID NO: 194 - protein, 13 aa, Artificial
  peptide
  MYRISTATE (1)..(1): X=MYRISTATE
XREEETILIIRRR

SEQ ID NO: 195 - protein, 18 aa, Artificial
  peptide
HTIHSISDFPEPPDRRRR

SEQ ID NO: 196 - protein, 20 aa, Artificial
  peptide
DEDAAHSTGHPHNSQHRRRR

SEQ ID NO: 197 - protein, 12 aa, Artificial
  peptide
TEQHHYIPHRRR

SEQ ID NO: 198 - protein, 12 aa, Artificial
  peptide
RLRRVILRSYHE

SEQ ID NO: 199 - protein, 19 aa, Artificial
  peptide
EEPDRQPSGKRGGRKRRSR

SEQ ID NO: 200 - protein, 13 aa, Artificial
  peptide
RDFHTIHPSISRR

SEQ ID NO: 201 - protein, 11 aa, Artificial
  peptide
RRVDIHDGQRR

SEQ ID NO: 202 - protein, 10 aa, Artificial
  peptide
DQPYPHRRIR

SEQ ID NO: 203 - protein, 14 aa, Artificial
  peptide
  MYRISTATE (1)..(1): X=MYRISTATE
XRDFILFIRRLGRR

SEQ ID NO: 204 - protein, 10 aa, Artificial
  peptide
LDLYHPRERR

SEQ ID NO: 205 - protein, 12 aa, Artificial
  peptide
RRIRDPLGNEHE

SEQ ID NO: 206 - protein, 8 aa, Artificial
  peptide
IVEFRIRR

SEQ ID NO: 207 - protein, 10 aa, Artificial
  peptide
RRPRIPDYIL

SEQ ID NO: 208 - protein, 12 aa, Artificial
  peptide
RSTPHIHEFIRR

SEQ ID NO: 209 - protein, 14 aa, Artificial
  peptide
SHDFYPHWMRERIR

SEQ ID NO: 210 - protein, 7 aa, Artificial
  peptide
HFSHHLK

SEQ ID NO: 211 - protein, 8 aa, Artificial
  peptide
TSPLQSLK

SEQ ID NO: 212 - protein, 13 aa, Artificial
  peptide
AILTLILRRVIWP

SEQ ID NO: 213 - protein, 7 aa, Artificial
  peptide
LRFIDYP

SEQ ID NO: 214 - protein, 10 aa, Artificial
  peptide
GPIKHHLQHH

SEQ ID NO: 215 - protein, 4 aa, Artificial
  peptide
LTLS

SEQ ID NO: 216 - protein, 20 aa, Artificial
  peptide
RYEENNGVNPPVQVFESRTR

SEQ ID NO: 217 - protein, 14 aa, Artificial
  peptide
REGFYGPWHEQRRR

SEQ ID NO: 218 - protein, 11 aa, Artificial
  peptide
RRDIIRHNAHS

SEQ ID NO: 219 - protein, 10 aa, Artificial
  peptide
HDFHDYLERR

SEQ ID NO: 220 - protein, 10 aa, Artificial
  peptide
IREFDPRRIR

SEQ ID NO: 221 - protein, 15 aa, Artificial
  peptide
RLRCLLLLIGRVGRR

SEQ ID NO: 222 - protein, 14 aa, Artificial
  peptide
LGIDEDEETETAPE

SEQ ID NO: 223 - protein, 12 aa, Artificial
  peptide
SLLIGFGIIRSR

SEQ ID NO: 224 - protein, 9 aa, Artificial
  peptide
VHEVTHHWL

SEQ ID NO: 225 - protein, 7 aa, Artificial
  peptide
ATPFHQT

SEQ ID NO: 226 - protein, 12 aa, Artificial
  peptide
SILPLFLIRRSG

SEQ ID NO: 227 - protein, 16 aa, Artificial
  peptide
SCRCRLRRRRRRRRRR

SEQ ID NO: 228 - protein, 7 aa, Artificial
  peptide
SRIVLGW

SEQ ID NO: 229 - protein, 7 aa, Artificial
  peptide
SNIHHQV

SEQ ID NO: 230 - protein, 10 aa, Artificial
  peptide
LTLMRLRIIG

SEQ ID NO: 231 - protein, 12 aa, Artificial
  peptide
HSYSPYYTFRQH

SEQ ID NO: 232 - protein, 5 aa, Artificial
  peptide
FILIR

SEQ ID NO: 233 - protein, 19 aa, Artificial
  peptide
RCRNRKKEKTECLQKESEK

SEQ ID NO: 234 - protein, 18 aa, Artificial
  peptide
RRIKMIRTSESFIQHIVS

SEQ ID NO: 235 - protein, 17 aa, Artificial
  peptide
RRVSELQRNKHGRKHEL

SEQ ID NO: 236 - protein, 19 aa, Artificial
  peptide
RRRLDDEDVQTPTPSEYQN

SEQ ID NO: 237 - protein, 14 aa, Artificial
  peptide
RRRQPLPSAPENEE

SEQ ID NO: 238 - protein, 20 aa, Artificial
  peptide
SPLQTPAAPGAAAGPALSPV

SEQ ID NO: 239 - protein, 10 aa, Artificial
  peptide
SHQVHTHHNN

SEQ ID NO: 240 - protein, 7 aa, Artificial
  peptide
KLQVPIK

SEQ ID NO: 241 - protein, 20 aa, Artificial
  peptide
IRGRIIRRKKRRQRRRRGDR

SEQ ID NO: 242 - protein, 12 aa, Artificial
  peptide
QIPHRSSTALQL

SEQ ID NO: 243 - protein, 7 aa, Artificial
  peptide
SYQTMQP

SEQ ID NO: 244 - protein, 18 aa, Artificial
  peptide
TDSHSHHRRRRRRRRRRR

SEQ ID NO: 245 - protein, 21 aa, Artificial
  peptide
IPMNFTSHSLRQRRRRRRRRR

SEQ ID NO: 246 - protein, 20 aa, Artificial
  peptide
YWSAPQPATRRRRRRRRRRR

SEQ ID NO: 247 - protein, 19 aa, Artificial
  peptide
STTHPHPGTSAPEPATRRR

SEQ ID NO: 248 - protein, 12 aa, Artificial
  peptide
DDSDNRIIRYRR

SEQ ID NO: 249 - protein, 15 aa, Artificial
  peptide
TSPHPSLPRHIYPRR

SEQ ID NO: 250 - protein, 20 aa, Artificial
  peptide
RRITEIRGRTGKTTLTYIED

SEQ ID NO: 251 - protein, 19 aa, Artificial
  peptide
  MYRISTATE (1)..(1): X=MYRISTATE
XDERTGKTRRYIDTRDIRR

SEQ ID NO: 252 - protein, 19 aa, Artificial
  peptide
  MYRISTATE (1)..(1): X=MYRISTATE
XMTYSDMPRRIITDEDRRR

SEQ ID NO: 253 - protein, 14 aa, Artificial
  peptide
RRYDTVIDDIEYRR

SEQ ID NO: 254 - protein, 11 aa, Artificial
  peptide
RDTIERPEIRR

SEQ ID NO: 255 - protein, 13 aa, Artificial
  peptide
  MYRISTATE (1)..(1): X=MYRISTATE
XRYRRLILEIWRR

SEQ ID NO: 256 - protein, 12 aa, Artificial
  peptide
  MYRISTATE (1)..(1): X=MYRISTATE
XRHDTHNAHIRR

SEQ ID NO: 257 - protein, 13 aa, Artificial
  peptide
THDFDRLLRIRRR

SEQ ID NO: 258 - protein, 10 aa, Artificial
  peptide
RHNHIRPDNQ

SEQ ID NO: 259 - protein, 11 aa, Artificial
  peptide
RYKEPRITPRE

SEQ ID NO: 260 - protein, 9 aa, Artificial
  peptide
LRIEPIRIR

SEQ ID NO: 261 - protein, 10 aa, Artificial
  peptide
  MYRISTATE (1)..(1): X=MYRISTATE
XRLIRIRILM

SEQ ID NO: 262 - protein, 14 aa, Artificial
  peptide
RPEFHSFHPIYERR

SEQ ID NO: 263 - protein, 7 aa, Artificial
  peptide
STTHIHA

SEQ ID NO: 264 - protein, 10 aa, Artificial
  peptide
FPHLVSSLTT

SEQ ID NO: 265 - protein, 12 aa, Artificial
  peptide
GLHLFTTDRQGW

SEQ ID NO: 266 - protein, 17 aa, Artificial
  peptide
NHPWQFPNRWTRRRRRR

SEQ ID NO: 267 - protein, 19 aa, Artificial
  peptide
HSSHHHPVHSWNRRRRRRR

SEQ ID NO: 268 - protein, 14 aa, Artificial
  peptide
  MYRISTATE (1)..(1): X=MYRISTATE
XDIHTIHLPDTHRR

SEQ ID NO: 269 - protein, 20 aa, Artificial
  peptide
VAEFAQSIQSRIVEWKERLD

SEQ ID NO: 270 - protein, 9 aa, Artificial
  peptide
TRILCIVMM

SEQ ID NO: 271 - protein, 12 aa, Artificial
  peptide
FLLPEPDENTRW

SEQ ID NO: 272 - protein, 7 aa, Artificial
  peptide
LMSNAQY

SEQ ID NO: 273 - protein, 15 aa, Artificial
  peptide
SILTLSCRCRLRLWR

SEQ ID NO: 274 - protein, 9 aa, Artificial
  peptide
HQIHRNHTY

SEQ ID NO: 275 - protein, 9 aa, Artificial
  peptide
LIRRCSLQR

SEQ ID NO: 276 - protein, 17 aa, Artificial
  peptide
GAMHLPWHMGTRRRRRR

SEQ ID NO: 277 - protein, 14 aa, Artificial
  peptide
DEDAKFRIRILMRR

SEQ ID NO: 278 - protein, 20 aa, Artificial
  peptide
NHITNGGEEDSDCSSRRRRL

SEQ ID NO: 279 - protein, 14 aa, Artificial
  peptide
  MYRISTATE (1)..(1): X=MYRISTATE
XHSSHHHPTVQHRR

SEQ ID NO: 280 - protein, 10 aa, Artificial
  peptide
RDFERTIVDI

SEQ ID NO: 281 - protein, 16 aa, Artificial
  peptide
  MYRISTATE (1)..(1): X=MYRISTATE
XRRREILHPEFRILYE

SEQ ID NO: 282 - protein, 9 aa, Artificial
  peptide
HHFSHHWKT

SEQ ID NO: 283 - protein, 7 aa, Artificial
  peptide
FLIRRSG

SEQ ID NO: 284 - protein, 12 aa, Artificial
  peptide
HNHHHSQHTPQH

SEQ ID NO: 285 - protein, 12 aa, Artificial
  peptide
HLHKHHYKDSRM

SEQ ID NO: 286 - protein, 17 aa, Artificial
  peptide
HRTQSTLILFIRRGRET

SEQ ID NO: 287 - protein, 9 aa, Artificial
  peptide
LHFSHIDRR

SEQ ID NO: 288 - protein, 10 aa, Artificial
  peptide
YELPHHAYPA

SEQ ID NO: 289 - protein, 19 aa, Artificial
  peptide
SLLIGFGIIRSRRRRRRRR

SEQ ID NO: 290 - protein, 17 aa, Artificial
  peptide
HTDSHPHHHHPHRRRRR

SEQ ID NO: 291 - protein, 19 aa, Artificial
  peptide
ATQHHYIKRRRRRRRRRRR

SEQ ID NO: 292 - protein, 19 aa, Artificial
  peptide
FRSFAIPLVVPFRRRRRRR

SEQ ID NO: 293 - protein, 19 aa, Artificial
  peptide
YPTQGHLRRRRRRRRRRRR

SEQ ID NO: 294 - protein, 18 aa, Artificial
  peptide
HANLHHTRRRRRRRRRRR

SEQ ID NO: 295 - protein, 20 aa, Artificial
  peptide
YRRLLIGMRRRRRRRRRRRR

SEQ ID NO: 296 - protein, 15 aa, Artificial
  peptide
SHYHTPQNPPSTRRR

SEQ ID NO: 297 - protein, 17 aa, Artificial
  peptide
RSYSKLLCLLERLRISP

SEQ ID NO: 298 - protein, 20 aa, Artificial
  peptide
FWTQSIKERKMLNEHDFEVR

SEQ ID NO: 299 - protein, 9 aa, Artificial
  peptide
THFSHHLKH

SEQ ID NO: 300 - protein, 7 aa, Artificial
  peptide
SCRCRLR

SEQ ID NO: 301 - protein, 18 aa, Artificial
  peptide
MHPPDWYHHTPKRRRRRR

SEQ ID NO: 302 - protein, 21 aa, Artificial
  peptide
HTIHVHYPGNRQPNPPLILQR

SEQ ID NO: 303 - protein, 14 aa, Artificial
  peptide
TPSYGHTPSHHRRR

SEQ ID NO: 304 - protein, 13 aa, Artificial
  peptide
  MYRISTATE (1)..(1): X=MYRISTATE
XIRGRIRIIRRIR

SEQ ID NO: 305 - protein, 11 aa, Artificial
  peptide
HHPWTHHQRWS

SEQ ID NO: 306 - protein, 12 aa, Artificial
  peptide
IPMNFTSHSLRQ

SEQ ID NO: 307 - protein, 12 aa, Artificial
  peptide
SNHHHRHHTNTH

SEQ ID NO: 308 - protein, 20 aa, Artificial
  peptide
EVTFRHSVVRRRRRRRRRRR

SEQ ID NO: 309 - protein, 18 aa, Artificial
  peptide
FPGHTIHRRRRRRRRRRR

SEQ ID NO: 310 - protein, 13 aa, Artificial
  peptide
SILTLSRIVLGWW

SEQ ID NO: 311 - protein, 10 aa, Artificial
  peptide
TLYLPHWHRH

SEQ ID NO: 312 - protein, 18 aa, Artificial
  peptide
SILTLRLRRLRRRRRRRR

SEQ ID NO: 313 - protein, 20 aa, Artificial
  peptide
TLYLPHWHRHRRRRRRRRRR

SEQ ID NO: 314 - protein, 7 aa, Artificial
  peptide
TDSHSHH

SEQ ID NO: 315 - protein, 20 aa, Artificial
  peptide
EWKERLDKEFSLSVYQKMKF

SEQ ID NO: 316 - protein, 7 aa, Artificial
  peptide
TIHPSIS

SEQ ID NO: 317 - protein, 12 aa, Artificial
  peptide
SILTLRLRRLRR

SEQ ID NO: 318 - protein, 8 aa, Artificial
  peptide
VPHIHEFT

SEQ ID NO: 319 - protein, 13 aa, Artificial
  peptide
TIIHREDEDEIEW

SEQ ID NO: 320 - protein, 12 aa, Artificial
  peptide
KDLPFYSHLSRQ

SEQ ID NO: 321 - protein, 19 aa, Artificial
  peptide
THFSHHLKHRRRRRRRRRR

SEQ ID NO: 322 - protein, 8 aa, Artificial
  peptide
ATQHHYIK

SEQ ID NO: 323 - protein, 12 aa, Artificial
  peptide
IIRGNFLIGGRL

SEQ ID NO: 324 - protein, 20 aa, Artificial
  peptide
LPNPPERHHRRRRRRRRRRR

SEQ ID NO: 325 - protein, 16 aa, Artificial
  peptide
SFILFIRRGRLGRGDR

SEQ ID NO: 326 - protein, 7 aa, Artificial
  peptide
FPGHTIH

SEQ ID NO: 327 - protein, 7 aa, Artificial
  peptide
CILRLWW

SEQ ID NO: 328 - protein, 16 aa, Artificial
  peptide
RRRSHSQENVDQDTDE

SEQ ID NO: 329 - protein, 18 aa, Artificial
  peptide
MSTESNMPRLIQNDDRRR

SEQ ID NO: 330 - protein, 7 aa, Artificial
  peptide
LLRLGLI

SEQ ID NO: 331 - protein, 12 aa, Artificial
  peptide
IRILMFLIGCGR

SEQ ID NO: 332 - protein, 8 aa, Artificial
  peptide
LHSKTLVL

SEQ ID NO: 333 - protein, 12 aa, Artificial
  peptide
LRCLLLLIGRVG

SEQ ID NO: 334 - protein, 12 aa, Artificial
  peptide
FLIGPDRLIRSR

SEQ ID NO: 335 - protein, 9 aa, Artificial
  peptide
LPNPPERHH

SEQ ID NO: 336 - protein, 12 aa, Artificial
  peptide
HTDSHPHHHHPH

SEQ ID NO: 337 - protein, 22 aa, Artificial
  peptide
  Fluorescein isothiocyanate (FITC) (1)..(1): X=Fluorescein isothiocyanate (FITC)
XSFILFIRRGRLGRRRRRRRRR

SEQ ID NO: 338 - protein, 12 aa, Artificial
  peptide
HSSHHHPVHSWN

SEQ ID NO: 339 - protein, 18 aa, Artificial
  peptide
  MYRISTATE (1)..(1): X=MYRISTATE
XRTLIGIIRSHHLTLIRR

SEQ ID NO: 340 - protein, 7 aa, Artificial
  peptide
IRGRIIR

SEQ ID NO: 341 - protein, 21 aa, Artificial
  peptide
IIRGNFLIGGRLRRRRRRRRR

SEQ ID NO: 342 - protein, 5 aa, Artificial
  peptide
IRILM

SEQ ID NO: 343 - protein, 12 aa, Artificial
  peptide
GAMHLPWHMGTL

SEQ ID NO: 344 - protein, 20 aa, Artificial
  peptide
KRGGRKRRGGGHRLSYFIRR

SEQ ID NO: 345 - protein, 12 aa, Artificial
  peptide
NHPWQFPNRWTV

SEQ ID NO: 346 - protein, 13 aa, Artificial
  peptide
MHPPDWYHHTPKH

SEQ ID NO: 347 - protein, 18 aa, Artificial
  peptide
SWQALALYAAGWRRRRRR

SEQ ID NO: 348 - protein, 4 aa, Artificial
  peptide
HNAH

SEQ ID NO: 349 - protein, 15 aa, Artificial
  peptide
DEFERYRRFSTSRRR

SEQ ID NO: 350 - protein, 9 aa, Artificial
  peptide
EVTFRHSVV

SEQ ID NO: 351 - protein, 20 aa, Artificial
  peptide
TRILCIVRKKRRQRRRRGDR

SEQ ID NO: 352 - protein, 18 aa, Artificial
  peptide
SILTLSRGRKKRRQRRRR

SEQ ID NO: 353 - protein, 18 aa, Artificial
  peptide
CILRLWWRRRRRRRRRRR

SEQ ID NO: 354 - protein, 13 aa, Artificial
  peptide
ASWQALALYAAGW

SEQ ID NO: 355 - protein, 17 aa, Artificial
  peptide
  MYRISTATE (1)..(1): X=MYRISTATE
XPRVLPSPHTIHPSQYP

SEQ ID NO: 356 - protein, 7 aa, Artificial
  peptide
HANLHHT

SEQ ID NO: 357 - protein, 22 aa, Artificial
  peptide
SFILFIRRGRLGRKKRRQRRRP

SEQ ID NO: 358 - protein, 8 aa, Artificial
  peptide
YPTQGHLR

SEQ ID NO: 359 - protein, 21 aa, Artificial
  peptide
YRRLLIGMMWRRRRRRRRRRR

SEQ ID NO: 360 - protein, 12 aa, Artificial
  peptide
SFILFIRRGRLG

SEQ ID NO: 361 - protein, 19 aa, Artificial
  peptide
IRILMFLIGCGRRRRRRRR

SEQ ID NO: 362 - protein, 14 aa, Artificial
  peptide
  MYRISTATE (1)..(1): X=MYRISTATE
XRRICRFIRICRVR

SEQ ID NO: 363 - protein, 16 aa, Artificial
  peptide
IRGRIIRRRRRRRRRR

SEQ ID NO: 364 - protein, 19 aa, Artificial
  peptide
RRRHDSCHNQLQNYDHSTE

SEQ ID NO: 365 - protein, 20 aa, Artificial
  peptide
WNHHHSTPHPRRRRRRRRRR

SEQ ID NO: 366 - protein, 19 aa, Artificial
  peptide
  MYRISTATE (1)..(1): X=MYRISTATE
XRRPVAPDLRHTIHIPPER

SEQ ID NO: 367 - protein, 12 aa, Artificial
  peptide
RRDIHTIHPFYQ

SEQ ID NO: 368 - protein, 12 aa, Artificial
  peptide
WNHHHSTPHPAH

SEQ ID NO: 369 - protein, 21 aa, Artificial
  peptide
SFILFIRRGRLGRRRRRRRRR

SEQ ID NO: 370 - protein, 20 aa, Artificial
  peptide
  D-amino_acid (1)..(20)
RRRRRRRRGLRGRRIFLIFS

SEQ ID NO: 371 - protein, 16 aa, Artificial
  peptide
  MYRISTATE (1)..(1): X=MYRISTATE
XRRHNAHHSTPHPDDR

SEQ ID NO: 372 - protein, 6 aa, Artificial
  peptide
HSTPHP

SEQ ID NO: 373 - protein, 20 aa, Artificial
  peptide
LRCLLLLIGRVGRKKRRQRR

SEQ ID NO: 374 - protein, 17 aa, Artificial
  peptide
  MYRISTATE (1)..(1): X=MYRISTATE
XRRKHNKHRPEPDSDER

SEQ ID NO: 375 - protein, 15 aa, Artificial
  peptide
  MYRISTATE (1)..(1): X=MYRISTATE
XRRIRDPRILLLHFD

SEQ ID NO: 376 - protein, 20 aa, Artificial
  peptide
RKRGKSYAFFVPPSESKERW

SEQ ID NO: 377 - protein, 15 aa, Artificial
  peptide
  MYRISTATE (1)..(1): X=MYRISTATE
XRRKILFIRLMHNKH

SEQ ID NO: 378 - protein, 17 aa, Artificial
  peptide
  MYRISTATE (1)..(1): X=MYRISTATE
XRRLIVRILKLPNPPER

SEQ ID NO: 379 - protein, 10 aa, Artificial
  peptide
  MYRISTATE (1)..(1): X=MYRISTATE
XRRHSTPHPD

SEQ ID NO: 380 - protein, 12 aa, Artificial
  peptide
KPPDRLWHYTQP

SEQ ID NO: 381 - protein, 12 aa, Artificial
  peptide
ATLPFVTDRQGW

SEQ ID NO: 382 - protein, 12 aa, Artificial
  peptide
FYSHSTSPAPAK

SEQ ID NO: 383 - protein, 12 aa, Artificial
  peptide
CYSHSYPTQGHL

SEQ ID NO: 384 - protein, 12 aa, Artificial
  peptide
EFHSFYTARQTG

SEQ ID NO: 385 - protein, 12 aa, Artificial
  peptide
SDGFVPHFKRQH

SEQ ID NO: 386 - protein, 7 aa, Artificial
  peptide
LPNPPER

SEQ ID NO: 387 - protein, 7 aa, Artificial
  peptide
LHSKTLV

SEQ ID NO: 388 - protein, 6 aa, Artificial
  peptide
HVHTHQ

SEQ ID NO: 389 - protein, 7 aa, Artificial
  peptide
SSSLGTH

SEQ ID NO: 390 - protein, 7 aa, Artificial
  peptide
HEVTHHW

SEQ ID NO: 391 - protein, 7 aa, Artificial
  peptide
SAPQPAT

SEQ ID NO: 392 - protein, 7 aa, Artificial
  peptide
TPPLTLI

SEQ ID NO: 393 - protein, 6 aa, Artificial
  peptide
HPWTHH

SEQ ID NO: 394 - protein, 7 aa, Artificial
  peptide
SAASDLR

SEQ ID NO: 395 - protein, 7 aa, Artificial
  peptide
SPLQSLK

SEQ ID NO: 396 - protein, 7 aa, Artificial
  peptide
RPTQVLH

SEQ ID NO: 397 - protein, 12 aa, Artificial
  peptide
FRSFAIPLVVPF

SEQ ID NO: 398 - protein, 12 aa, Artificial
  peptide
KILFIRLMHNKH

SEQ ID NO: 399 - protein, 4 aa, Artificial
  peptide
HHHP

SEQ ID NO: 400 - protein, 4 aa, Artificial
  peptide
HTIH

SEQ ID NO: 401 - protein, 4 aa, Artificial
  peptide
HNKH

SEQ ID NO: 402 - protein, 5 aa, Artificial
  peptide
LLLIG

SEQ ID NO: 403 - protein, 5 aa, Artificial
  peptide
ILFIR

SEQ ID NO: 404 - protein, 6 aa, Artificial
  peptide
IRGRII

SEQ ID NO: 405 - protein, 7 aa, Artificial
  peptide
SFILFIR

SEQ ID NO: 406 - protein, 7 aa, Artificial
  peptide
YPTQGHL

SEQ ID NO: 407 - protein, 10 aa, Artificial
  peptide
WNHHHSTPHP

SEQ ID NO: 408 - protein, 11 aa, Artificial
  peptide
IRILMFLIGCG

SEQ ID NO: 409 - protein, 12 aa, Artificial
  peptide
SWQALALYAAGW

SEQ ID NO: 410 - protein, 12 aa, Artificial
  peptide
  D-amino_acids (1)..(12)
GLRGRRIFLIFS

SEQ ID NO: 411 - protein, 18 aa, Artificial
  peptide
LRCLLLLIGRVGRKKRRQ

SEQ ID NO: 412 - protein, 10 aa, Artificial sequence
  pCAP 250 peptide variant
  LIPID (1)..(1): MYRISTATE group attached
  MISC_FEATURE (1)..(1): N terminus myristlyated
RRHSTPHPGE

SEQ ID NO: 413 - protein, 10 aa, Artificial sequence
  pCAP 250 peptide variant
  LIPID (1)..(1): MYRISTATE group attached
RRHSTPHPSE

SEQ ID NO: 414 - protein, 10 aa, Artificial sequence
  pCAP 250 peptide variant
  LIPID (1)..(1): MYRISTATE group attached
RRHSTPHPAD

SEQ ID NO: 415 - protein, 10 aa, Artificial sequence
  pCAP 250 peptide variant
  LIPID (1)..(1): MYRISTATE group attached
RRHSTPHPAE

SEQ ID NO: 416 - protein, 9 aa, Artificial sequence
  pCAP 250 peptide variant
  LIPID (1)..(1): MYRISTATE group attached
RRHSSPHPD

SEQ ID NO: 417 - protein, 9 aa, Artificial sequence
  pCAP 250 peptide variant
  LIPID (1)..(1): MYRISTATE group attached
RRHSVPHPD

SEQ ID NO: 418 - protein, 9 aa, Artificial sequence
  pCAP 250 peptide variant
  LIPID (1)..(1): MYRISTATE group attached
RRHSCPHPD

SEQ ID NO: 419 - protein, 9 aa, Artificial sequence
  pCAP 250 peptide variant
  LIPID (1)..(1): MYRISTATE group attached
  MISC_FEATURE (5)..(5): D Glutamic Acid
RRHSEPHPD

SEQ ID NO: 420 - protein, 9 aa, Artificial sequence
  pCAP 250 peptide variant
  LIPID (1)..(1): MYRISTATE group attached
  MISC_FEATURE (5)..(5): D Threonine
RRHSTPHPD

SEQ ID NO: 421 - protein, 9 aa, Artificial sequence
  pCAP 250 peptide variant
  LIPID (1)..(1): MYRISTATE group attached
  MISC_FEATURE (5)..(5): D Serine
RRHSSPHPD

SEQ ID NO: 422 - protein, 9 aa, Artificial sequence
  pCAP 250 peptide variant
  LIPID (1)..(1): MYRISTATE group attached
  MISC_FEATURE (5)..(5): D Valine
RRHSVPHPD

SEQ ID NO: 423 - protein, 9 aa, Artificial sequence
  pCAP 250 peptide variant
  LIPID (1)..(1): MYRISTATE group attached
  MISC_FEATURE (3)..(3): X is L- Diaminobutyric Acid (L-DAB)
RRXSTPHPD

SEQ ID NO: 424 - protein, 9 aa, Artificial sequence
  pCAP 250 peptide variant
  LIPID (1)..(1): MYRISTATE group attached
  misc_feature (7)..(7): X is L- Diaminobutyric Acid (L-DAB)
RRHSTPXPD

SEQ ID NO: 425 - protein, 10 aa, Artificial sequence
  pCAP 250 peptide variant
  LIPID (1)..(1): MYRISTATE group attached
  MISC_FEATURE (10)..(10): Methylated Amino Acid Residue
RRHSTPHPDD

SEQ ID NO: 426 - protein, 9 aa, Artificial sequence
  pCAP 250 peptide variant
  LIPID (1)..(1): MYRISTATE group attached
RRHSTPHAD

SEQ ID NO: 427 - protein, 9 aa, Artificial sequence
  pCAP 250 peptide variant
  LIPID (1)..(1): MYRISTATE group attached
RRHSKPHPD

SEQ ID NO: 428 - protein, 9 aa, Artificial sequence
  pCAP 250 peptide variant
  LIPID (1)..(1): MYRISTATE group attached
  misc_feature (7)..(7): X is L- Diaminobutyric Acid (L-DAB)
RRHSSPXPD

SEQ ID NO: 429 - protein, 9 aa, Artificial sequence
  pCAP 250 peptide variant
  LIPID (1)..(1): MYRISTATE group attached
  MISC_FEATURE (5)..(5): D Valine
  misc_feature (7)..(7): X is L- Diaminobutyric Acid (L-DAB)
RRHSVPXPD

SEQ ID NO: 430 - protein, 9 aa, Artificial sequence
  pCAP 250 peptide variant
  LIPID (1)..(1): MYRISTATE group attached
  misc_feature (7)..(7): X is L- Diaminobutyric Acid (L-DAB)
RRHSTPXAD

SEQ ID NO: 431 - protein, 9 aa, Artificial sequence
  pCAP 250 peptide variant
  LIPID (1)..(1): MYRISTATE group attached
  MISC_FEATURE (5)..(5): D Serine
  misc_feature (7)..(7): X is L- Diaminobutyric Acid (L-DAB)
RRHSSPXPD

SEQ ID NO: 432 - protein, 10 aa, Artificial sequence
  pCAP 250 peptide variant
  LIPID (1)..(1): MYRISTATE group attached
  MISC_FEATURE (10)..(10): C-Terminal Amidation
RRHSKPHPDD

SEQ ID NO: 433 - protein, 9 aa, Artificial sequence
  pCAP 250 peptide variant
  LIPID (1)..(1): MYRISTATE group attached
  misc_feature (3)..(3): X is L- Diaminobutyric Acid (L-DAB)
  misc_feature (7)..(7): X is L- Diaminobutyric Acid (L-DAB)
RRXSTPXPD

SEQ ID NO: 434 - protein, 9 aa, Artificial sequence
  pCAP 250 peptide variant
  LIPID (1)..(1): MYRISTATE group attached
  misc_feature (7)..(7): X is L- Diaminobutyric Acid (L-DAB)
RRHSKPXPD

SEQ ID NO: 435 - protein, 9 aa, Artificial sequence
  pCAP 250 peptide variant
  LIPID (1)..(1): MYRISTATE group attached
  misc_feature (3)..(3): X is L- Diaminobutyric Acid (L-DAB)
RRXSKPHPD

SEQ ID NO: 436 - protein, 9 aa, Artificial sequence
  pCAP 250 peptide variant
  LIPID (1)..(1): MYRISTATE group attached
  misc_feature (3)..(3): X is L- Diaminobutyric Acid (L-DAB)
  misc_feature (7)..(7): X is L- Diaminobutyric Acid (L-DAB)
RRXSKPXPD

SEQ ID NO: 437 - protein, 9 aa, Artificial sequence
  pCAP 250 peptide variant
  LIPID (1)..(1): MYRISTATE group attached
RRHSKPHAD

SEQ ID NO: 438 - protein, 10 aa, Artificial sequence
  pCAP 250 peptide variant
  LIPID (1)..(1): MYRISTATE group attached
RRHSKPHASE

SEQ ID NO: 439 - protein, 10 aa, Artificial sequence
  pCAP 250 peptide variant
  LIPID (1)..(1): MYRISTATE group attached
RRHSKPHPSE

SEQ ID NO: 440 - protein, 9 aa, Artificial sequence
  pCAP 250 peptide variant
  LIPID (1)..(1): MYRISTATE group attached
  misc_feature (3)..(3): X is L- Diaminobutyric Acid (L-DAB)
  MISC_FEATURE (5)..(5): D Serine
  misc_feature (7)..(7): X is L- Diaminobutyric Acid (L-DAB)
RRXSSPXPD

SEQ ID NO: 441 - protein, 9 aa, Artificial sequence
  pCAP 250 peptide variant
  LIPID (1)..(1): MYRISTATE group attached
  misc_feature (3)..(3): X is L- Diaminobutyric Acid (L-DAB)
  MISC_FEATURE (5)..(5): D Valine
  misc_feature (7)..(7): X is L- Diaminobutyric Acid (L-DAB)
RRXSVPXPD

SEQ ID NO: 442 - protein, 10 aa, Artificial sequence
  pCAP 250 peptide variant
  LIPID (1)..(1): MYRISTATE group attached
RRHSTPHASE

SEQ ID NO: 443 - protein, 9 aa, Artificial sequence
  pCAP 250 peptide variant
  LIPID (1)..(1): MYRISTATE group attached
  MISC_FEATURE (5)..(5): D Lysine
RRHSKPHPD

SEQ ID NO: 444 - protein, 9 aa, Artificial sequence
  pCAP 250 peptide variant
  LIPID (1)..(1): MYRISTATE group attached
  misc_feature (5)..(5): X is L- Diaminobutyric Acid (L-DAB)
RRHSXPHPD

SEQ ID NO: 445 - protein, 9 aa, Artificial sequence
  pCAP 250 peptide variant
  LIPID (1)..(1): MYRISTATE group attached
  misc_feature (5)..(5): X is L- Diaminobutyric Acid (L-DAB)
RRHSXPHAD

SEQ ID NO: 446 - protein, 9 aa, Artificial sequence
  pCAP 250 peptide variant
  LIPID (1)..(1): MYRISTATE group attached
  misc_feature (7)..(7): X is L- Diaminobutyric Acid (L-DAB)
RRHSEPXPD

SEQ ID NO: 447 - protein, 9 aa, Artificial sequence
  pCAP 250 peptide variant
  LIPID (1)..(1): MYRISTATE group attached
  misc_feature (3)..(3): X is L- Diaminobutyric Acid (L-DAB)
RRXSEPHPD

SEQ ID NO: 448 - protein, 9 aa, Artificial sequence
  pCAP 250 peptide variant
  LIPID (1)..(1): MYRISTATE group attached
  MISC_FEATURE (5)..(5): D Valine
  misc_feature (7)..(7): X is L- Diaminobutyric Acid (L-DAB)
  MISC_FEATURE (9)..(9): C-Terminal Amidation
RRHSVPXPD

SEQ ID NO: 449 - protein, 9 aa, Artificial sequence
  pCAP 250 peptide variant
  LIPID (1)..(1): MYRISTATE group attached
  MISC_FEATURE (6)..(6): X is 2-Aminoisobutyric acid ( Aib)
RRHSTXHAD

SEQ ID NO: 450 - protein, 13 aa, Artificial sequence
  pCAP 250 peptide variant
  LIPID (1)..(1): MYRISTATE group attached
RRHSTPHPDIEGR

SEQ ID NO: 451 - protein, 21 aa, Artificial sequence
  pCAP 250 peptide variant
  LIPID (1)..(1): MYRISTATE group attached
RRHSTPHPDIEGRGWQRPSSW

SEQ ID NO: 452 - protein, 9 aa, Artificial sequence
  pCAP 250 peptide variant
  LIPID (1)..(1): MYRISTATE group attached
  misc_feature (3)..(3): X is L- Diaminobutyric Acid (L-DAB)
  misc_feature (7)..(7): X is L- Diaminobutyric Acid (L-DAB)
RRXSEPXPD

SEQ ID NO: 453 - protein, 9 aa, Artificial sequence
  pCAP 250 peptide variant
  LIPID (1)..(1): MYRISTATE group attached
  misc_feature (7)..(7): X is L- Diaminobutyric Acid (L-DAB)
  MISC_FEATURE (9)..(9): C-Terminal Amidation
RRHSEPXPD

SEQ ID NO: 454 - protein, 9 aa, Artificial sequence
  pCAP 250 peptide variant
  LIPID (1)..(1): MYRISTATE group attached
  misc_feature (3)..(3): X is L- Diaminobutyric Acid (L-DAB)
RRXSEPHPD

SEQ ID NO: 455 - protein, 9 aa, Artificial sequence
  pCAP 250 peptide variant
  LIPID (1)..(1): MYRISTATE group attached
  MISC_FEATURE (5)..(5): L-Phosphoserine
  misc_feature (7)..(7): X is L- Diaminobutyric Acid (L-DAB)
RRHSSPXPD

SEQ ID NO: 456 - protein, 9 aa, Artificial sequence
  pCAP 250 peptide variant
  LIPID (1)..(1): MYRISTATE group attached
  MISC_FEATURE (5)..(5): D-Phosphoserine
  misc_feature (7)..(7): X is L- Diaminobutyric Acid (L-DAB)
RRHSSPXPD

SEQ ID NO: 457 - protein, 9 aa, Artificial sequence
  pCAP 250 peptide variant
  LIPID (1)..(1): MYRISTATE group attached
  MISC_FEATURE (5)..(5): L-Phosphoserine
  misc_feature (7)..(7): X is L- Diaminobutyric Acid (L-DAB)
  MISC_FEATURE (9)..(9): C-Terminal Amidation
RRHSSPXPD

SEQ ID NO: 458 - protein, 9 aa, Artificial sequence
  pCAP 250 peptide variant
  LIPID (1)..(1): MYRISTATE group attached
  misc_feature (7)..(7): X is L- Diaminobutyric Acid (L-DAB)
RRHSKPXPD

SEQ ID NO: 459 - protein, 9 aa, Artificial sequence
  pCAP 250 peptide variant
  LIPID (1)..(1): MYRISTATE group attached
  misc_feature (3)..(3): X is L- Diaminobutyric Acid (L-DAB)
RRXSKPHPD

SEQ ID NO: 460 - protein, 10 aa, Artificial sequence
  pCAP 250 peptide variant
  LIPID (1)..(1): MYRISTATE group attached
RRHSTPHPAH

SEQ ID NO: 461 - protein, 10 aa, Artificial sequence
  pCAP 250 peptide variant
  LIPID (1)..(1): MYRISTATE group attached
  misc_feature (10)..(10): X is L- Diaminobutyric Acid (L-DAB)
RRHSTPHPAX

SEQ ID NO: 462 - protein, 10 aa, Artificial sequence
  pCAP 250 peptide variant
  LIPID (1)..(1): MYRISTATE group attached
RRHSTPHPDH

SEQ ID NO: 463 - protein, 10 aa, Artificial sequence
  pCAP 250 peptide variant
  LIPID (1)..(1): MYRISTATE group attached
  MISC_FEATURE (5)..(5): D-Valine
  misc_feature (7)..(7): X is L- Diaminobutyric Acid (L-DAB)
RRHSVPXPDH

SEQ ID NO: 464 - protein, 10 aa, Artificial sequence
  pCAP 250 peptide variant
  LIPID (1)..(1): MYRISTATE group attached
RRHSTPHADH

SEQ ID NO: 465 - protein, 9 aa, Artificial sequence
  pCAP 250 peptide variant
  misc_feature (7)..(7): X is  Diaminobutyric Acid (DAB)
RRHSTPXPD

(19) United States
(12) Patent Application Publication
(10) Pub. No.: US 2022/0251159 A1
(43) Pub. Date: Aug. 11, 2022

(54) PEPTIDES AND USE OF SAME IN THE TREATMENT OF DISEASES, DISRODERS OR CONDITIONS ASSOCIATED WITH A MUTANT P53

(71) Applicant: YEDA RESEARCH AND DEVELOPMENT CO., LTD, Rehovot (IL)

(72) Inventors: Varda ROTTER, Rehovot (IL); Moshe OREN, Rehovot (IL); Perry TAL, Rehovot (IL); Shay EIZENBERGER, Rehovot (IL); Avi BEN-SHIMON, Rehovot (IL)

(73) Assignee: YEDA RESEARCH AND DEVELOPMENT CO., LTD, Rehovot (IL)

(21) Appl. No.: 17/549,369

(22) Filed: Dec. 13, 2021

Related U.S. Application Data
(63) Continuation-in-part of application No. 16/074,086, filed on Jul. 31, 2018, now Pat. No. 11,230,578.
(60) Provisional application No. 62/291,003, filed on Feb. 4, 2016.

(51) Int. Cl. C07K 14/47; A61P 35/00; C12N 15/09

(52) U.S. Cl. CPC C07K 14/4746; C12N 15/09; A61P 35/00

(57) ABSTRACT

An isolated peptide is provided. The peptide comprises an amino acid sequence arranged in a space and configuration that allow interaction of the peptide with the DNA Binding Domain (DBD) of p53 through at least one residue of the DBD by which pCAP 250 (SEQ ID NO: 1) binds the DBD, wherein the peptide at least partially reactivates a mutant p53 protein, with the proviso that the peptide is not SEQ ID NO: 59-382.

### CROSS-REFERENCE TO RELATED APPLICATIONS

This application is a continuation application of U.S. application Ser. No. 16/074,086, filed on Jul. 31, 2018, which is a 371 application of International Application No. PCT/IL2017/050132 filed on Feb. 3, 2017, which claims priority to U.S. Provisional Application No. 62/291,003, filed Feb. 4, 2016.

### SEQUENCE LISTING

The Sequence Listing submitted herewith is an ASCII text file (2021-12-13 Sequence Listing.text, created on Dec. 13, 2021, 4828788 bytes) via EFS-Web is hereby incorporated by reference.

### FIELD AND BACKGROUND OF THE INVENTION

The present invention, in some embodiments thereof, relates to peptides and use of same in the treatment of diseases, disorders or conditions associated with a mutant p53.

Cancer is a leading cause of death in developed countries, and as the average age of the population continues to rise, so do the numbers of diagnosed cases and economic implications. Cancer is not a single disease, but rather a group of more than 200 diseases characterized by uncontrolled growth and spread of abnormal cells. Cancer is a highly heterogeneous disease with major molecular differences in the expression and distribution of tumor cell surface markers even among patients with the same type and grade of cancer. Moreover, cellular mutations tend to accumulate as cancer progresses, further increasing tumor heterogeneity. Most tumor cells exhibit genomic instability with an increased expression of oncogenes and inactivation of tumor suppressor genes.

The p53 gene is considered to be the most important tumor suppressor gene that acts as a major barrier against cancer progression. The p53 protein responds to various types of cellular stress, and triggers cell cycle arrest, apoptosis, or senescence. This is achieved by transcriptional transactivation of specific target genes carrying p53 DNA binding motifs. It is widely agreed that the p53 pathway is impaired in almost all human cancers. Mutation of p53 is viewed as a critical step in malignant transformation process and over 50% of cancer cases carry mutations in their p53 genes. Most of these mutations are missense point mutations that target the DNA-binding core domain (DBD) of p53, thereby abolishing specific DNA binding of p53 to its target site. These mutations prevent p53-dependent transcription and consequently p53-mediated tumor suppression. The exceptionally high frequency of p53 mutations in human tumors of diverse types makes p53 unique among genes involved in tumor development, rendering mutated p53 (Mut-p53) an attractive target for novel cancer therapies.

Structural studies have revealed that the tumor-derived missense mutations in the DBD of p53 produce a common effect: destabilization of DBD folding at physiological temperature (Joerger, A. C., M. D. Allen, and A. R. Fersht, Crystal structure of a superstable mutant of human p53 core domain. Insights into the mechanism of rescuing oncogenic mutations. J Biol Chem, 2004 279(2): p. 1291-6). This destabilization may be reversible, since some mutants can revert to wild-type conformation and bind DNA at reduced temperatures. Thus, most mutations of p53 destabilize p53 protein folding, causing partial denaturation at physiological temperature.

Mutant p53 proteins accumulate at high levels in tumor cells, mainly due to their inability to upregulate the expression of p53's own destructor Mdm2. Moreover, many p53 activating stress signals (like hypoxia, genomic instability and oncogene expression) are constitutively induced in cancer cells. Therefore, reactivation of Mut-p53 is expected to exert major anti-tumor effects. Furthermore, it has been shown in a mouse model that restoration of p53 functions is well tolerated in normal tissues and produces no visible toxic effects (Ventura, A., et al., Restoration of p53 function leads to tumour regression in vivo. Nature, 2007. 445(7128): p. 661-5).

Structural studies show that the extent of misfolding differs among mutants; however, there is no defined alternative fold but rather a partial denaturation. This suggests that a “small molecule” approach to reverse the effect of p53 mutation on folding could be applicable to a wide range of mutant forms. Another important prediction from structural studies is that a ligand that binds to the properly folded fraction of the protein is expected to shift the equilibrium towards the native fold according to the law of mass action.

Several correctional approaches were attempted in the p53 conformation field. Proof of principle for conformation stabilizing peptides was provided by Friedler and colleagues (Friedler, A., et al., A peptide that binds and stabilizes p53 core domain: chaperone strategy for rescue of oncogenic mutants. Proc. Natl. Acad. Sci. USA, 2002. 99(2): p. 937-42). A nine-residue peptide, CDB3, was designed based on the crystal structure of the complex between the p53 DBD and ASPP (Samuels-Lev, Y., et al., ASPP proteins specifically stimulate the apoptotic function of p53. Mol. Cell, 2001. 8(4): p. 781-94). This peptide was shown to bind Mut-p53 and act as a chaperone, shifting equilibrium towards the WT conformation, as indicated by increased reactivity to PAb1620. However, the biological effects of CDB3 (Issaeva, N., et al., Rescue of mutants of the tumor suppressor p53 in cancer cells by a designed peptide. Proc. Natl. Acad. Sci. USA, 2003. 100(23): p. 13303-7) are only partial since the conformation of the Mut-p53/CDB3 complex is in an intermediate state between WT and mutant.

Small molecule compounds targeting Mut-p53 have been identified using either protein-based or cell-based assays (Peng, Y., et al., Rescue of mutant p53 transcription function by ellipticine. Oncogene, 2003. 22(29): p. 4478-87). CP-31398 was identified by screening for molecules that protect the isolated p53 DBD from thermal denaturation, as assessed by maintenance of PAb1620 reactivity upon protein heating (Foster, B. A., et al., Pharmacological rescue of mutant p53 conformation and function. Science, 1999. 286(5449): p. 2507-10). The mechanism of action of CP-31398 remains unclear. NMR studies failed to detect any binding of CP-31398 to the p53 DBD (Rippin, T. M., et al., Characterization of the p53-rescue drug CP-31398 in vitro and in living cells. Oncogene, 2002. 21(14): p. 2119-29). CP-31398 affects gene expression and induces cell death both in a p53-dependent and independent manner. Thus, it appears that CP-3138 has other cellular targets than p53 that may account for its cellular toxicity.

Two other small molecules that rescue p53 function in living cancer cells, PRIMA-1 and MIRA-1, were discovered by using cell-based screening assays. PRIMA-1 and MIRA-1 have similar activity profiles (Bykov, V. J., et al., Reactivation of mutant p53 and induction of apoptosis in human tumor cells by maleimide analogs. J Biol Chem, 2005. 280(34): p. 30384-91), but are structurally unrelated. PRIMA-1 is a pro-drug, which is converted into an active compound that binds to mutant p53 but also to other molecules (Cell Death Dis. 2015 Jun. 18; 6:e1794. doi: 10.1038/cddis.2015.143.), and some of its effects appear to be independent of mutant p53 status (BMC Cancer. 2015 Oct. 13; 15:684. doi: 10.1186/s12885-015-1667-1.). Inventors of some embodiments of the invention have previously described the use of phage display to select mutp53-reactivating peptides (WO2015/019318). Phage peptide display libraries have a much higher complexity than chemical libraries. The selection process was based on binding of peptides to an immobilized target, elution and amplification and finally identification by sequencing, enabling screening of high numbers of molecules in a short time. Different selection strategies were combined to select leads from different peptide libraries and deep sequencing of selected pools. Lead peptides were shown to endow mutp53 with WTp53-like activities in vitro and in live cells, and cause regression of mutp53-bearing tumors in several xenograft models.

Bromodomain and extraterminal motif (BET) protein inhibition is a promising cancer treatment strategy. The BET inhibitor BAY1238097 has been shown to have anti-tumor activity in preclinical lymphoma models (Bernasconi et al. British Journal of Haematology, 2017, 178, 936-948). Gene expression profiling showed that BAY1238097 targeted the NFKB/TLR/JAK/STAT signaling pathways, MYC and E2F1-regulated genes, cell cycle regulation and chromatin structure.

There is an unmet need for additional therapeutic agents for treating hematologic cancers, such as lymphoma and multiple myeloma, either as stand-alone treatments or combined with other therapies.

### SUMMARY OF THE INVENTION

According to an aspect of some embodiments of the present invention there is provided an isolated peptide comprising an amino acid sequence arranged in a space and configuration that allow interaction of the peptide with the DNA Binding Domain (DBD) of p53 through at least one residue of the DBD by which pCAP 250 (SEQ ID NO: 1) binds the DBD, wherein the peptide at least partially reactivates a mutant p53 protein, with the proviso that the peptide is not SEQ ID NO: 59-382.

According to some embodiments of the invention, the interaction is via Helix-2 and L1 of the DBD.

According to some embodiments of the invention, the interaction affects the structural stability of Helix-2 and/or L1 of the DBD, as assayed by NMR. According to some embodiments of the invention, the at least one residue is selected from the group consisting of H115, G117 of L1 and Y126 and V274 and G279 and R280 of the p53.

According to some embodiments of the invention, the interaction is by at least one amino acid of the amino acid sequence.

According to some embodiments of the invention, the interaction is by at least two amino acids of the amino acid sequence.

According to some embodiments of the invention, the interaction is by at least three amino acids of the amino acid sequence.

According to some embodiments of the invention, the interaction is by at least four amino acids of the amino acid sequence.

According to some embodiments of the invention, the peptide comprises an amino acid sequence of:

X₁-X₂-X₃-X₄-X₅-X₆ (SEQ ID NO: 53)

wherein,
X₁ and X₅ are a positively charged amino acid;
X₂ is selected from the group consisting of Ser, Thr, Asn, Gln, Pro, Ala and Gly;
X₃ is any amino acid;
X₄ and X₆ are selected from the group consisting of an alpha methyl amino acid and a beta breaker amino acid.

According to some embodiments of the invention, the peptide comprises an amino acid sequence of:

X₁-X₂-X₃-X₄-X₅-X₆ (SEQ ID NO: 54)

wherein,
X₁ and X₅ are selected from the group consisting of His, Arg and Lys;
X₂ is selected from the group consisting of Ser, Thr, Asn, Gln, Pro, Ala and Gly;
X₃, X₄, X₆ is any amino acid.

According to some embodiments of the invention, the positively charged amino acid is selected from the group consisting of His, Diaminobutyric acid (Dab), Arg and Lys.

According to some embodiments of the invention, the X₃ is a D-amino acid.

According to some embodiments of the invention, the X₃ is a phosphorylated amino acid.

According to some embodiments of the invention, X₃ is a non-phosphorylatable amino acid.

According to some embodiments of the invention, the X₃ is a non-hydrogen bonding amino acid.

According to some embodiments of the invention, the X₃ is selected from the group consisting of polar uncharged amino acid and a hydrophobic amino acid.

According to some embodiments of the invention, the X₂ is Ser.

According to some embodiments of the invention, the X₄ is alpha methyl amino acid and X₆ is alanine.

According to some embodiments of the invention, the isolated peptide has the amino acid sequence HSAPHP (SEQ ID NO: 49) or HSEPHP (SEQ ID NO: 50).

According to some embodiments of the invention, the isolated peptide comprises at least one additional amino acid (X₇) attached to the C-terminus of the amino acid sequence.

According to some embodiments of the invention, the at least one additional amino acid is a negatively charged amino acid.

According to some embodiments of the invention, the at least one additional amino acid is selected from the group consisting of Asp, Glu, Gly, Ala and Ser.

According to some embodiments of the invention, the at least one additional amino acid comprises two additional amino acids (X₇-X₈) and wherein the X₈ is selected from the group consisting of His, Dab, Asp and Glu.

According to some embodiments of the invention, the isolated peptide comprises at least one additional amino acid attached to the N-terminus of the amino acid sequence.

According to some embodiments of the invention, the isolated peptide comprises at least two additional amino acids attached to the N-terminus of the amino acid sequence.

According to some embodiments of the invention, the at least one additional amino acid attached to the N-terminus of the amino acid sequence is Arg.

According to some embodiments of the invention, the isolated peptide further comprises a cell penetrating moiety.

According to some embodiments of the invention, the cell penetrating moiety is attached to an N-terminus of the peptide.

According to some embodiments of the invention, the cell penetrating moiety is selected from the group consisting of a fatty acid moiety, a proteinaceous moiety and a combination of same.

According to some embodiments of the invention, the fatty acid moiety comprises a myristoyl fatty acid and the proteinaceous moiety comprises at least one positively charged amino acid.

According to some embodiments of the invention, the isolated peptide is no longer than 20 amino acids in length.

According to some embodiments of the invention, the peptide at least partially changes the conformation of the mutant p53 protein to a conformation of a wild-type (WT) p53 protein.

According to some embodiments of the invention, the peptide at least partially changes the conformation of the mutant p53 protein such that the mutant p53 protein is recognized by a monoclonal antibody directed against a WT p53 protein.

According to some embodiments of the invention, the mutant p53 protein is not recognized by a monoclonal antibody directed against a WT p53 protein.

According to some embodiments of the invention, the mutant p53 protein, upon binding to the peptide, is recognized by a monoclonal antibody directed against a WT p53 protein.

According to some embodiments of the invention, the monoclonal antibody is Ab1620.

According to some embodiments of the invention, the peptide at least partially restores the activity of the mutant p53 protein to the activity of a WT p53 protein.

According to some embodiments of the invention, the activity is reducing viability of cells expressing the mutant p53 protein.

According to some embodiments of the invention, the activity is promoting apoptosis of cells expressing the mutant p53 protein.

According to some embodiments of the invention, the activity is binding to a p53 consensus DNA binding element in cells expressing the mutant p53 protein.

According to some embodiments of the invention, the consensus DNA binding element comprises the nucleic acid sequences set forth in SEQ ID NO: 55 and 56).

According to some embodiments of the invention, the binding results in at least partial activation of an endogenous p53 target gene.

According to some embodiments of the invention, the endogenous target gene is selected from the group consisting of p21, MDM2 and PUMA.

According to some embodiments of the invention, the mutant p53 protein is of a different conformation than a WT p53 protein.

According to some embodiments of the invention, the isolated peptide is as set forth in SEQ ID NO: 429 or 448.

According to some embodiments of the invention, the isolated peptide is as set forth in SEQ ID NO: 429, 448, 446, 449 or 462.

According to some embodiments of the invention, the isolated peptide is selected from the group consisting of SEQ ID NO: 8 and 412-464.

According to some embodiments of the invention, the isolated peptide is not any of the peptides set forth in SEQ ID NOs: 59-382.

According to an aspect of some embodiments of the present invention there is provided a method of treating a disease, disorder or condition associated with a mutant p53 protein, comprising administering to a subject in need thereof a therapeutically effective amount of the isolated peptide of as described herein, thereby treating the disease, disorder or condition.

According to some embodiments of the invention, the method further comprises administering to the subject a therapeutically effective amount of a platinum-based chemotherapy.

According to some embodiments of the invention, the disease is cancer.

According to some embodiments, the cancer is hematologic cancer. Thus, a method of treating a hematologic cancer is provided, comprising administering to a subject in need thereof an isolated peptide comprising an amino acid sequence arranged in a space and configuration that allow interaction of the peptide with the DNA Binding Domain (DBD) of p53 through at least one residue of the DBD by which pCAP 250 (SEQ ID NO: 1) binds the DBD, wherein the peptide at least partially reactivates a mutant p53 protein, with the proviso that the peptide is not SEQ ID NO: 59-382.

According to some embodiments, the peptide comprising an amino acid sequence selected from the group consisting of SEQ ID Nos: 1, 426, 427, 429, 430, 431, 443, 446, 448, 449, 453, 457, 458 and 462. Each possibility represents a separate embodiment of the invention. According to certain exemplary embodiments, the peptide is pCAP-553 (SEQ ID NO: 429). According to additional exemplary embodiments, the peptide is pCAP250 (SEQ ID NO: 1).

According to some embodiments, the cancer is lymphoma. According to certain embodiments, the lymphoma is non-Hodgkin lymphoma or Hodgkin lymphoma. According to additional embodiments, the lymphoma is small lymphocytic lymphoma (SLL).

According to additional embodiments, the cancer is multiple myeloma.

According to some embodiments, the method further comprises administering to the subject a therapeutically effective amount of an inhibitor of Bromodomain (BRD) and Extra-Terminal domain (BET) family. According to certain embodiments, the BET inhibitor is Bay1238097.

According to some embodiments, the isolated peptide described herein and the BET inhibitor are administered substantially simultaneously, concurrently, alternately, sequentially or successively. According to certain embodiments, the isolated peptide described herein and the BET inhibitor are administered according to overlapping schedules.

According to an aspect of some embodiments of the present invention there is provided a method of treating a disease, disorder or condition associated with a mutant p53 protein, comprising administering to a subject in need thereof a therapeutically effective amount of a platin-based chemotherapy and an isolated peptide comprising an amino acid sequence having a space and configuration that allow binding of the peptide to the DNA Binding Domain (DBD) of p53 in the same mode as pCAP 250 (SEQ ID NO: 1) binds the DBD, wherein the peptide at least partially reactivates a mutant p53 protein, thereby treating the disease, disorder or condition.

According to an aspect of some embodiments of the present invention there is provided a method of treating a disease, disorder or condition associated with a mutant p53 protein, comprising administering to a subject in need thereof a therapeutically effective amount of an isolated peptide comprising an amino acid sequence having a space and configuration that allow binding of the peptide to the DNA Binding Domain (DBD) of p53 in the same mode as pCAP 250 (SEQ ID NO: 1) binds the DBD, wherein the peptide at least partially reactivates a mutant p53 protein and wherein the therapeutically effective amount is 0.01-0.3 mg/kg per day, thereby treating the disease, disorder or condition.

According to some embodiments of the invention, the peptide is the peptide as described herein.

According to some embodiments of the invention, the peptide is pCAP 250 (SEQ ID NO: 1).

According to some embodiments of the invention, the administering comprises subcutaneous administering.

According to some embodiments of the invention, the administering comprises continuous infusion.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a dose response of pCAP-250 (SEQ ID NO: 1) alone or in combination with Cisplatin in viability assay of ES2 ovarian cancer cells. Cells were cultured in 96 wells plates with 3000 cells/well. Serial dilutions of pCAP-250 were added either alone or together with 1 μg/ml of cisplatin and the plates incubated for additional 48 h at 37° C. Then medium was removed and cell viability was determined by staining the cells with crystal violet (0.05%) in methanol/PBS (1:5, v/v), for 10 min, followed by 3 washes with PBS. 10% acetic acid was added to each well for 10 min. OD was determined at 595 nm. The viability of ES2 cells treated with 1 μg/ml was 39%. The IC50 for pCAP-250 was estimated at 3.2 μM and in combination with cisplatin the IC50 for pCAP-250 was estimated at 1.9 μM indicating a synergistic effect between the two compounds.

FIG. 2 is a bar graph showing the effect of pCAP-250 (SEQ ID NO: 1) and different derivatives (SEQ ID NOs: 2-19) in viability assay of ES2 ovarian cancer cells and on binding to p53 DBD as determined by MST. Cells, ES2 Con expressing endogenous mp53^{S241F}, and ES2 KO cells in which p53 was stably knocked out using CRISPR/Cas9 (ES2 p53KO), to control for specificity for mutp53 were cultured in 96 wells plates with 3000 cells/well. Indicated peptides were added at a concentration of 8 μg/ml and the plates incubated for additional 48 h at 37° C. Then medium was removed and cell viability was determined by staining the cells with crystal violet (0.05%) in methanol/PBS (1:5, v/v), for 10 min, followed by 3 washes with PBS. 10% acetic acid was added to each well for 10 min. OD was determined at 595 nm. The difference in the effect of a particular peptide for ES2 Con compared to ES KO indicates specificity of peptide to mutp53 expression. Several peptide derivatives in which amino acids that were substituted to Alanine (Serine and Histidine for example) showed a decreased effect on ES2 Con cells indicating the importance of these amino acids for peptide efficacy.

FIGS. 3A-3K are graphs of microscale thermophoresis (MST) analysis for the binding of fluorescently labeled WTp53DBD (FIG. 3A) or full length p53 (FIG. 3B) and the indicated peptides (SEQ ID NOs: 1, 4, 9). The experiment was performed according to the manufacturer's instructions; 10 serial dilutions of each indicated to peptide; (FIG. 3A—pCAP-250) (FIG. 3A, FIG. 3F, FIG. 3H, FIG. 3I, FIG. 3K pCAP402, pCAP 404, pCAP409 and pCAP 364) were prepared, labeled protein was added to each peptide sample and loaded to capillaries. The samples were analyzed for movement of fluorescent wtp53DBD in temperature gradient with different concentrations of peptides. MST analysis results are presented as a curve obtained from manufacturer data analysis software.

FIGS. 4A-4D show the pharmacokinetics of various modes of administration. FIG. 4A—Plasma concentration vs. time profiles of pCAP-250 after administration of 1 mg/kg iv (mean±SD, n=3). FIG. 4B—Plasma concentration vs. time profiles of pCAP-250 after continuous subcutaneous administration for 7 days (mean±SD, n=3).

FIG. 4C—Plasma concentration vs. time profiles of pCAP-250 after administration of 1 mg/kg iv (mean±SD, n=3). FIG. 4D—Plasma concentration vs. time profiles of PCAP-250 after subcutaneous administration of 1 mg/kg (mean±SD, n=3).

FIGS. 5A-5D In-vivo effect of pCAP-250 peptide in a mouse xenograft model.

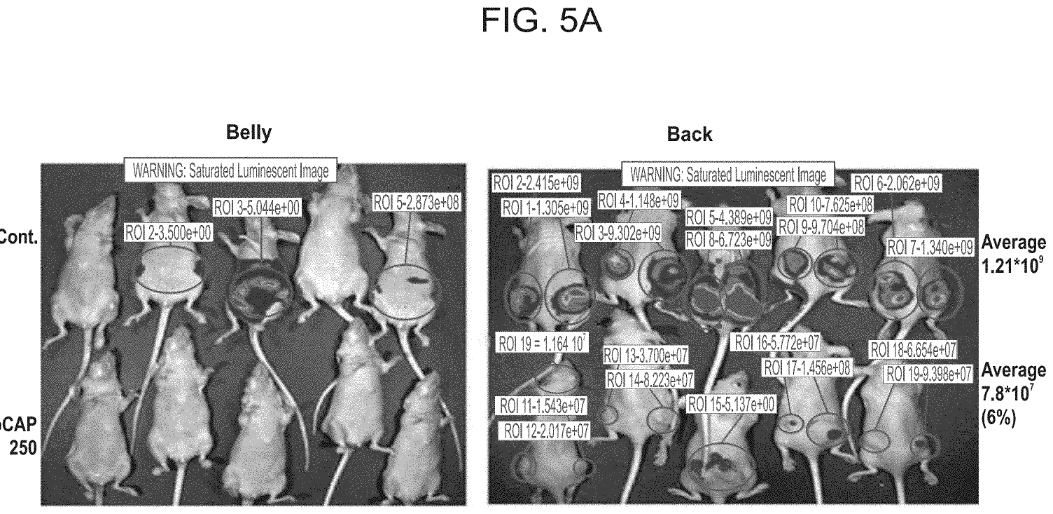

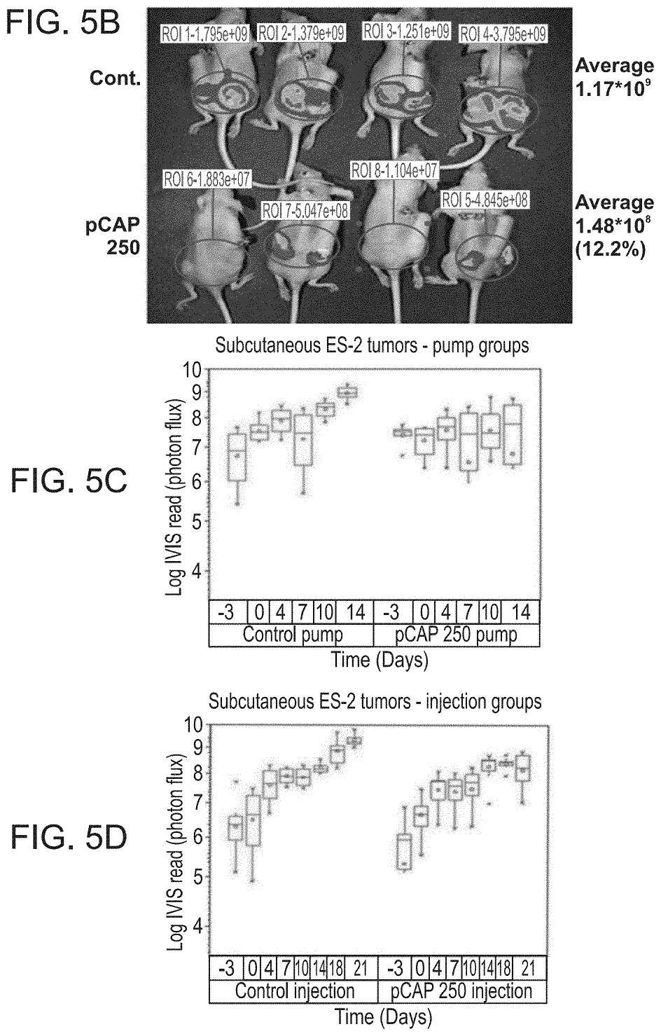

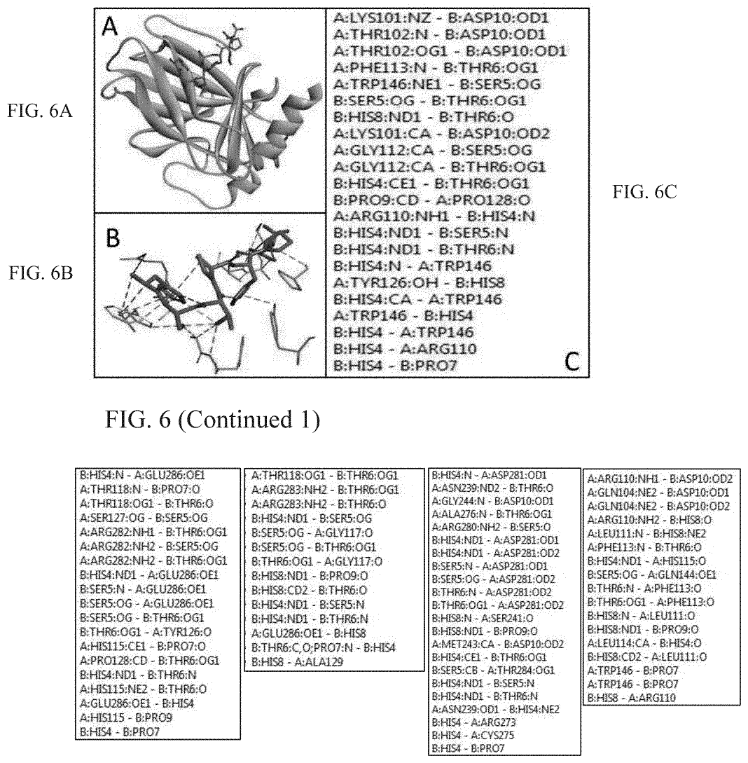

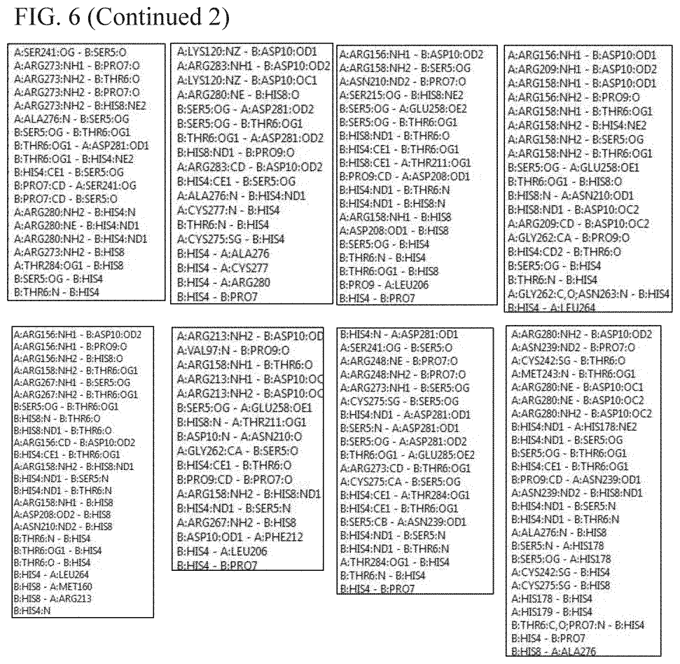

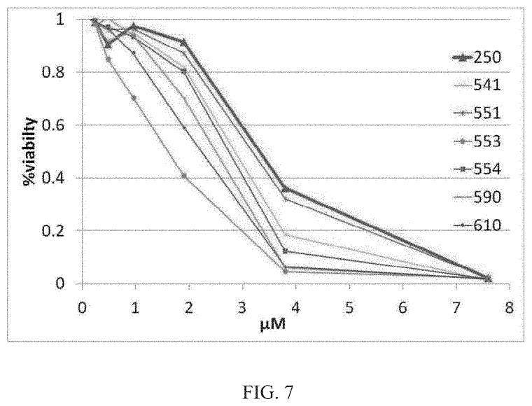

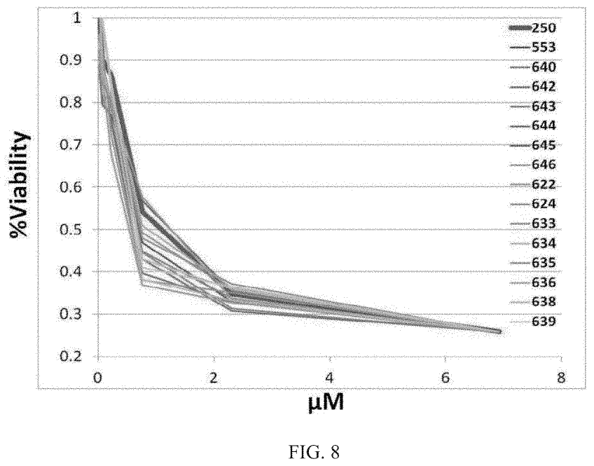

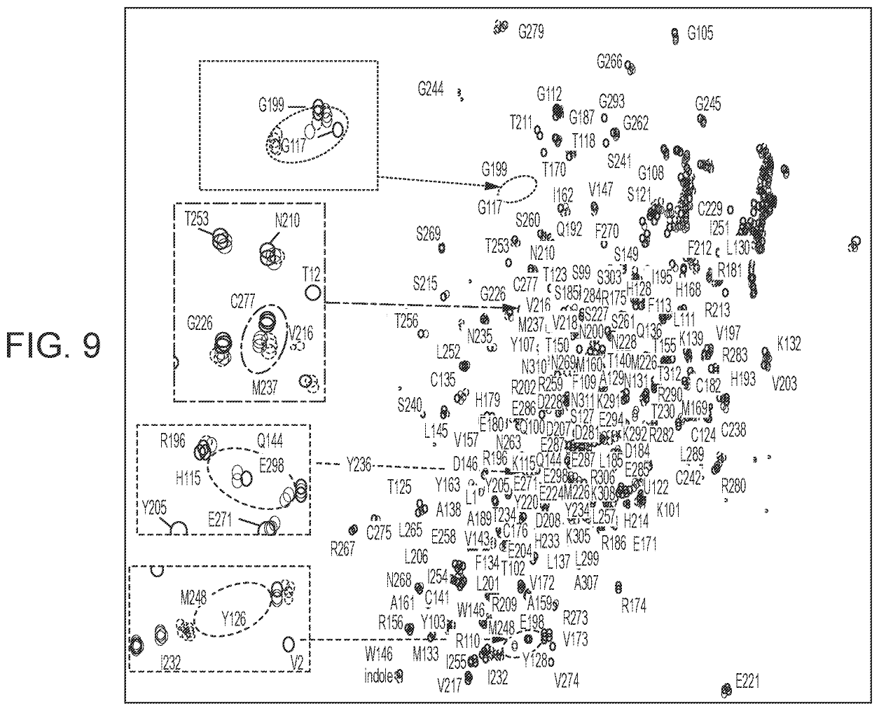

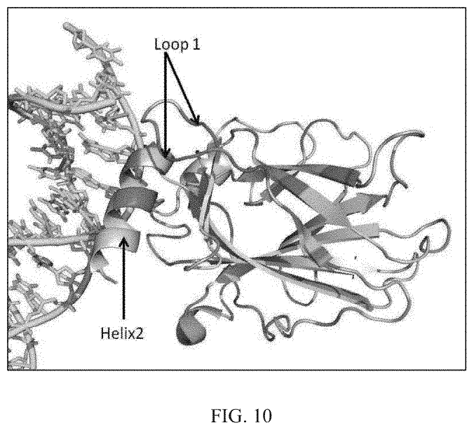

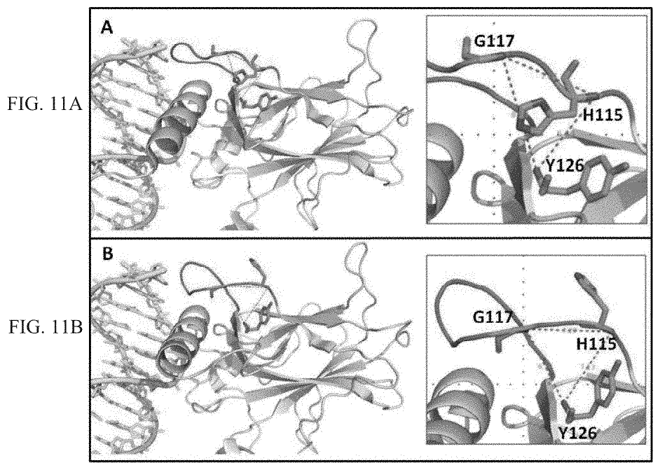

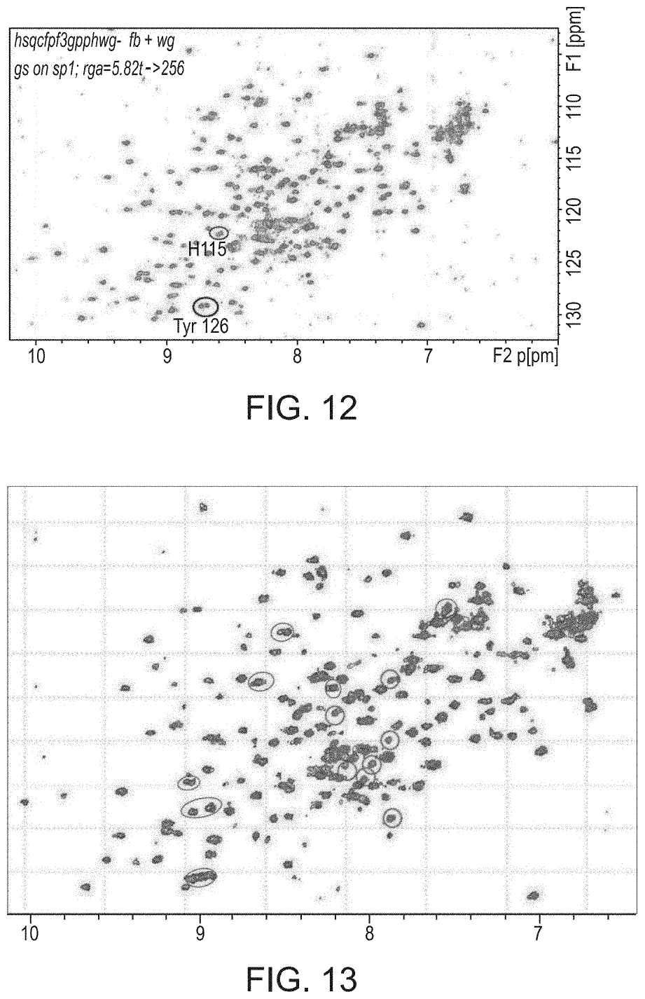

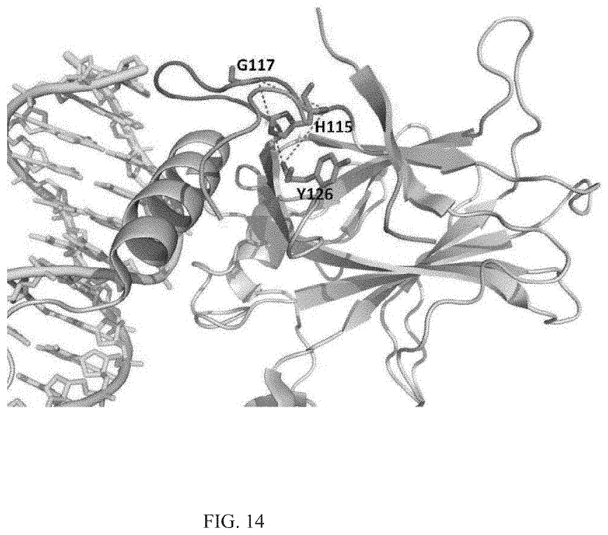

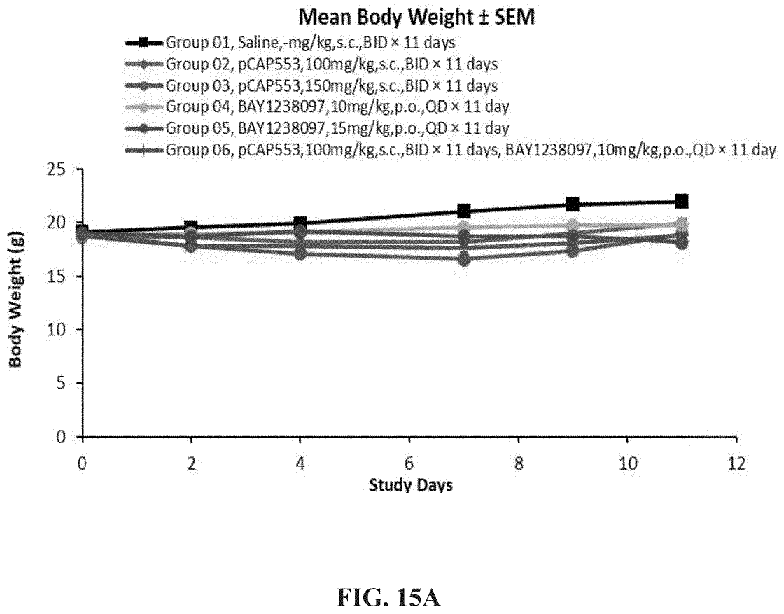

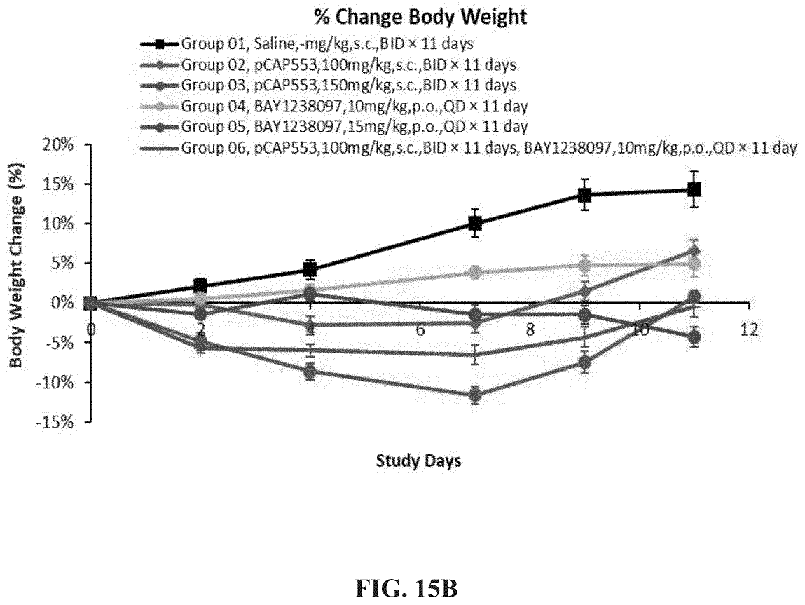

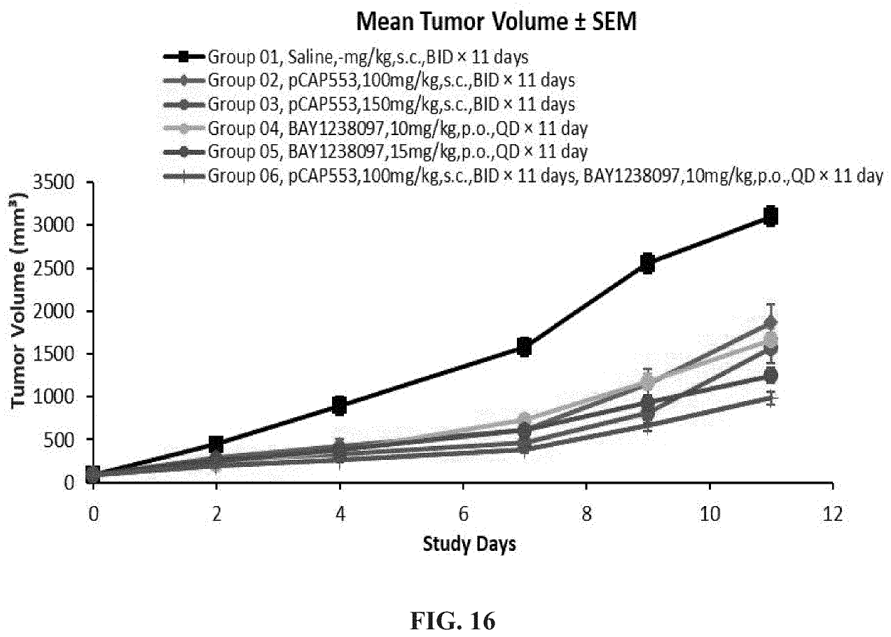

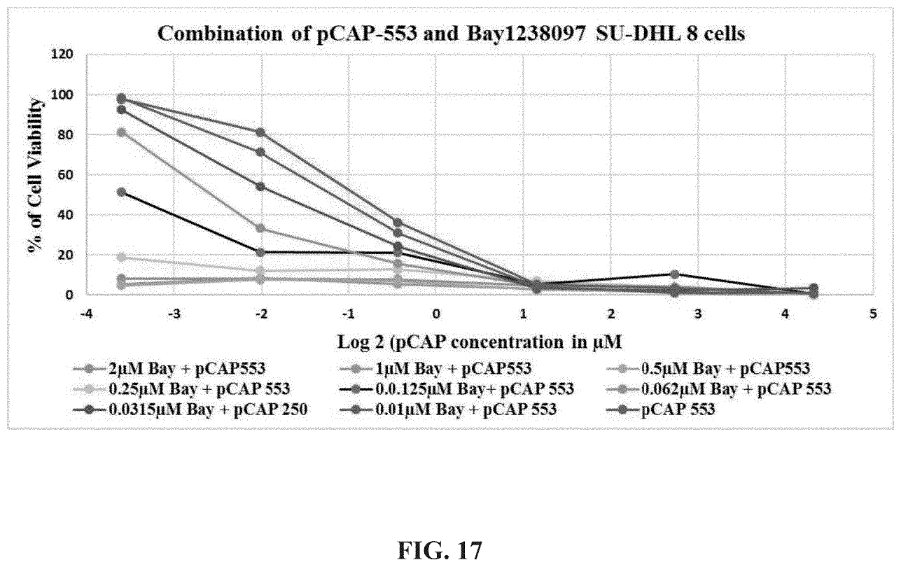

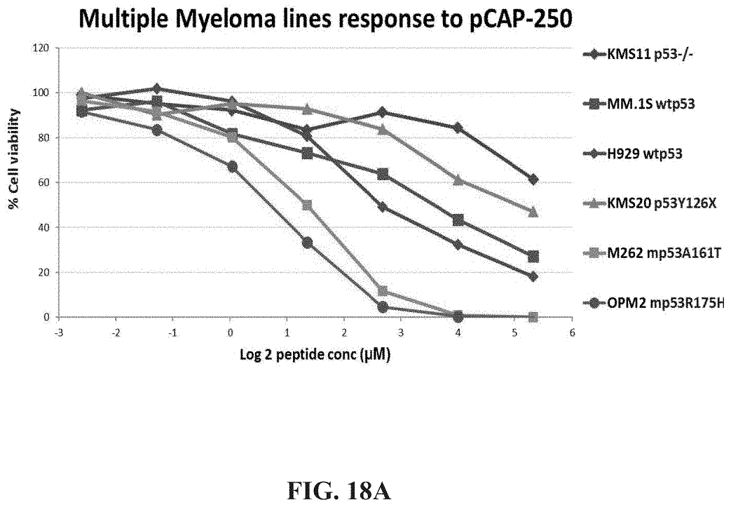

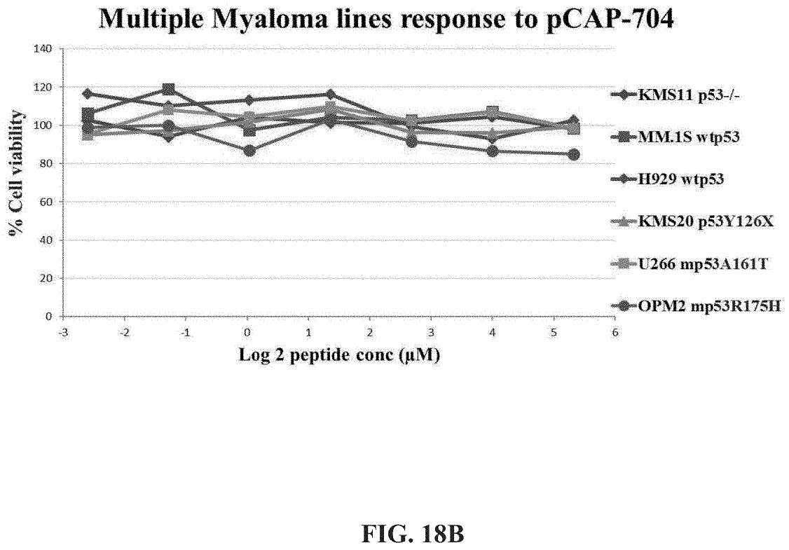

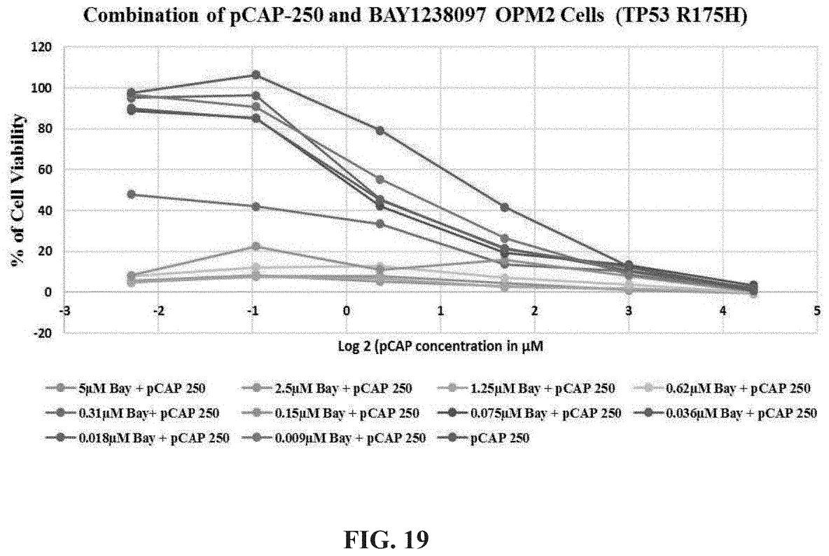

2*10⁵ ES2 cells expressing luciferase were injected into the hips of nude mice. Bioluminescence was measured. 12 days after injection, mice were randomly divided to 4 groups and either injected intratumorally, three times a week, with a mixture of 2 control peptides (pCAPs 76 and 12; 5 μg of each peptide) or pCAP-250 (10 μg). Alternately, mice were transplanted with Alzet minipumps containing 0.8 mg in PBS control peptides or 0.8 mg in PBS of pCAP-250. FIG. 5A, Live imaging of control group mice and intratumoral pCAP-250 treated mice, at termination of experiment (day 21). FIG. 5B—Live imaging of control group mice and Alzet minipumps pCAP-250 treated mice, at termination of experiment (day 14). FIG. 5C—control mice and effective pCAP-250 group: box-plot showing the luciferase readings in tumors as a function of time; average (horizontal line), standard deviation (box), highest and lowest reads are shown, before (until day 0) and after initiation of treatment. The background threshold detection level of the IVIS system was about 5×10⁶ photons. FIG. 5D-Control mice and effective pCAP-250 group: box-plot showing the luciferase readings in tumors as a function of time; average (horizontal line), standard deviation (box), highest and lowest reads are shown, before (until day 0) and after initiation of treatment. The background threshold detection level of the IVIS system was about 5×10⁶ photons.

FIGS. 6A-6C show optional predicted peptide binding position for the HSTPHPD peptide sequence on the surface of the P53 DNA binding domain (DBD). The DBD is shown in carton cyan representation and the predicted peptide is shown as magenta sticks. FIG. 6A. An overview of the DBD peptide complex. FIG. 6B. A closer examination of the DBD-peptide binding interface. FIG. 6C. A detailed atomic list of the non-bonded interaction between the DBD (chain B) and the predicted peptide binding position (chain A).

FIG. 7 shows dose response effects of p53-reactivating peptides in triplicates. SW480 cell line comprising p53 mutant p53R273H. Cells were cultured in 96 wells plates with 3000 cells/well. Serial dilutions of different peptides were added and the plates incubated for additional 72 h at 37° C. Then the medium was removed and cell viability was determined by staining the cells with crystal violet (0.05%) in methanol/PBS (1:5, v/v), for 10 min, followed by 3 washes with PBS. 10% acetic acid was added to each well for 10 min. OD was determined at 595 nm. Results are normalized to non-treated cells 100% viability.

FIG. 8 shows dose response effects of p53-reactivating peptides in triplicates. ES2 cell line comprising p53 mutant S241F. Cells were cultured in 96 wells plates with 3000 cells/well. Serial dilutions of different peptides were added and the plates incubated for additional 48 h at 37° C. Then the medium was removed and cell viability was determined by staining the cells with crystal violet (0.05%) in methanol/PBS (1:5, v/v), for 10 min, followed by 3 washes with PBS. 10% acetic acid was added to each well for 10 min. OD was determined at 595 nm. Results are normalized to non-treated cells 100% viability.

FIG. 9 shows 1H-15N HSQC spectra of wild-type p53 core domain (DBD) acquired at 293 K, DBD (94-312 of SEQ ID NO: 44) spectra and residue assignment as was produced by Wong et al is shown in black [Wong, K. B., et al., Hot-spot mutants of p53 core domain evince characteristic local structural changes. Proc Natl Acad Sci USA, 1999. 96(15): p. 8438-42]. NMR spectra produced for the free DBD (94-296) and for the DBD-pCAP 250 complex are shown in blue and red, respectively. Examples of moderate (C277 and R280) and strong peak changes (G117) are emphasized in magenta and brown respectively. The peak region of H115 and Y126 are emphasized In yellow.

FIG. 10 shows mapping of the DBD structure for 1H-15N HSQC spectra changes as a result of the binding of pCAP 250 (SEQ ID NO: 1) to the DBD. The DBD structure is shown in cartoon representation and the DNA is colored yellow. Unassigned residues from the analysis of Wong et al. (supra) are colored green and residues involving peak changes upon the addition of pCAP 250 are colored magenta.

FIGS. 11A-11B show the structural reorganization of H115, G117 and Y126. The DBD structure is shown in cartoon representation and the DNA is colored yellow. H115, G117 and Y126 are shown as green sticks and the L1 loop is colored magenta. FIGS. 11A and 11B present the top and the second top best energy DBD conformations solved by NMR (pdb code 2FEJ), respectively.

FIG. 12 show 1H-15N HSQC spectra of wild-type p53 DBD-peptide complexes acquired at 293 K. NMR spectra produced for the DBD-pCAP 250 and for the DBD-pCAP 615 (SEQ ID NO: 465) protein peptide complexes are shown in red and green, respectively. The peaks of H115 and Y126 are emphasised as circles.

FIG. 13 show 1H-15N HSQC spectra of wild-type p53 DBD and DBD-pCAP 553 (SEQ ID NO: 429)-complex acquired at 293 K. NMR spectra produced for the free DBD and for the DBD-pCAP 553 protein peptide complex are shown in blue and red respectively. Strong unassigned peaks that specifically emerged up on the edition of the pCAP 553 peptide are emphasized as green ellipsoids. Few examples of peaks which become more condensed and circular in the DBD-pCAP 553 complex are emphasized in brown ellipsoids.

FIG. 14 shows top two predicted peptide binding models for the DBD-pCAP 250 complex. The DBD structure is shown in cartoon representation and the DNA is colored yellow. H115, G117 and Y126 are shown as green sticks and the L1 loop is colored magenta. The top two predicted peptide binding models for the DBD-pCAP 250 complex are colored in cyan.

FIGS. 15A-15B show the effect of pCAP-553 (SEQ ID NO. 429) on body weight, mean (FIG. 15A) and % change (FIG. 15B) in lymphoma xenograft mouse model. The inhibitor of the Bromodomain (BRD) and Extra-Terminal domain (BET) family, Bay1238097, was used as positive control. BID—Twice a day, QD—once a to day, p.o.—Orally, s.c.—subcutaneously.

FIG. 16—shows the effect of pCAP-553 on tumor volume in lymphoma xenograft mouse model.

FIG. 17 shows the effect of different concentrations of pCAP-553 and Bay1238097 on cell viability of SU-DHL 8 cells.

FIGS. 18A-18B. show the correlation between p53 variants in human multiple myeloma cell lines and response to peptide pCAP-250 (SEQ ID NO: 1). Viability experiment performed with 6 multiple myeloma cell lines, differing in p53 protein variant. Serial dilutions of peptides were performed, and cells were then added to each well and incubated for 72 h. Cell viability was determined by CellTiter-Glo (CTG) (FIG. 18A). A scrambled sequence pCAP-704 was used as control (FIG. 18B).

FIG. 19 shows the effect of a combination of the peptide pCAP-250 (SEQ ID NO: 1) with Bay1238097 on cell viability of OMP1 cells.

### DESCRIPTION OF SPECIFIC EMBODIMENTS OF THE INVENTION

The present invention, in some embodiments thereof, relates to peptides and use of same in the treatment of diseases, disorders or conditions associated with a mutant p53.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Inventors of some embodiments of the invention have previously described the use of phage display to select mutp53-reactivating peptides (WO2015/019318, which is hereby incorporated by reference in its entirety). Lead peptides including pCAP 250 (SEQ ID NO: 1) were shown to endow mutp53 with WTp53-like activities in vitro and in live cells, and cause regression of mutp53-bearing tumors in several xenograft models.

Whilst reducing the present invention to practice, the present inventors have uncovered that pCAP 250 binds the DNA Binding Domain (DBD) of p53.

Structural/functional analysis using alanine scanning revealed a consensus for the binding of pCAP 250 to the DBD.

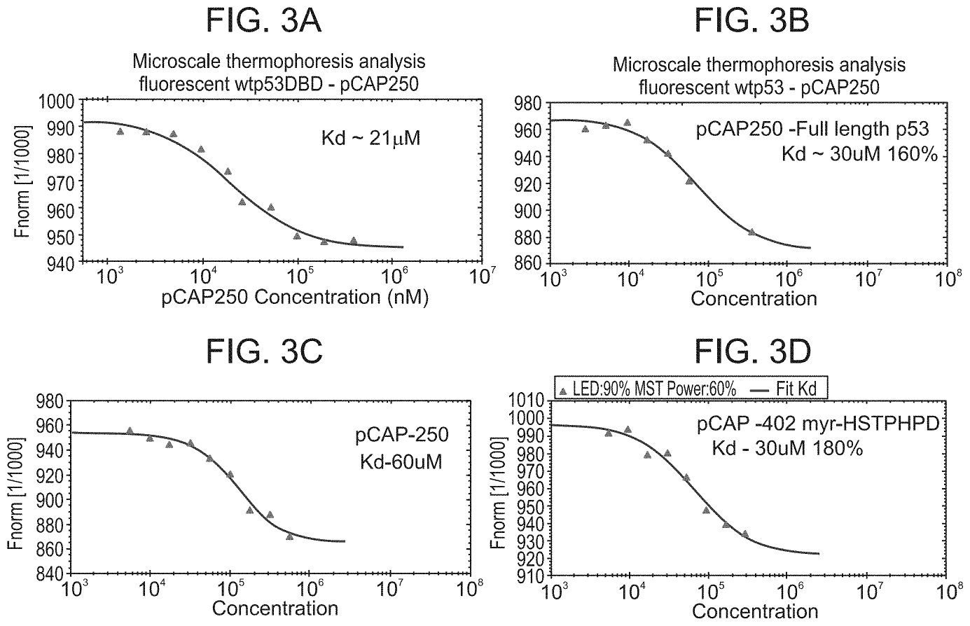

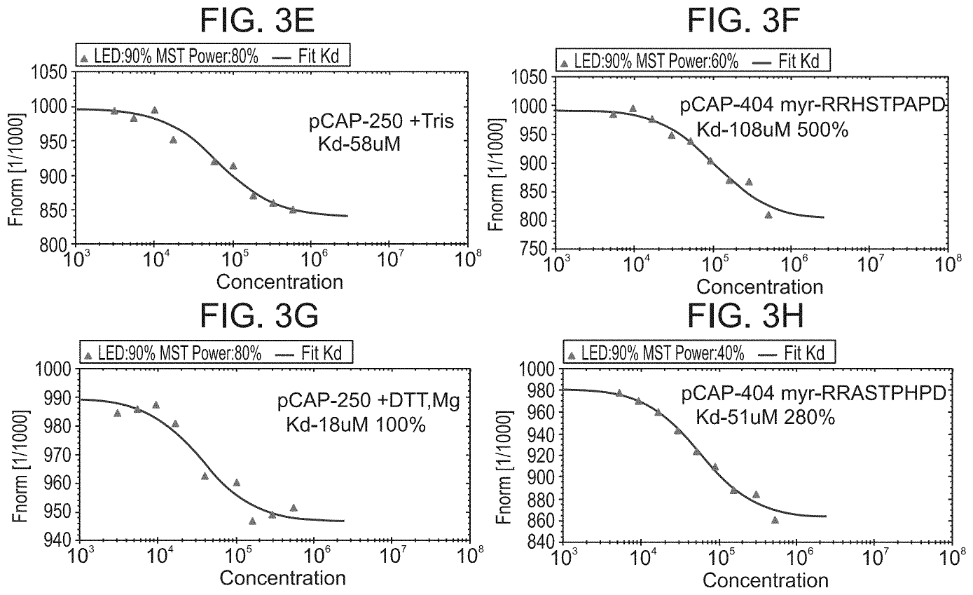

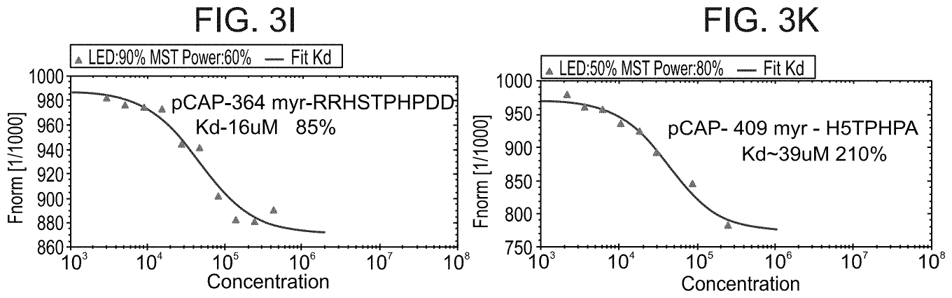

NMR experimental results provide further evidence for the explicit binding of pCAP 250 and its peptide variants to the WT DBD of the p53 protein. These results support the findings regarding the binding of pCAP 250 to the DBD using the microscale thermophoresis (MST) analysis (FIGS. 3A-K). The NMR results further indicate that the binding of pCAP 250 and its peptide variants induces structural changes in the DBD, which directly influence the integrity and stability of the DBD-DNA binding interface region, namely the Helix-2 and the L1 loop structural motifs, which are essential for the ability of the DBD to bind the DNA. The binding of pCAP 250 and its peptide variants further affects additional residues at the surroundings of the helix 2 and the L1 loop structural motifs, creating a relatively large yet decisive affected patch on the DBD surface.

These findings allow the design of novel peptides that share the same interaction with the DBD of p53 and are able to at least partially reactivate a mutant p53 protein such peptides endowed with anti-cancer activity are shown in Example 5.

Thus, according to an aspect of the present invention there is provided an isolated peptide comprising an amino acid sequence arranged in a space and configuration that allow interaction of the peptide with the DNA Binding Domain (DBD) of p53 through the same at least one residue of the DBD by which pCAP 250 (SEQ ID NO: 1) binds the DBD, wherein said peptide at least partially reactivates a mutant p53 protein.

According to a specific embodiment, the peptide is not SEQ ID NO: 1-338, 368-382 of WO2015/019318 (i.e., SEQ ID NOS: 59-382 herein).

According to a specific embodiment, the peptide is not any of the peptides taught in WO2015/019318 as having the activity of re-activating mutant p53, which is hereby incorporated by reference in its entirety.

As used herein the term “isolated” refers to at least partially separated from the natural environment e.g., from the body or from a peptide library.

As used herein the term “p53” also known as “TP53” refers to the gene sequence encoding the protein product of EC 2.7.1.37, generally functioning as a transcription factor, regulating the cell cycle, hence functioning, in its wild-type form, as a tumor suppressor gene. According to a specific embodiment, the p53 is a human p53.

As used herein, the terms “wild type p53”, “wt p53” and “WT p53” may interchangeably be used and are directed to a wild type p53 protein, having the conformation of a wild type p53 protein and hence, activity of a wild type p53 protein.

In some embodiments, wild type p53 can be identified by a specific monoclonal antibody. In certain embodiments, the monoclonal antibody is Ab1620.

Structural data for the protein is available from PDBe RCSB.

The term “conformation” with respect to a protein is directed to the structural arrangement (folding) of a protein in space.

As used herein, the terms “mutant p53”, “Mut-p53”, “mutated p53”, and “p53 mutant” may interchangeably be used and are directed to a mutated p53 protein, incapable of efficiently functioning in a target cell. In some embodiments, a Mut-p53 cannot bind its target site. In some embodiments, a Mut-p53 is mutated at the DNA binding domain (DBD) region. In some embodiments, a Mut-p53 is misfolded in an inactive conformation. In some exemplary embodiments, the Mut-p53 is a temperature sensitive (ts) mut p53R249S (R249S p53), a hot spot full length mutant p53 Mut-p53 R175H (R175H p53), or any other Mut-p53 protein. In some embodiments, a Mut-p53 is identified by a specific monoclonal antibody, capable of recognizing a misfolded conformation of p53 (induced by the mutation of the p53). In some embodiments, a Mut-p53 is identified by a specific monoclonal antibody. In certain embodiments, the monoclonal antibody is Ab420.

In certain embodiments, the mutant p53 protein comprises a mutation selected from the group consisting of R175H, V143A, R249S, R273H, R280K, P309S, P151S, P151H, C176S, C176F, H179L, Q192R, R213Q, Y220C, Y220D, R245S, R282W, D281G, S241F, C242R, R248Q, R248W, D281G, R273C and V274F. Each possibility represents a separate embodiment of the invention.

As referred to herein, the terms “reactivating peptide”, “Mut-p53 reactivating peptide” or “the peptide” may interchangeably be used and are directed to a peptide capable of at least partially restoring activity to Mut-p53. The phrase “reactivating mutant p53 protein” as used herein refers to a peptide which upon its interaction with a mutant p53 protein, the mutant p53 protein increases at least one of its activities, wherein the activities are the activities of a wild type p53 protein. For example, upon its interaction with a peptide provided by the present invention, a mutant p53 protein may increase, directly or indirectly, the expression of pro-apoptotic proteins such as caspases in a cancer cell, in a similar way to what would a wild type p53 protein do in a similar situation or suppress tumors in vivo as can be assayed using a xenograft mouse model of the disease.

Without being bound by theory it is suggested that the reactivating peptide binds the mut p53 in the DBD and thermodynamically stabilizes the WTp53 protein folding and hence restore tumor suppression function.

In some embodiments, the reactivating peptide can reactivate a Mut-p53 by affecting the conformation of the Mut-p53, to assume a conformation which is more similar to or identical to a native, WT p53. In some embodiments, the reactivating peptide can reactivate a Mut-p53 to restore binding of the Mut-p53 to a WT p53 binding site in a target DNA. In some embodiments, the reactivating peptide can restore biochemical properties of the Mut-p53. In some embodiments, the reactivating peptide can induce the Mut-p53 protein to exhibit p53-selective inhibition of cancer cells. In some embodiments, the reactivating peptide can reactivate a Mut-p53 to have structural properties, biochemical properties, physiological properties and/or functional properties similar (i.e., ±, 10%, 20%, 30% difference between the Mut-p53 and WT p53) to or identical to a WT p53 protein such as determined in the binding/structural assays as described herein e.g., MST and NMR.

In some embodiments, the reactivating peptide is a peptide having 3-30 amino acids in length. In some embodiments, the reactivating peptide is a peptide having 7-30 amino acids in length. In some embodiments, the reactivating peptide is a peptide having 12-30 amino acids in length. In some embodiments, the reactivating peptide is a peptide having 3-25 amino acids in length. In some embodiments, the reactivating peptide is a peptide having 7-25 amino acids in length. In some embodiments, the reactivating peptide is a peptide having 12-25 amino acids in length. In some embodiments, the reactivating peptide is a peptide having 3-22 amino acids in length. In some embodiments, the reactivating peptide is a peptide having 7-22 amino acids in length. In some embodiments, the reactivating peptide is a peptide having 12-22 amino acids in length. In some embodiments, the reactivating peptide is a peptide having 7-9 amino acids in length. In some embodiments, the reactivating peptide is a peptide having 6-9 amino acids in length. In some embodiments, the reactivating peptide is a peptide having 7-10 amino acids in length. In some embodiments, the reactivating peptide is a peptide having 6-10 amino acids in length. In some embodiments, the reactivating peptide is a peptide being 9-10 amino acids in length. In some embodiments, the reactivating peptide is a peptide being 8-10 amino acids in length. In some embodiments, the reactivating peptide is a peptide being 6-9 amino acids in length. In some embodiments, the reactivating peptide is a peptide being 6-8 amino acids in length. In some embodiments, the reactivating peptide is a peptide being 6-7 amino acids in length. In some embodiments, the reactivating peptide is a peptide being 7-8 amino acids in length. In some embodiments, the reactivating peptide is a peptide being 7-9 amino acids in length. In some embodiments, the reactivating peptide is a peptide being 5-20 amino acids in length. In some embodiments, the reactivating peptide is a peptide being 6-15 amino acids in length. In some embodiments, the reactivating peptide is a peptide being 7 or 12 amino acids in length.

The term “capable of at least partially reactivating a mutant p53 protein” or “at least partially reactivate a mutant p53 protein” as interchangeably used herein refers to a peptide, wherein upon binding of the peptide to a mutant p53 protein, the mutant p53 protein gains or increases an activity similar to a corresponding activity of a wild type p53 protein.

As used herein “the DNA Binding Domain” or “DBD” of p53 refers to the domain of p53 which binds a p53 responsive element in a target protein (e.g., a consensus DNA binding element comprises or consists the amino-acid sequence set forth in SEQ ID NO: 44), typically attributed to residues 94-292, 91-292, 94-293, 94-296, 91-296, 91-293, 94-312 or 92-312 of human p53 (full length p53 GenBank: BAC16799.1, SEQ ID NO: 44). According to a specific embodiment, the DBD is of a mutated p53.

As mentioned, the peptide comprises an amino acid sequence arranged in a space and configuration that allow interaction of the peptide with the DBD of p53 through at least one residue of the DBD by which pCAP 250 (SEQ ID NO: 1) binds the DBD.

Thus, a reactivating peptide according to some embodiments of the invention is typically associated with the DBD domain of p53 such that the reactive group(s) of the peptide are positioned in a sufficient proximity to corresponding reactive group(s) (typically side chains of amino acid residues) in the DBD, so as to allow the presence of an effective concentration of the peptide in the DBD and, in addition, the reactive groups of the peptide are positioned in a proper orientation, to allow overlap and thus a strong chemical interaction and low dissociation. A reactivating peptide, according to some embodiments of the invention therefore typically includes structural elements that are known to be involved in the interactions, and may also have a restriction of its conformational flexibility, so as to avoid conformational changes that would affect or weaken its association with DBD of p53.

According to some embodiments, the interaction is via Helix-2 and L1 of said DBD.

Typically, helix-2 is positioned between amino acids 276-289 and L1 is positioned between amino acids 112-124.

According to some embodiments, the interaction affects the structural stability of Helix-2 and/or L1 of said DBD, as assayed by NMR.

According to some embodiments, the at least one residue in the DBD by which the interaction with the peptide is mediated is selected from the group consisting of H115, G117 of L1 of the p53 and Y126 and V274 and G279 and 8280 of the p53 (wt or mutant in which the difference in amino acids is typically of single amino acids that do not significantly affect amino acid numbering. However, the skilled artisan would know how to find the corresponding amino acid (in terms of composition and position in the mutant p53).

According to some embodiments the interaction of the peptide with the DBD is non-covalent, e.g., water-mediated hydrogen bonding interactions.

According to some embodiments the interaction is by at least one amino acid of the amino acid sequence.

According to some embodiments the interaction is by at least two amino acids of the amino acid sequence.

According to some embodiments the interaction is by at least three amino acids of the amino acid sequence.

According to some embodiments the interaction is by at least four amino acids of the amino acid sequence.

According to a specific embodiment, the interaction is to amino acid Trp146 and/or Gln144 of human p53. This interaction is probably via the Ser of the pCAP 250 or its likes in analogous structures as further described hereinbelow.

According to a specific embodiment, the interaction is to amino acid Tyr126, Asn128 and/or Asp268 of human p53.

According to another specific embodiment, the interaction is to amino acid Lys101 of human p53 via Asp10 of the pCAP 250 or its likes in analogous structures as further described hereinbelow.

According to another specific embodiment, the interaction is to amino acid Thr102 of human p53 via Asp10 of the pCAP 250 or its likes in analogous structures as further described hereinbelow.

According to another specific embodiment, the interaction is to amino acid Phe113 of human p53 via Thr6 of the pCAP 250 or its likes in analogous structures as further described hereinbelow.

According to another specific embodiment, the interaction is to amino acid Trp146 of human p53 via Ser5 of the pCAP 250 or its likes in analogous structures as further described hereinbelow.

According to another specific embodiment, the interaction is to amino acid Ser5 of human p53 via Thr6 of the pCAP 250 or its likes in analogous structures as further described hereinbelow.

According to another specific embodiment, the interaction is to amino acid His8 of human p53 via Thr6 of the pCAP 250 or its likes in analogous structures as further described hereinbelow.

According to another specific embodiment, the interaction is to amino acid Gly112 of human p53 via Ser5 of the pCAP 250 or its likes in analogous structures as further described hereinbelow.

According to another specific embodiment, the interaction is to amino acid Gly112 of human p53 via Thr6 of the pCAP 250 or its likes in analogous structures as further described hereinbelow.

Other suggested positions for interactions on the surface of p53 DBD are listed in FIGS. 6A-C which is considered as part of the specification wherein each possibility represents an independent embodiment.

Other suggested positions for interactions on the surface of p53 DBD are listed in FIGS. 9-14 which is considered as part of the specification wherein each possibility represents an independent embodiment.

Methods of elucidating the amino acids either in the peptide or in the DBD which are critical for the interaction are well known in the art and include, but are not limited to crystallography, as well as the use of computer-based algorithms e.g., AnchorDock (Ben Shimon Structure. 2015 May 5; 23(5):929-40), Virtual crystallographic Calculators V.2. and the like.

According to a specific embodiment, the peptide comprises a consensus motif.

The term “consensus motif” as used herein refers to an amino acid sequence of at least 3 amino acids, 4, 5 or 6 amino acids which may be consecutive or non-consecutive. According to a specific embodiment, the consensus motif is 6 consecutive amino acids long.

According to a specific embodiment, the peptide comprises an amino acid sequence of:

X₁-X₂-X₃-X₄-X₅-X₆ (SEQ ID NO: 53)

wherein,
X₁ and X₅ are a positively charged amino acid;
X₂ is selected from the group consisting of Ser, Thr, Asn, Gln, Pro, Ala and Gly;
X₃ is any amino acid;
X₄ and X₆ are selected from the group consisting of an alpha methyl amino and a beta-breaker amino acid.

According to a specific embodiment, the peptide comprises an amino acid sequence of:

X₁-X₂-X₃-X₄-X₅-X₆ (SEQ ID NO: 54)

wherein,
X₁ and X₅ are selected from the group consisting of His, Arg and Lys;
X₂ is selected from the group consisting of Ser, Thr, Asn, Gln, Pro, Ala and Gly;
X₃, X₄, X₆ is any amino acid.

As used herein “positively charged amino acid” is an amino acid that can be positive (i.e. protonated) at physiological pH.

According to an embodiment, the positively charged amino acid is selected from the group consisting of is, Diaminobutyric acid (Dab), Arg and Lys.

According to a specific embodiment, X₃ is a D-amino acid.

According to a specific embodiment, X₃ is a phosphorylated (e.g phosphoserine) or phosphomimetic thereof (e.g., Glu or Asp).

According to a specific embodiment, X₃ is a non-phosphorylatable amino acid (e.g., Val).

According to a specific embodiment, the X₃ is a non-hydrogen bonding amino acid (e.g. Ala).

According to a specific embodiment, the X₃ is selected from the group consisting of polar uncharged amino acid (e.g., Ser) and a hydrophobic amino acid (e.g. Ile).

According to a specific embodiment, the X₂ is Ser.

According to a specific embodiment, the X₄ and X₆ are selected from the group consisting of Ser, Thr, Pro, Ala and Gly.

According to a specific embodiment, the X₄ is an alpha methyl amino acid or a beta breaker, e.g., Pro, Aib or Ala.

According to a specific embodiment, the X₄ is an alpha methyl amino acid.

According to a specific embodiment, the X₆ is Ala.

According to a specific embodiment, the peptide has the amino acid sequence HSAPHP (SEQ ID NO: 46).

According to a specific embodiment, the peptide comprises at least one additional amino acid (X₇) attached to the C-terminus of said amino acid sequence.

According to a specific embodiment, the at least one additional amino acid is a negatively charged amino acid (i.e., amino acid that is typically negative (i.e. de-protonated) at physiological pH) or a small amino acid (e.g., Gly, Ala, Val).

According to a specific embodiment, the at least one additional amino acid is selected from the group consisting of Asp, Glu, Gly, Ala and Ser.

According to a specific embodiment, the at least one negatively charged amino acid is Asp.

According to a specific embodiment, the at least one additional amino acid comprises two additional amino acids (X₇-X₈) and wherein said X₈ is selected from the group consisting of His, Dab, Asp and Glu.

According to a specific embodiment, the at least one negatively charged amino acid is Asp or two consecutive Asp residues.

According to a specific embodiment, the peptide comprises at least one additional amino acid attached to the N-terminus of said amino acid sequence.

According to a specific embodiment, the peptide comprises at least two additional amino acids attached to the N-terminus of said amino acid sequence.

According to a specific embodiment, the at least one additional amino acid attached to the N-terminus of said amino acid sequence is Arg or two consecutive Arg residues.

Binding of the peptide to the DBD can be determined using any method known in the art, such as a competition assay wherein a soluble DBD is used as a competing agent.

The term “recombinant or synthetic peptide” as used herein refers to a peptide produced by standard biotechnological methods known in the art, such as expression in bacteria or Solid-phase peptide synthesis (SPPS).

According to a specific embodiment, the peptide further comprises a cell penetrating moiety, which can be attached to the N-terminus of the peptide, the C-terminus of the peptide or at both ends of the peptide. It will be appreciated that this moiety can also be bound to the peptide body not via its termini, as long as it doesn't interfere with the binding of the peptide to the DBD. It will be appreciated that this moiety is a heterologous moiety that is not bound to the peptide in nature in the same manner (i.e., position or chemistry).

The term “Permeability” as used herein refers to the ability of an agent or substance to penetrate, pervade, or diffuse through a barrier, membrane, or a skin layer. A “cell permeability” or a “cell-penetration” moiety refers to any molecule known in the art which is able to facilitate or enhance penetration of molecules through membranes.

As used herein the phrase “permeability-enhancing moiety” refers to an agent which enhances translocation of any of the attached peptide across a cell membrane.

Any moiety known in the art to facilitate actively or passively or enhance permeability of compositions into cells may be used for conjugation with the peptide core according to the present invention. Non-limitative examples include: hydrophobic moieties such as fatty acids, steroids and bulky aromatic or aliphatic compounds; moieties which may have cell-membrane receptors or carriers, such as steroids, vitamins and sugars, natural (e.g., positively charged amino acids e.g., Lys or Arg) and non-natural amino acids and proteinaceous moiety e.g., transporter peptides, also referred to as “cell penetrating peptides” or a CPP, poly-Arginine or poly-Lysine, a combination of same or an antibody. According to some embodiments, the proteinaceous moiety is a CPP. According to some embodiments, the proteinaceous moiety is poly-Arginine.

According to some embodiments, the hydrophobic moiety is a lipid moiety or an amino acid moiety. According to some embodiments of the invention, the cell penetrating moiety is a combination of a proteinaceous moiety and a lipid-based moiety (e.g., one from the N terminus and the other from the C-terminus of the peptide).

Cell-Penetrating Peptides (CPPs) are short peptides (<40 amino acids), with the ability to gain access to the interior of almost any cell. They are highly cationic and usually rich in arginine and lysine amino acids. Indeed the present inventors have used positively charged amino acids (on either peptide termini) or poly-cationic amino acids (at least 2 e.g., 2-12) poly-Arg to impart the peptides with cell permeation. They have the exceptional property of carrying into the cells a wide variety of covalently and noncovalently conjugated cargoes such as proteins, oligonucleotides, and even 200 nm liposomes. Therefore, according to additional exemplary embodiment CPPs can be used to transport the peptides to the interior of cells.

TAT (transcription activator from HIV-1), pAntp (also named penetratin, Drosophila antennapedia homeodomain transcription factor) and VP22 (from Herpes Simplex virus) are examples of CPPs that can enter cells in a non-toxic and efficient manner and may be suitable for use with some embodiments of the invention. Protocols for producing CPPs-cargos conjugates and for infecting cells with such conjugates can be found, for example L Theodore et al. [The Journal of Neuroscience, (1995) 15(11): 7158-7167], Fawell S, et al. [Proc Natl Acad Sci USA, (1994) 91:664-668], and Jing Bian et al. [Circulation Research (2007) 100: 1626-1633].

However, the disclosure is not so limited, and any suitable penetrating agent may be used, as known by those of skill in the art.

When the peptides of the present invention are attached to cell penetrating peptides, it is contemplated that the full length peptide is no greater than 50 amino acids, no greater than 40 amino acids, no greater than 35 amino acids, no greater than 30 amino acids, no greater than 25 amino acids, no greater than 22 amino acids, no greater than 20 amino acids, no greater than 15 amino acids, no greater than 12 amino acids, no greater than 10 amino acids, no greater than 9 amino acids, no greater than 8 amino acids, or no greater than 7 amino acids.

Non-limitative examples of non-proteinaceous cell penetrating moieties include: hydrophobic moieties such as lipids, fatty acids, steroids and bulky aromatic or aliphatic compounds; moieties which may have cell-membrane receptors or carriers, such as steroids, vitamins and sugars, nanoparticles and liposomes.

The term “fatty acid moiety” as used herein refers to a part of a fatty acid that exhibits a particular set of chemical and pharmacologic characteristics similar to the corresponding complete fatty acid origin molecule. The term further refers to any molecular species and/or molecular fragment comprising the acyl component of a fatty (carboxylic) acid.

A permeability-enhancing moiety according to the present invention is preferably connected covalently to the peptide sequence via a direct bond or via a linker, to form a peptide conjugate. The permeability-enhancing moiety may be connected to any position in the peptide moiety, directly or through a spacer, preferably to the amino terminus of the peptide. According to certain embodiments, the permeability enhancing moiety is a fatty acid.

The hydrophobic moiety according to the invention may preferably comprise a lipid moiety or an amino acid moiety. According to a specific embodiment the hydrophobic moiety is selected from the group consisting of: phospholipids, steroids, sphingosines, ceramides, octyl-glycine, 2-cyclohexylalanine, benzolylphenylalanine, propionoyl (C₃); butanoyl (C₄); pentanoyl (C₅); caproyl (C₆); heptanoyl (C₇); capryloyl (C₈); nonanoyl (C₉); capryl (C₁₀); undecanoyl (C₁₁); lauroyl (C₁₂); tridecanoyl (C₁₃); myristoyl (C₁₄); pentadecanoyl (C₁₅); palmitoyl (C₁₆); phtanoyl ((CH₃)₄); heptadecanoyl (C₁₇); stearoyl (C₁₈); nonadecanoyl (C₁₉); arachidoyl (C₂₀); heniecosanoyl (C₂₁); behenoyl (C₂₂); trucisanoyl (C₂₃); and lignoceroyl (C₂₄); wherein said hydrophobic moiety is attached to said chimeric polypeptide with amide bonds, sulfhydryls, amines, alcohols, phenolic groups, or carbon-carbon bonds.

Other examples for lipidic moieties which may be used according to the present invention: Lipofectamine, Transfectace, Transfectam, Cytofectin, DMRIE, DLRIE, GAP-DLRIE, DOTAP, DOPE, DMEAP, DODMP, DOPC, DDAB, DOSPA, EDLPC, EDMPC, DPH, TMADPH, CTAB, lysyl-PE, DC-Cho, -alanyl cholesterol; DCGS, DPPES, DCPE, DMAP, DMPE, DOGS, DOHME, DPEPC, Pluronic, Tween, BRIJ, plasmalogen, phosphatidylethanolamine, phosphatidylcholine, glycerol-3-ethylphosphatidylcholine, dimethyl ammonium propane, trimethylammonium propane, diethylammonium propane, triethylammonium propane, dimethyldioctadecylammonium bromide, a sphingolipid, sphingomyelin, a lysolipid, a glycolipid, a sulfatide, a glycosphingolipid, cholesterol, cholesterol ester, cholesterol salt, oil, N-succinyldioleoylphosphatidylethanolamine, 1,2-dioleoyl-sn-glycerol, 1,3-dipalmitoyl-2-succinylglycerol, 1,2-dipalmitoyl-sn-3-succinylglycerol, 1-hexadecyl-2-palmitoylglycerophosphatidylethanolamine, palmitoylhomocystiene, N,N′-Bis (dodecyaminocarbonylmethylene)-N,N′-bis((-N,N,N-trimethylammoniumethyl-aminocarbonylmethylene)ethylenediamine tetraiodide; N,N″-Bis(hexadecylaminocarbonylmethylene)-N,N′,N″-tris((-N,N,N-trimethylammonium-ethylaminocarbonylmethylenediethylenetri amine hexaiodide; N,N′-Bis(dodecylaminocarbonylmethylene)-N,N″-bis((-N,N,N-trimethylammonium ethylaminocarbonylmethylene)cyclohexylene-1,4-diamine tetraiodide; 1,7,7-tetra-((-N,N,N,N-tetramethylammoniumethylamino-carbonylmethylene)-3-hexadecylaminocarbonyl-methylene-1,3,7-triaazaheptane heptaiodide; N,N,N′,N′-tetra((-N,N,N-trimethylammonium-ethylaminocarbonylmethylene)-N′-(1,2-dioleoylglycero-3-phosphoethanolamino carbonylmethylene)diethylenetriam the tetraiodide; dioleoylphosphatidylethanolamine, a fatty acid, a lysolipid, phosphatidylcholine, phosphatidylethanolamine, phosphatidylserine, phosphatidylglycerol, phosphatidylinositol, a sphingolipid, a glycolipid, a glucolipid, a sulfatide, a glycosphingolipid, phosphatidic acid, palmitic acid, stearic acid, arachidonic acid, oleic acid, a lipid bearing a polymer, a lipid bearing a sulfonated saccharide, cholesterol, tocopherol hemisuccinate, a lipid with an ether-linked fatty acid, a lipid with an ester-linked fatty acid, a polymerized lipid, diacetyl phosphate, stearylamine, cardiolipin, a phospholipid with a fatty acid of 6-8 carbons in length, a phospholipid with asymmetric acyl chains, 6-(5-cholesten-3b-yloxy)-1-thio-b-D-galactopyranoside, digalactosyldiglyceride, 6-(5-cholesten-3b-yloxy)hexyl-6-amino-6-deoxy-1-thio-b-D-galactopyranoside, 6-(5-cholesten-3b-yloxy)hexyl-6-amino-6-deoxyl-1-thio-a-D-mannopyranoside, 12-4(7′-diethylamino-coumarin-3-yl)carbonyOmethylamino)-octadecanoic acid; N-[12-(((7′-diethylaminocoumarin-3-yl)carbonyl)methyl-amino) octadecanoyl]-2-aminopalmitic acid; cholesteryl)4′-trimethyl-ammonio)butanoate; N-succinyldioleoyl-phosphatidylethanolamine; 1,2-dioleoyl-sn-glycerol; 1,2-dipalmitoyl-sn-3-succinyl-glycerol; 1,3-dipalmitoyl-2-succinylglycerol, 1-hexadecyl-2-palmitoylglycero-phosphoethanolamine, and palmitoylhomocysteine.

The terms “polypeptide” and “peptide” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated amide bonds (—N(CH3)-CO—), ester bonds (—C(═O)—O—), ketomethylene bonds (—CO—CH2-), sulfinylmethylene bonds (—S(═O)—CH2-), α-aza bonds (—NH—N(R)—CO—), wherein R is any alkyl (e.g., methyl), amine bonds (˜CH2-NH—), sulfide bonds (˜CH2-S—), ethylene bonds (˜CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the peptide chain and even at several (2-3) bonds at the same time.

“Conservative substitution” refers to the substitution of an amino acid in one class by an amino acid of the same class, where a class is defined by common physico-chemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gin, Glu); Class IV (His, Arg, Lys); Class V (He, Leu, Val, Met); and Class VI (Phe, Tyr, Trp). For example, substitution of an Asp for another Class III residue such as Asn, Gin, or Glu, is a conservative substitution.

Other classifications include positive amino acids (Arg, His, Lys), negative amino acids (Asp, Glu), polar uncharged (Ser, Thr, Asn, Gln), hydrophobic side chains (Ala, Val, Ile, Leu, Met, Phe, Tyr, Trp).

“Non-conservative substitution” refers to the substitution of an amino acid in to one class with an amino acid from another class; for example, substitution of an Ala, a Class II residue, with a Class III residue such as Asp, Asn, Glu, or Gin.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr. Other synthetic options are listed hereinbelow in Table 2.

The peptides of some embodiments of the invention may also include one or more modified amino acids or one or more non-amino acid monomers (e.g. fatty acids, complex carbohydrates etc.).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1), and non-conventional or modified amino acids (e.g., synthetic, Table 2) which can be used with some embodiments of the invention.

TABLE 1

| Amino | Three-Letter | One-letter |
|---|---|---|
| Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid | Xaa | X |
| as above | | |

TABLE 2

| Non-conventional amino acid | Code | Non-conventional amino acid | Code |
|---|---|---|---|
| ornithine | Orn | hydroxyproline | Hyp |
| α-aminobutyric acid | Abu | aminonorbomyl-carboxylate | Norb |
| D-alanine | Dala | aminocyclopropane-carboxylate | Cpro |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl)glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylomithine | Dnmorn | L-N-methylomithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyelopentylalanine | Nmcpen |
| α-amino-α-methylbutyrate | Aabu | N-methyl-α-amino-α-methylbutyrate | Nmaabu |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | DmIle | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylomithine | Dmorn | L-α-methylomithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyro sine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobuty rate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2-diphenylethyl)glycine | Nbhm | N-(N-(2,2-diphenylethyl)carbamylmethyl-glycine | Nnbhm |
| N-(3,3-diphenylpropyl)glycine | Nbhe | N-(N-(3,3-diphenylpropyl)carbamylmethyl-glycine | Nnbhe |
| 1-carboxy-1-(2,2-diphenylethylamino)cyclopropane | Nmbc | 1,2,3,4-tetrahydroisoquinoline-3-carboxylic acid | Tic |
| phosphoserine | pSer | phosphothreonine | pThr |
| phosphotyrosine | pTyr | O-methyl-tyrosine | |
| 2-aminoadipic acid | | hydroxylysine | |

The peptides of some embodiments of the invention are preferably utilized in a linear form, although it will be appreciated that in cases where cyclicization does not severely interfere with peptide characteristics, cyclic forms of the peptide can also be utilized.

In order to improve bioavailability, the peptide may comprise at least one D amino acid (e.g., 2-7, 2-6, 2-5, 2-4, 2-3). According to a specific embodiment, all the amino acids in the peptide are D amino acids.

In some embodiments, the peptide is chemically modified.

“Chemically modified” refers to an amino acid that is modified either by natural processes, or by chemical modification techniques which are well known in the art. Among the numerous known modifications, typical, but not exclusive examples include: acetylation, acylation, amidation, ADP-ribosylation, glycosylation, glycosaminoglycanation, GPI anchor formation, covalent attachment of a lipid or lipid derivative, methylation, myristlyation, pegylation, prenylation, phos-phorylation, ubiqutination, or any similar process (see e.g., SEQ ID NOs: 2, 17-19).

According to a specific embodiment, the peptide may comprise C-terminal amidation.

Yet alternatively or additionally the peptide may be conjugated to non-proteinaceous non-toxic moiety such as, but are not limited to, polyethylene glycol (PEG), Polyvinyl pyrrolidone (PVP), poly(styrene comaleic anhydride) (SMA), and divinyl ether and maleic anhydride copolymer (DIVEMA).

It will be appreciated that the peptides of the invention can also utilize peptide homologues which exhibit the desired activity (e.g., reactivation of p53 mutants), also referred to herein as functional equivalents, whereby the activity of the peptide homologue is determined according to methods known in the art such as described herein. Such homologues can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to SEQ ID NO: 53 or 54 or 1 (provided its not the peptides disclosed in WO2015/019318 (e.g., SEQ ID NOs: 286-321).

According to a specific embodiment, the peptide comprises the amino acid sequence or is set forth in SEQ ID NO: 8, 412-464.

According to a specific embodiment, the peptide is selected from the group of sequences of SEQ ID NO: 429, 448, 449, 446 and 462.

In certain embodiments, the peptide at least partially changes the conformation of the mutant p53 protein to a conformation of a wild-type (WT) p53 protein.

Known in the art are antibodies that specifically recognize only wild type p53 proteins. Such antibodies are highly useful in determining whether a certain p53 protein, either wild type or mutant, holds the conformation of a wild type, functional p53 protein. Thus, in certain embodiments, the peptide at least partially changes the conformation of the mutant p53 protein such that the mutant p53 protein is recognized by a monoclonal antibody exclusively directed against a WT p53 protein or against a p53 protein holding a WT p53 protein conformation. In certain embodiments, the monoclonal antibody is Ab1620.

It should be understood that since p53 is expressed from both alleles, the overall content of intra-cellular p53 can be either wild-type (wt/wt), mixture of wt and mutant p53 (wt/mut) or mutant p53 only (when both alleles are mutated (mut/mut), or one allele is deleted (mut/−)). In cancer, the situation is often wt/mut, mut/mut or mut/−. Since p53 acts as a tetramer, mutant p53 proteins may abrogate the activity of wild type p53 proteins, which may exist in the cancer's cells. Therefore, the peptides provided by the present invention are particularly useful in treating cancers in which increasing the level of wild type p53 proteins is not fruitful.

In certain embodiments, the peptide at least partially restores the activity of the mutant p53 protein to at least one of the activities of a WT p53 protein.

As used herein the term “reducing” refers to statistically significantly decreasing a certain phenotype by at least about 10%, 20%, 30%, 40%, 50%, 60%, 70%75%, 80%, 95% or even 100% as compared to a control (e.g., same cell/animal system treated with a control vehicle or non-treated at all) under the same assay conditions. As used herein the term “increasing” or “improving” refers to statistically significantly increasing a certain phenotype by at least about 10%, 20%, 30%, 40%, 50%, 60%, 70%, 75%, 80%, 95% or even 100% as compared to a control (e.g., same cell/animal system treated with a control vehicle or non-treated at all) under the same assay conditions.

The term “cells expressing the mutant p53 protein” as used herein refers to cells which express from at least one allele a mutant p53 protein. In certain embodiments, the term “cells expressing the mutant p53 protein” is interchangeable with “cancer cells”.

The term “pro-apoptotic genes” refers to a gene, or a multitude of genes, involved in apoptosis, either directly (such as certain caspases) or indirectly (for example, as part of a signal transduction cascade).

In certain embodiments, the activity is reducing viability of cells expressing the mutant p53 protein. In certain embodiments, the activity is promoting apoptosis of cells expressing the mutant p53 protein. In certain embodiments, the activity is activating pro-apoptotic genes of cells expressing said mutant p53 protein. In certain embodiments, the pro-apoptotic genes are selected from the group consisting of CD95, Bax, DR4, DR5, PUMA, NOXA, Bid, 53AIP1 and PERP. Each possibility represents a separate embodiment of the invention.

In certain embodiments, the activity is binding to a p53 consensus DNA binding element in cells expressing the mutant p53 protein. In certain embodiments, the consensus DNA binding element comprises or consists the nucleotides sequence set forth in SEQ ID NOs: 55 and 56.

Methods of monitoring cellular changes induced by the any of the peptides of the present invention are known in the art and include for example, the MTT test which is based on the selective ability of living cells to reduce the yellow salt MTT (3-(4, 5-dimethylthiazolyl-2)-2, 5-diphenyltetrazolium bromide) (Sigma, Aldrich St Louis, Mo., USA) to a purple-blue insoluble formazan precipitate; the BrDu assay [Cell Proliferation ELISA BrdU colorimetric kit (Roche, Mannheim, Germany]; the TUNEL assay [Roche, Mannheim, Germany]; the Annexin V assay [ApoAlert® Annexin V Apoptosis Kit (Clontech Laboratories, Inc., CA, USA)]; the Senescence associated-n-galactosidase assay (Dimri G P, Lee X, et al. 1995. A biomarker that identifies senescent human cells in culture and in aging skin in vivo. Proc Natl Acad Sci USA 92:9363-9367); as well as various RNA and protein detection methods (which detect level of expression and/or activity) which are further described herein below.

In certain embodiments, the binding results in at least partial activation of an endogenous p53 target gene. In certain embodiments, the endogenous target gene is selected from the group consisting of p21, MDM2 and PUMA. Each possibility represents a separate embodiment of the invention.

In certain embodiments, the mutant p53 protein is of a different conformation than a WT p53 protein. In certain embodiments, the mutant p53 protein is at least partly inactive compared to a WT p53 protein.

In certain embodiments, the mutant p53 protein is not recognized by a monoclonal antibody directed against a WT p53 protein. In certain embodiments, the mutant p53 protein, upon binding to the peptide, is recognized by a monoclonal antibody directed against a WT p53 protein. In certain embodiments, the monoclonal antibody is Ab1620.

In some embodiments, the reactivating peptide can reactivate a Mut-p53 to have structural properties, biochemical properties, physiological properties and/or functional properties similar to or identical to a WT p53 protein.

According to some embodiments, there are provided Mut-p53 reactivating peptides, wherein the peptides are in the length of about 3-25 amino acids. In some embodiments, the Mut-p53 reactivating peptides are in the length of about 4-15 amino acids. In some embodiments, the Mut-p53 reactivating peptides are in the length of about 7-12 amino acids. In some embodiments, the Mut-p53 reactivating peptides are in the length of 7 amino acids. In some embodiments, the Mut-p53 reactivating peptides are in the length of 12 amino acids. Each possibility represents a separate embodiment of the invention.

Other peptide lengths are recited throughout the application. Each possibility represents a separate embodiment of the invention.

According to some embodiments, a Mut-p53 reactivating peptide can affect Mut-p53 such that it can trans-activates a reporter gene (such as Luciferase) having WT p53 binding element in its promoter. In some embodiments the transactivation of the reporter gene may be performed in vitro (for example, in a test tube or well), or in-vivo in a cell, harboring the reporter gene construct.

According to some embodiments, a Mut-p53 reactivating peptide can bind to the DNA binding Domain (DBD) of a mutated p53. In some embodiments, the mutated p53 harbors a mutation in its DNA binding domain (DBD).

The term “pharmaceutical composition” as used herein refers to any composition comprising at least one pharmaceutically active ingredient.

The term “associated with a mutant p53 protein” as used herein refers to any disease, disorder or condition which is caused by a mutant p53 protein or its progression relates to the presence of a mutant p53 protein in a cell or an organ.

It should be understood that since p53 is expressed from both alleles, the overall content of intra-cellular p53 can be either wild-type (wt/wt), mixture of wt and mutant p53 (wt/mut) or mutant p53 only (when both alleles are mutated (mut/mut), or one allele is deleted (mut/−)). In cancer, the situation is often wt/mut, mut/mut or mut/−. Since p53 acts as a tetramer, mutant p53 proteins may abrogate the activity of wild type p53 proteins, which do exist in the cancer's cells. Therefore, the peptides provided by the present invention are particularly useful in treating cancers. Of note, the cell may have more than two p53 alleles at least one of which being of mutant p53.

The term “therapeutically effective amount” as used herein refers to an amount of a composition containing a peptide according to the present invention that is sufficient to reduce, decrease, and/or inhibit a disease, disorder or condition in an individual.

According to an aspect of the invention there is provided a method of treating a disease, disorder or condition associated with a mutant p53 protein, comprising administering to a subject in need thereof a therapeutically effective amount of the isolated peptide as described herein (e.g., SEQ ID NO: 8, 412-464), thereby treating said disease, disorder or condition.

According to an aspect of the invention there is provided a method of treating a disease, disorder or condition associated with a mutant p53 protein, comprising administering to a subject in need thereof a therapeutically effective amount of an isolated peptide comprising an amino acid sequence having a space and configuration that allow binding of the peptide to the DNA Binding Domain (DBD) of p53 in the same mode as pCAP 250 (SEQ ID NO: 1) binds said DBD, wherein said peptide at least partially reactivates a mutant p53 protein and wherein said therapeutically effective amount is 0.01-0.3 mg/kg per day or 0.01-0.2 mg/kg per day (e.g., 0.01-0.35 mg/kg per day, 0.01-0.35 mg/kg per day, 0.01-0.15 mg/kg per day, 0.01-0.1 mg/kg per day, 0.01-0.095 mg/kg per day, 0.01-0.09 mg/kg per day, 0.01-0.085 mg/kg per day, 0.01-0.08 mg/kg per day, 0.01-0.075 mg/kg per day, 0.01-0.07 mg/kg per day, 0.01-0.065 mg/kg per day, 0.01-0.06 mg/kg per day, 0.01-0.055 mg/kg per day, 0.01-0.05 mg/kg per day, 0.01-0.45 mg/kg per day, 0.01-0.04 mg/kg per day, 0.01-0.035 mg/kg per day, 0.01-0.03 mg/kg per day), thereby treating said disease, disorder or condition.

As referred to herein, the term “treating a disease” or “treating a condition” is directed to administering a composition, which includes at least one agent, effective to ameliorate symptoms associated with a disease, to lessen the severity or cure the disease, or to prevent the disease from occurring in a subject. Administration may include any administration route. In some embodiments, the disease is a disease that is caused by or related to the presence of a mutated p53 in a cell, tissue, organ, body, and the like. In some embodiments, the disease is cancer. In some embodiments, the cancer is selected from the group consisting of breast cancer, colon cancer, ovarian cancer and lung cancer.

In some embodiments, the cancer is a metastatic cancer.

In some embodiments, the cancer is a metastatic breast cancer, metastatic colon cancer, metastatic ovarian cancer or metastatic lung cancer.

Each possibility represents a separate embodiment of the invention. In some embodiments, the subject is a mammal, such as a human. In some embodiments, the subject is a mammal animal. In some embodiments, the subject is a non-mammal animal. In some embodiments the subject is diagnosed with the disease, condition or disorder.

In some embodiments, cancer is adrenocortical carcinoma, anal cancer, bladder cancer, brain tumor, brain stem glioma, brain tumor, cerebellar astrocytoma, cerebral astrocytoma, ependymoma, medulloblastoma, supratentorial primitive neuroectodermal, pineal tumors, hypothalamic glioma, breast cancer, carcinoid tumor, carcinoma, cervical to cancer, colon cancer, endometrial cancer, esophageal cancer, extrahepatic bile duct cancer, ewings family of tumors (pnet), extracranial germ cell tumor, eye cancer, intraocular melanoma, gallbladder cancer, gastric cancer, germ cell tumor, extragonadal, gestational trophoblastic tumor, head and neck cancer, hypopharyngeal cancer, islet cell carcinoma, laryngeal cancer, leukemia, acute lymphoblastic, leukemia, oral cavity cancer, liver cancer, lung cancer, small cell, lymphoma, AIDS-related, lymphoma, central nervous system (primary), lymphoma, cutaneous T-cell, lymphoma, hodgkin's disease, non-hodgkin's disease, malignant mesothelioma, melanoma, merkel cell carcinoma, metasatic squamous carcinoma, multiple myeloma, plasma cell neoplasms, mycosis fungoides, myelodysplastic syndrome, myeloproliferative disorders, nasopharyngeal cancer, neuroblastoma, oropharyngeal cancer, osteosarcoma, ovarian epithelial cancer, ovarian germ cell tumor, ovarian low malignant potential tumor, pancreatic cancer, exocrine, pancreatic cancer, islet cell carcinoma, paranasal sinus and nasal cavity cancer, parathyroid cancer, penile cancer, pheochromocytoma cancer, pituitary cancer, plasma cell neoplasm, prostate cancer, rhabdomyosarcoma, rectal cancer, renal cell cancer, salivary gland cancer, sezary syndrome, skin cancer, cutaneous T-cell lymphoma, skin cancer, kaposi's sarcoma, skin cancer, melanoma, small intestine cancer, soft tissue sarcoma, soft tissue sarcoma, testicular cancer, thymoma, malignant, thyroid cancer, urethral cancer, uterine cancer, sarcoma, unusual cancer of childhood, vaginal cancer, vulvar cancer, or wilms' tumor.

In some embodiments, the cancer is a lung cancer.

In some embodiments, the cancer is an ovarian cancer.

In some embodiments, the cancer is a triple negative breast cancer.

In some embodiments, the cancer is a metastatic lung cancer.

In some embodiments, the cancer is a metastatic ovarian cancer.

In some embodiments, the cancer is a metastatic triple negative breast cancer.

In some embodiments, cancer is a non-solid tumor such as a blood cancer. In another embodiment, a non-solid tumor or blood cancer is leukemia or lymphoma. In another embodiment, a non-solid tumor or blood cancer is acute lymphoblastic leukemia (ALL). In another embodiment, a non-solid tumor or blood cancer is acute myelogenous leukemia (AML). In another embodiment, a non-solid tumor or blood cancer is chronic lymphocytic leukemia (CLL). In another embodiment, a non-solid tumor or blood cancer is small lymphocytic lymphoma (SLL). In another embodiment, a non-solid tumor or blood cancer is chronic myelogenous leukemia (CML). In another embodiment, a non-solid tumor or blood cancer is acute monocytic leukemia (AMOL). In another embodiment, a non-solid tumor or blood cancer is Hodgkin's lymphomas (any of the four subtypes). In another embodiment, a non-solid tumor or blood cancer is Non-Hodgkin's lymphomas (any of the subtypes). In another embodiment, a non-solid tumor or blood cancer is myeloid leukemia.

For use in the methods of the invention, the reactivating peptides may be formulated in a conventional manner using one or more pharmaceutically acceptable carriers, stabilizers or excipients (vehicles) to form a pharmaceutical composition as is known in the art, in particular with respect to protein active agents. Carrier(s) are “acceptable” in the sense of being compatible with the other ingredients of the composition and not deleterious to the recipient thereof. Suitable carriers typically include physiological saline or ethanol polyols such as glycerol or propylene glycol. The reactivating peptides may be formulated as neutral or salt forms.

Pharmaceutically acceptable salts include the acid addition salts (formed with free amino groups) and which are formed with inorganic acids such as hydrochloric or phosphoric acids, or such organic acids such as acetic, oxalic, tartaric and maleic. Salts formed with the free carboxyl groups may also be derived from inorganic bases such as sodium, potassium, ammonium, calcium, or ferric hydroxides, and organic bases as isopropylamine, trimethylamine, 2-ethylamino ethanol, histidine and procaine.

The compositions may be suitably formulated for intravenous, intramuscular, subcutaneous, or intraperitoneal administration and conveniently comprise sterile aqueous solutions of the reactivating peptides, which are preferably isotonic with the blood of the recipient. Such formulations are typically prepared by dissolving solid active ingredient in water containing physiologically compatible substances such as sodium chloride, glycine, and the like, and having a buffered pH compatible with physiological conditions to produce an aqueous solution, and rendering said solution sterile. These may be prepared in unit or multi-dose containers, for example, sealed ampoules or vials.

The compositions may incorporate a stabilizer, such as for example polyethylene glycol, proteins, saccharides (for example trehalose), amino acids, inorganic acids and admixtures thereof. Stabilizers are used in aqueous solutions at the appropriate concentration and pH. The pH of the aqueous solution is adjusted to be within the range of 5.0-9.0, preferably within the range of 6-8. In formulating the reactivating peptides, anti-adsorption agent may be used. Other suitable excipients may typically include an antioxidant such as ascorbic acid.

The compositions may be formulated as controlled release preparations which may be achieved through the use of polymer to complex or absorb the proteins. Appropriate polymers for controlled release formulations include for example polyester, polyamino acids, polyvinyl, pyrrolidone, ethylenevinylacetate, and methylcellulose. Another possible method for controlled release is to incorporate the reactivating peptides into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, by coacervation techniques or by interfacial polymerization, for example, hydroxymethylcellulose or gelatin-microcapsules and poly(methylmethacylate) microcapsules, respectively, or in colloidal drug delivery systems, for example, liposomes, albumin microspheres, microemulsions, nanoparticles, and nanocapsules or in macroemulsions.

In some embodiments, the reactivating peptides of the invention may be formulated in peroral or oral compositions and in some embodiments, comprise liquid solutions, emulsions, suspensions, and the like. In some embodiments, pharmaceutically-acceptable carriers suitable for preparation of such compositions are well known in the art. In some embodiments, liquid oral compositions comprise from about 0.001% to about 0.9% of reactivating peptides, or in another embodiment, from about 0.01% to about 10%.

In some embodiments, compositions for use in the methods of this invention comprise solutions or emulsions, which in some embodiments are aqueous solutions or emulsions comprising a safe and effective amount of a reactivating peptide and optionally, other compounds, intended for topical intranasal administration.

In some embodiments, injectable solutions of the invention are formulated in aqueous solutions. In one embodiment, injectable solutions of the invention are formulated in physiologically compatible buffers such as Hank's solution, Ringer's solution, or physiological salt buffer. In some embodiments, for transmucosal administration, penetrants appropriate to the barrier to be permeated are used in the formulation. Such penetrants are generally known in the art.

In one embodiment, the preparations described herein are formulated for parenteral administration, e.g., by bolus injection or continuous infusion. In some embodiments, formulations for injection are presented in unit dosage form, e.g., in ampoules or in multidose containers with optionally, an added preservative. In some embodiments, compositions are suspensions, solutions or emulsions in oily or aqueous vehicles, and contain formulatory agents such as suspending, stabilizing and/or dispersing agents.

The reactivating peptides of the invention may be administered by any suitable administration route, selected from oral, topical, transdermal or parenteral administration. According to some embodiments the route of administration is via topical application selected from dermal, vaginal, rectal, inhalation, intranasal, ocular, auricular and buccal. According to some embodiments the route of administration is via parenteral injection. In various embodiments, the step of administering is carried out by a parenteral route selected from the group consisting of intravenous, intramuscular, subcutaneous, intradermal, intraperitoneal, intraarterial, intracerebral, intracerebroventricular, intraosseus and intrathecal. For example, the reactivating peptides may be administered systemically, for example, by parenteral routes, such as, intraperitoneal (i.p.), intravenous (i.v.), subcutaneous, or intramuscular routes. The reactivating peptides of the invention and/or any optional additional agent may be administered systemically, for example, by intranasal administration. The reactivating peptides of the invention and/or any optional additional agent may be administered systemically, for example, by oral administration, by using specific compositions or formulations capable of providing oral bioavailability to proteins. The reactivating peptides of the invention and/or any optional additional agent may be administered locally.

According to a specific embodiment, administering comprises subcutaneous administering.

Alternatively or additionally, according to a specific embodiment, administering comprises continuous infusion.

Thus the reactivating peptides (e.g., SEQ ID NO: 1, 8, or 412-464 or 429, 448, 449, 446, 462) can also be delivered by slow-release delivery systems, pumps, and other known delivery systems for continuous infusion for example in the following doses e.g., 0.01-0.3 mg/kg per day, 0.01-0.15 mg/kg per day, 0.01-0.1 mg/kg per day, 0.01-0.095 mg/kg per day, 0.01-0.09 mg/kg per day, 0.01-0.085 mg/kg per day, 0.01-0.08 mg/kg per day, 0.01-0.075 mg/kg per day, 0.01-0.07 mg/kg per day, 0.01-0.065 mg/kg per day, 0.01-0.06 mg/kg per day, 0.01-0.055 mg/kg per day, 0.01-0.05 mg/kg per day, 0.01-0.45 mg/kg per day, 0.01-0.04 mg/kg per day, 0.01-0.035 mg/kg per day, 0.01-0.03 mg/kg per day). Dosing regimens may be varied to provide the desired circulating levels of particular reactivating peptides based on its pharmacokinetics. Thus, doses are calculated so that the desired circulating level of therapeutic agent is maintained.

Typically, the effective dose is determined by the activity of the reactivating peptides and the condition of the subject, as well as the body weight or surface area of the subject to be treated. The size of the dose and the dosing regime is also determined by the existence, nature, and extent of any adverse side effects that accompany the administration of the reactivating peptides in the particular subject.

In some embodiments, there is provided a kit for treating or preventing a p53 related condition. In some embodiments, the kit comprises a container (such as a vial) comprising a Mut-p53 reactivating peptide in a suitable buffer and instructions for use for administration of the reactivating peptide.

It is suggested that the efficacy of treatment with the peptides of the invention may be augmented when combined with gold standard treatments (e.g., anti-cancer therapy). Thus, the peptide can be used to treat diseases or conditions associated with p53 (as described hereinabove) alone or in combination with other established or experimental therapeutic regimen for such disorders. It will be appreciated that treatment with additional therapeutic methods or compositions has the potential to significantly reduce the effective clinical doses of such treatments, thereby reducing the often devastating negative side effects and high cost of the treatment.

Therapeutic regimen for treatment of cancer suitable for combination with the peptides of some embodiments of the invention or polynucleotide encoding same include, but are not limited to chemotherapy, radiotherapy, phototherapy and photodynamic therapy, surgery, nutritional therapy, ablative therapy, combined radiotherapy and chemotherapy, brachiotherapy, proton beam therapy, immunotherapy, cellular therapy and photon beam radiosurgical therapy. According to a specific embodiment, the chometherapy is platinum-based.

Anti-Cancer Drugs

Anti-cancer drugs that can be co-administered with the compounds of the invention include, but are not limited to Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

According to another aspect of the invention there is provided a method of treating a disease, disorder or condition associated with a mutant p53 protein, comprising administering to a subject in need thereof a therapeutically effective amount of a platin-based chemotherapy and an isolated peptide comprising an amino acid sequence having a space and configuration that allow binding of the peptide to the DNA Binding Domain (DBD) of p53 in the same mode as pCAP 250 (SEQ ID NO: 1) binds said DBD (e.g., SEQ ID NO: 1, 8, 412-464, 429, 448, 449, 446, 462), wherein said peptide at least partially reactivates a mutant p53 protein, thereby treating said disease, disorder or condition.

Specific examples of platinum-based chemotherapies include, but are not limited to, cisplatin, the first to be developed, carboplatin, a second-generation platinum-based antineoplastic agent, oxaliplatin, satraplatin, picoplatin, Nedaplatin, Triplatin, Lipoplatin, a liposomal version of cisplatin.

Kits and articles or manufacture for effecting combination treatments as described herein (e.g., the peptide together with platinum-based chemotherapy) are also contemplated herein.

It will be appreciated that a peptide comprising the amino acid sequence selected from the group consisting of 59-382 can also be implemented in the above-described methods.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Experimental Procedures

Crystal Violet Viability Assay

Cells were cultured in 96 wells plates with 2500-4000 cells/well. Serial dilutions of different peptides were added and the plates incubated for additional 48 h at 37° C. Then medium was removed and cell viability was determined by staining the cells with crystal violet (0.05%) in methanol/PBS (1:5, v/v), for 10 min, followed by 3 washes with PBS. 10% acetic acid was added to each well for 10 min. OD was determined at 595 nm.

ChIP Analysis

Cells were cross-linked with formaldehyde (1% final concentration) at room temperature for 10 min. The formaldehyde was neutralized with glycine 0.25M for 5 min. Cells were washed twice with 10 ml of ice-cold PBS and harvested by scraping. Eventually, cells were resuspended in 0.3 ml of lysis buffer (1% SDS, 10 mM EDTA, 50 mM Tris-HCl, pH 8.1, protease inhibitor cocktail) and sonicated for 6 min in sonication bath followed by centrifugation for 10 min on ice to produce 200-500 bp fragments. Supernatants were collected and diluted 10 times in the ChIP dilution buffer (1% Triton X-100, 2 mM EDTA, 150 mM NaCl, 20 mM Tris-HCl, pH 8.1) followed by immuno-clearing with 40p1 of pre-blocked protein A-sepharose with 2 μg sheared salmon sperm DNA and 10 μg BSA for 2 hour at 4° C. Immuno-precipitation was performed overnight at 4° C. with specific ap53 or aRNApolII poly clonal antibodies. Following immuno-precipitation, 40 μl protein A-Sepharose were added and further incubated for another 1 hr. Precipitates were sequentially washed in TSE I (0.1% SDS, 1% Triton X-100, 2 mM EDTA, 20 mM Tris-HCl, pH 8.1, 150 mM NaCl), TSE II (500 mM NaCl), and buffer III (0.25 M LiCl, 1% NP-40, 1% deoxycholate, 1 mM EDTA, 10 mM Tris-HCl, pH 8.1). Precipitates were washed three times with TE buffer and extracted twice with 1% SDS, 0.1 M NaHCO₃. Eluates were pooled and heated at 65° C. for overnight to reverse the formaldehyde cross-linking. DNA fragments were purified with a QIAquick Spin Kit (Qiagen, CA). Immuno-precipitation reactions were performed in triplicate. Beads only served as a non-specific control. Quantitative analysis of the active and repressive histone marks in the ChIP products from clones were assessed by quantitative RT-PCR. In order to normalize the efficiency of immunoprecipitation (IP), the normalization of chromatin IP was done using specific primers for necdin promoter region and 5′ region.

CRISPR p53 Knockout

Plasmid #42230, containing a TP53 exon 3 single guide RNA (sgRNA), was from Addgene. ES2 cells were transfected using jetPEI reagent (Polyplus) according to the manufacturer's protocol. After 48 hours, cells were seeded in a 96 well plate as single cell clones. Single cell clones were expanded and their p53 status was examined by Western blot analysis, using the DO-1 anti-p53 antibody.

sgRNA Sequences:

(SEQ ID NO: 47)
F: 5′-CACCGCCATTGTTCAATATCGTCCG-3′
(SEQ ID NO: 48)
R: 5′-AACCGGACGATATTGAACAATGG-3′

Preclinical Testing of Peptides

Mice (6 weeks Athymic nude) were injected subcutaneously with 2×10⁵-10⁶ cells into each femur. All the cell lines employed in these experiments stably express a luciferase reporter gene to enable monitoring of tumor growth by live imaging. 4-18 days later, when tumors reached visible size, the mice were randomly divided into several groups: a control group, treated with either a single control peptide, and groups treated with effective peptide, either a single peptide. Peptides were administered either by intratumoral injection of 10 μg peptide per tumor in 40p1 PBS, three times a week or by Alzet mini pumps 0.8 mg for two weeks. Tumor growth over time was measured by live imaging, using the IVIS2000 system. Exposure time was calibrated to 20 seconds. 16 images were taken over 8 minutes and peak luminescence values were taken for each tumor. Experiments were conducted until tumors reached maximal allowed size of 1 cm³, at which mice were sacrificed and tumors extracted, measured and weighed.

RT-PCR

RNA was obtained using Macherey-Nagel NucleoSpin RNA II Kit on cells pellet according to the manufacturer's protocol. Aliquots of 0.4-1 μg were reverse transcribed using Bio-RT 2000 (Bio-Lab) and random hexamer primers. QRT-PCR was performed on an ABI 7300 instrument (Applied Biosystems) using SYBR Green FastMix ROX (Quanta). RT-PCR primers (All primers sequences are presented 5′ to 3′):

Primers List

| | Forward primer/ | Reverse primer/ |
|---|---|---|
| Gene | (SEQ ID NO: 20-31) | (SEQ ID NO: 32-43) |
| p53 | CCCAAGCAATGGATGATTTGA | GGCATTCTGGGAGCTTCATCT |
| p21 | GGCAGACCAGCATGACAGATT | GCGGATTAGGGCTTCCTCTT |
| PUMA | GACCTCAACGCACAGTACGAG | AGGAGTCCCATGATGAGATTGT |
| MDM2 | AGGCAAATGTGCAATACCAACA | GGTTA CAGCACCATCAGTAGGTACAG |
| CD95 | ACTGTGACCCTTGCACCAAAT | GCCACCCCAAGTTAGATCTGG |
| Btg2 | AGGCACTCACAGAGCACTACAAAC | GCCCTTGGACGGCTTTTC |
| GAPDH | ACCCACTCCTCCACCTTTGA | CTGTTGCTGTAGCCAAATTCGT |
| p21 (ChIP) | GTGGCTCTGATTGGCTTTCTG | CTTGGGCTGCCTGTTTTCAG |
| PUMA (ChIP) | GCGAGACTGTGGCCTTGTGTC | ACTTTGTGGACCCTGGAACG |
| MDM2 (ChIP) | GGTTGACTCAGCTTTTCCTCTTG | TATTTAAACCATGCATTTTCC |
| CD95 (ChIP) | GGATAATTAGACGTACGTGGGC | GGACAATTGACAAAATCAGTATC |
| GAPDH (ChIP) | GTATTCCCCCAGGTTTACAT | AGGAGTGAGTGGAAGACAGAA |

NMR

Purified 15N labeled p53 core domain 1 ml 4004 (aa 94-296) was dialyzed against 1 L of NMR buffer —(157.5 mM sodium phosphate buffer containing 52.5 mM NaCl and 2.625 mM DTT pH 7.2) for 48 h, buffer was changed and sample was dialyzed against 1 L of NMR buffer for an additional 24-48 hour (72 hour in total). 0.5 ml of the sample was subjected to high-resolution NMR. NMR analysis was carried out at the Weizmann institute of science.

Two-dimensional 1H-15N Heteronuclear Single Quantum Coherence (HSQC) spectra of 15N-p53 by itself and when complexed with the peptides as indicated, were recorded at 293 K. Spectra were acquired on a Bruker AVIII-800 NMR spectrometer equipped with a 5 mm inverse detection triple resonance CryoProbe (TCI). Solvent suppression was achieved using WATERGATE sequence.

### Example 1

### pCAP-250 Synergizes with Cisplatin in Reducing Viability of ES2 Ovarian Cancer Cells

ES2 Cells were cultured in 96 wells plates with 3000 cells/well. Serial dilutions of pCAP-250 were added either alone or together with 1 μg/ml of cisplatin and the plates incubated for additional 48 h at 37° C. Then medium was removed and cell viability was determined by staining the cells with crystal violet (0.05%) in methanol/PBS (1:5, v/v), for 10 min, followed by 3 washes with PBS. 10% acetic acid was added to each well for 10 min. OD was determined at 595 nm. The viability of ES2 cells treated with 1 μg/ml was 39%. The IC50 for pCAP-250 was estimated at 3.2 μM and in combination with cisplatin the IC50 for pCAP-250 was estimated at 1.9 μM indicating a synergistic effect between the two compounds.

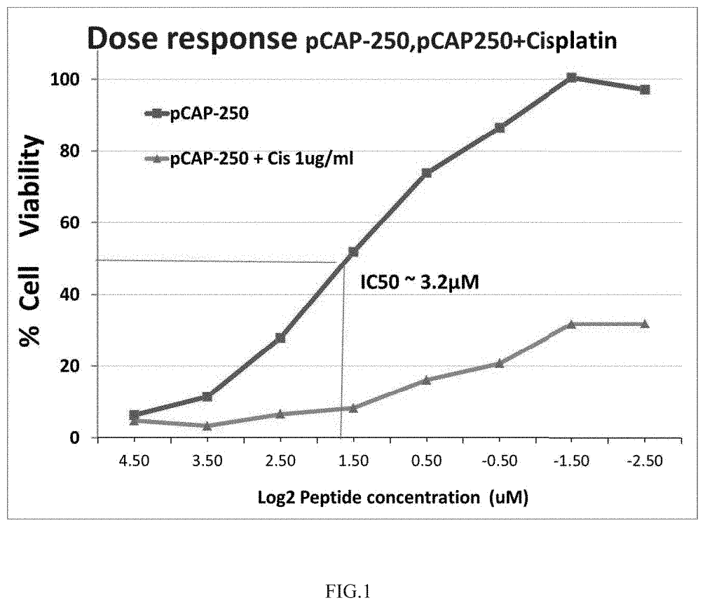

FIG. 1 shows the results of the experiment. Evidently, the viability of the cancer cells reduced significantly in the presence of pCAP 250. A synergy is envisaged by the combined treatment of pCAP 250 with platinum-based chemotherapy.

### Example 2

### Characterizing the Activity of pCAP-250 and Different Derivatives

Cells, ES2 Con expressing endogenous mp53S241F, and ES2 KO cells in which p53 was stably knocked out using CRISPR/Cas9 (ES2 p53KO), to control for specificity for mutp53 were cultured in 96 wells plates with 3000 cells/well. The indicated peptides were added at a concentration of 8 μg/ml and the plates incubated for additional 48 h at 37° C. Then medium was removed and cell viability was determined by staining the cells with crystal violet (0.05%) in methanol/PBS (1:5, v/v), for 10 min, followed by 3 washes with PBS. 10% acetic acid was added to each well for 10 min. OD was determined at 595 nm.

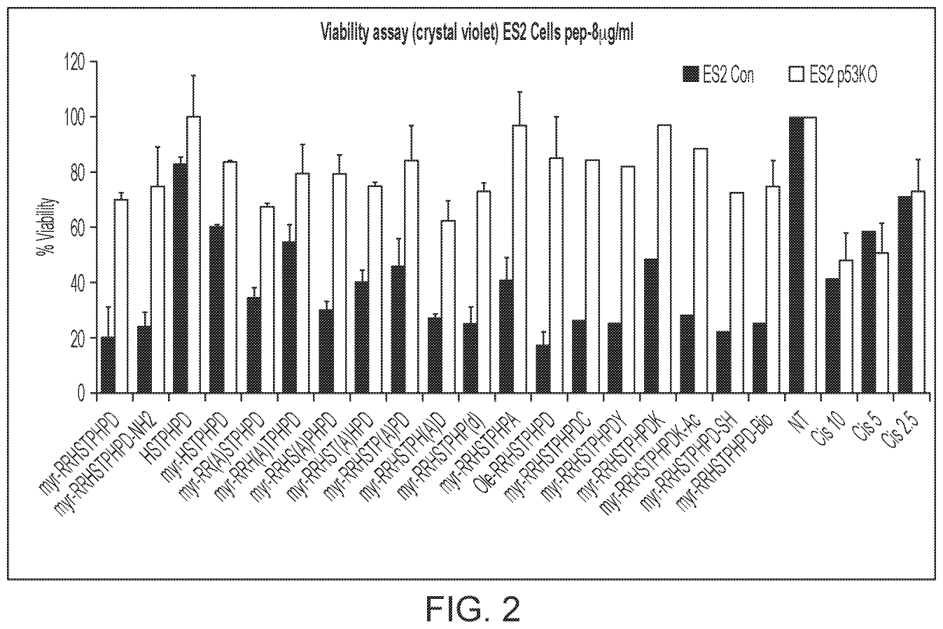

FIG. 2 shows the difference in the effect of a particular peptide for ES2 Con compared to ES KO indicates specificity of peptide to mutp53 expression. Several peptide derivatives in which amino acids that were substituted to Alanine (Serine and Histidine for example) showed a decreased effect on ES2 Con cells indicating the importance of these amino acids for peptide efficacy.

The results were further augmented in an affinity binding assay as described below.

### Example 3

### pCAP 250 Binding to p53 DBD

FIGS. 3A-K show microscale thermophoresis analysis for the binding of fluorescently labeled WTp53DBD and pCAP-250. Experiment was performed according to manufacturer instructions; 10 serial dilutions of pCAP-250 were prepared, labeled protein was added to each peptide sample and loaded to capillaries. The samples were analyzed for movement of fluorescent wtp53DBD in temperature gradient with different concentrations of pCAP-250. Microscale thermophoresis analysis results are presented as a curve obtained from manufacturer data analysis software.

### Example 4

### Pharmacokinetic Study—pCAP 250 Administration Mode and Half Life in Plasma

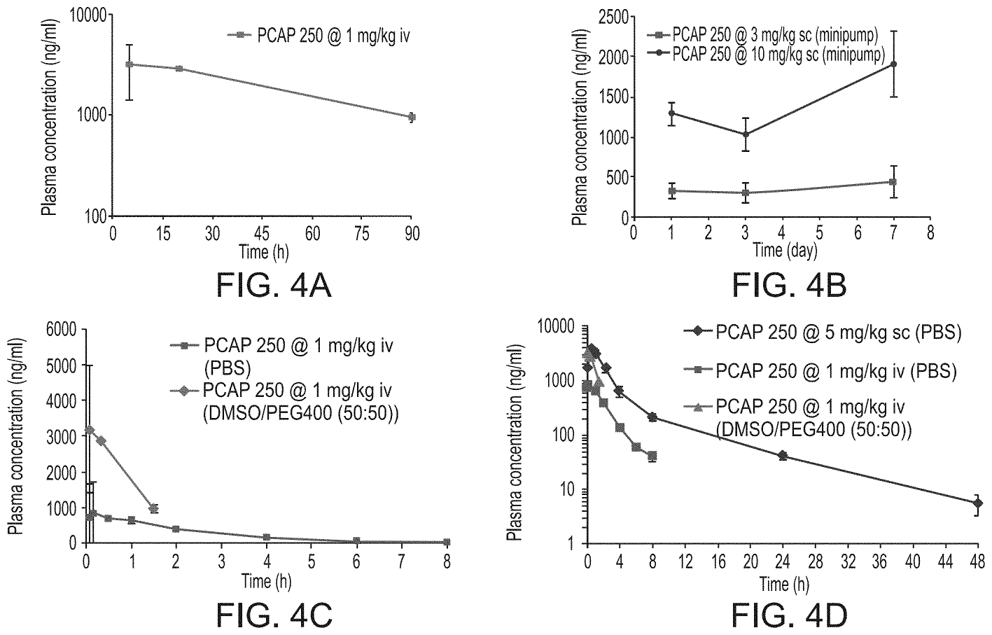

The results of FIGS. 4A-D show that pCAP 250 (SEQ ID NO: 1) has a plasma half-life of 0.8-1.8 hours when administered intravenously. The results further show that pCAP 250 has a plasma half-life of 3-8 hours when administered subcutaneously.

### Example 5

### In-Vivo Effect of pCAP-250 Peptide in a Mouse Xenograft Model

FIGS. 5A-D show that pCAP 250 (SEQ ID NO: 1) when administered by intratumoral injections at dose 0.4 mg/kg 3 times a week has a significant effect on tumor development of ES2 cells in ovarian cancer xenograft model. Further shown is that pCAP 250, when administered subcutaneously by Alzet minipumps, a dose of 2.3 mg/kg per day has a significant effect on tumor development of ES2 cells in ovarian cancer xenograft model.

### Example 6

### Anti-Cancer Activity of pCAP 250 Peptide Variants as Determined by In Vitro Cell Viability Assay

TABLE 3
list of 53 pCAP-250 peptide variants.

| SEQ | pCAP | Peptide |
|---|---|---|
| ID NO: | number | sequnce |
| 412 | 483 | myr-RRHSTPHPGE |
| 413 | 485 | myr-RRHSTPHPSE |
| 414 | 488 | myr-RRHSTPHPAD |
| 415 | 489 | myr-RRHSTPHPAE |
| 416 | 504 | myr-RRHSSPHPD |
| 417 | 505 | myr-RRHSVPHPD |
| 418 | 507 | mvr-RRHSCPHPD |
| 419 | 513 | myr-RRHSePHPD |
| 420 | 514 | myr-RRHStPHPD |
| 421 | 515 | myr-RRHSsPHPD |
| 422 | 516 | myr-RRHSvPHPD |
| 423 | 518 | myr-RR(L-DAB)STPHPD |
| 424 | 519 | myr-RRHSIP(L-DAB)PD |
| 425 | 530 | myr-RRHSTPHPDD-ch3 |
| 426 | 541 | myr-RRHSTPHAD |
| 427 | 551 | myr-RRHSKPHPD |
| 428 | 552 | myr-RRHSSP(L-DAB)PD |
| 429 | 553 | myr-RRHSvP(L-DAB)PD |
| 430 | 554 | myr-RRHSTP(L-DAB)AD |
| 431 | 590 | myr-RRHSsP(L-DAB)PD |
| 432 | 594 | myr-RRHSKPHPDD-NH2 |
| 433 | 595 | myr-RR(L-DAB)STP(L-DAB)PD |
| 434 | 596 | myr-RRHSKP(L-DAB)PD |
| 435 | 597 | myr-RR(L-DAB)SKPHPD |
| 436 | 598 | myr-RR(L-DAB)SKP(L-DAB)PD |
| 437 | 599 | myr-RRHSKPHAD |
| 438 | 600 | myr-RRHSKPHASE |
| 439 | 601 | myr-RRHSKPHPSE |
| 440 | 602 | myr-RR(L-DAB)SsP(L-DAB)PD |
| 441 | 603 | myr-RR(L-DAB)SvP(L-DAB)PD |
| 442 | 606 | myr-RRHSTPHASE |
| 443 | 607 | myr-RRHSkPHPD |
| 444 | 608 | myr-RRHS(L-DAB)PHPD |
| 445 | 609 | myr-RRHS(L-DAB)PHAD |
| 446 | 610 | myr-RRHSEP(L-DAB)PD |
| 447 | 611 | myr-RR(L-DAB)SEPHPD |
| 448 | 622 | myr-RRHSvP(L-DAB)PD-NH2 |
| 449 | 624 | myr-RRHST(Aib)HAD |
| 450 | 630 | myr-RRHSTPHPDIEGR |
| 451 | 632 | myr-RRHSTPHPDIEGRGWQRPSSW |
| 452 | 633 | myr-RR(L-DAB)SEP(L-D AB)PD |
| 453 | 634 | myr-RRHSEP(L-DAB)PD-NH2 |
| 454 | 635 | myr-RR(L-DAB)SEPHPD |
| 455 | 636 | myr-RRHS(PSER)P(L-DAB)PD |
| 456 | 637 | myr-RRHS(pser)P(L-DAB)PD |
| 457 | 638 | myr-RRHS(PSER)P(L-DAB)PD-NH2 |
| 458 | 639 | myr-RRHSKP(L-DAB)PD |
| 459 | 640 | myr-RR(L-DAB)SKPHPD |
| 460 | 642 | myr-RRHSTPHPAH |
| 461 | 643 | myr-RRHSTPHPA(L-DAB) |
| 462 | 644 | myr-RRHSTPHPDH |
| 463 | 645 | myr-RRHSvP(L-DAB)PDH |
| 464 | 646 | myr-RRHSTPHADH |

myr stands for myristoyl group, Uppercase and lowercase letters stands for L-type and D-type amino acids respectively. L-DAB stands for L-type Diaminobutyric Acid. PSER and pser stand for L-type and D-type Phosphoserine, respectively. AIB stands for Aminoisobutyric acid.

The peptides were tested in anti-cancer assays on two cell lines. As can be seen in FIGS. 7-8 the indicated peptides are endowed with anti-cancer activity as determined by cell viability (crystal violet viability assay).

### Example 7

### NMR Experiments of pCAP-250-DBD Complex and its Peptide Variants

NMR experiments (1H-15N HSQC spectra) were performed in order to assess the structural effects that are induced by the binding of the pCAP-250 peptide (PCAP 250) to the p53 DBD. Since residue peak assignment was previously produced for WT DBD (94-312 of SEQ ID NO: 44) [Wong et al. supra], the NMR experiments were conducted using WT DBD (94-296, SEQ ID NO: 44), keeping the same conditions as described by Wong et al [supra].

FIG. 9 presents the NMR peak assignment obtained by Wong et al. (supra) together with the NMR peak map obtained for the free DBD and for the DBD-pCAP 250 complex. From FIG. 9 it can be seen that, in general, the map of Wong et al. (supra) was successfully reproduced despite the differences in the C-terminal lengths of the two DBD constructs, 296 versus 312. Many peak changes in a variety of intensities are observed between the maps of the free DBD and the DBD-pCAP 250, including the disappearing and emerging of a few unassigned peaks, thus clearly providing an indication for binding of pCAP 250 to the WT DBD. Mapping these changes on the DBD structure provides a clear picture regarding the three-dimensional structural region which is influenced by the binding of pCAP 250. This region mainly involves the helix-2 and the L1 loop of DBD-DNA interface motifs and it further extends into the central region of the protein (see magenta in FIG. 10). C277 and R280 are examples of moderate peak movements of residues located on helix-2, where the most dramatic peak movement is observed for G117, located on the L1 loop (see magenta and brown circles in FIG. 9).

Interestingly, the relatively low intensity peaks originally observed by Wong et al. (supra) for H115 and Y126 are not observed for the free DBD, but do appear upon the addition of the pCAP 250 peptide (see yellow circles in FIG. 10). Such a significant difference in the peaks assignment can be considered as the most dominant peak changes induced by pCAP 250. The low intensity of the original peaks and the absence of the peaks from the free DBD spectra indicate that these residues are located in a low stability structural region of the protein, which can adopt more than one dominant stable conformation, and thus is highly sensitive to small changes in protein conditions. Indeed, a dramatic structural reorganization is shown for H115 and Y126 when comparing the top two low energy conformations of a DBD structure solved by NMR (pdb code 2FEJ). Notably, the three-dimensional organization of H115 and Y126 is in close proximity to G117 and can directly affect it, and together these three residues are highly related to the structural integrity of the L1 loop (see FIGS. 11A-B). The appearance of the H115 and Y126 peaks upon peptide addition was further validated by additional NMR experiment using a different pCAP 250 peptide variant, pCAP-615 (RRHSTP{DAB}PD), SEQ ID NO: 465 (see FIG. 12).

The pCAP-553 (myr-RRHSvP(L-DAB)PD, v stands for D-type valine, SEQ ID NO: 429) pCAP 250 peptide variant was found to be two times more potent than P-250 in SW-480 cell-based assays harbouring mutant p53R273H (see FIG. 7). The NMR analysis indicates that pCAP-553 (P553) tends to bind the DBD with improved affinity. This is primarily reflected by the emergence of seven different novel and very strong unassigned peaks at the NMR peak map produced for the DBD-pCAP 553 complex in comparison to the free DBD. Additionally, the shapes of the peaks obtained for the DBD-pCAP 553 complex tend to be more unified and circular, indicating that the binding of the P553 peptide improves the structural stability of the DBD (see FIG. 13).

The NMR experimental results provide evidence for the explicit binding of pCAP 250 and its peptide variants to the WT DBD of the p53 protein. These results support the findings regarding the binding of pCAP 250 to the DBD using the MST methodology (FIGS. 3A-K). The NMR results further indicate that the binding of pCAP 250 and its peptide variants induces structural changes in the DBD, which directly influence the integrity and stability of the DBD-DNA binding interface region, namely the Helix-2 and the L1 loop structural motifs which are essential for the ability of the DBD to bind the DNA. The binding of pCAP 250 and its peptide variants further affects additional residues at the surroundings of the helix 2 and the L1 loop structural motifs, creating a relatively large yet decisive affected patch on the DBD surface.

### Example 8

### In Vivo Efficacy Study of pCAP-553 in the Treatment of Subcutaneous SU-DHL-8 Lymphoma Xenograft Model in Female CB17/SCID Mice

The objective of this study was to evaluate the in vivo therapeutic efficacy of pCAP-553 in the treatment of the subcutaneous SU-DHL-8 lymphoma xenograft model in female CB17/SCID.

### Experimental Methods

Cell culture—Cell line SU-DHL was grown in RPMI1640 medium (10% FBS) at 37° C., 5% CO₂ in air.

Tumor inoculation—Each mouse was inoculated subcutaneously in the right upper flank region with tumor cells (5×10⁶) in 0.1 ml of PBS mixed with matrigel (1:1) for tumor development.

Randomization—The randomization started when the mean tumor size reached approximately 90.22 mm³. A total of 60 mice were enrolled in the study and randomly allocated to 6 study groups, with 10 mice per group. Randomization was performed based on “Matched distribution” method (Study Director™ software, version 3.1.399.19). The date of randomization was denoted as day 0.

Observation and Data Collection—After tumor inoculation, the animals were checked daily for morbidity and mortality. During routine monitoring, the animals were checked for any effects of tumor growth and treatments on behavior such as mobility, food and water consumption, body weight gain/loss (Body weights would be measured three times per week after randomization), eye/hair matting and any other abnormalities. Mortality and observed clinical signs were recorded for individual animals in detail.

Tumor volumes were measured three times per week after randomization in two dimensions using a caliper, and the volume was expressed in mm³ using the formula: V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet. The body weights and tumor volumes were measured by using Study Director™software (version 3.1.399.19).

TABLE 4
Drug formulation

| | Conc. | Dose | Formulation Details and | Physical | |
|---|---|---|---|---|---|
| Drug | (mg/mL) | (mg/kg) | Preparation Frequency | Description | Storage |
| pCAP 553 | 20 or 30 | 100 or 150 | 20% PEG400 in 50 mM PBS | Solution | — |
| | | | pH = 5, freshly prepared | | |
| Positive control | 1 or 1.5 | 10 or 15 | prepared as a solution in NaCl | Solution | In solvent: −80° C., |
| BAY 1238097 | | | 0.9% in water, pH 4 | | 6 months |
| Vehicle | — | — | saline | Solution | — |

Test Article Administration—The treatment was initiated on day 0 post randomization.

Study endpoint—Tumor growth inhibition (TGI): TGI % is an indication of antitumor activity, and expressed as: TGI (%)=100×(1-T/C). T and C are the mean tumor volume (or weight) of the treated and control groups, respectively, on a given day. The treatment had been performed for 11 days.

Body weight loss (BWL)—The body weight of all animals was monitored throughout the study and animals would be euthanized if they lost over 20% of their body weight relative to the weight at the first day of treatment.

Tumor size—Individual mouse was sacrificed when tumor volume exceeding 3000 mm³.

Statistical analysis—To compare tumor volumes of different groups at a pre-specified day, Bartlett's test was used to check the assumption of homogeneity of variance across all groups. When the p-value of Bartlett's test was >=0.05, one-way ANOVA was run to test overall equality of means across all groups. If the p-value of the one-way ANOVA was <0.05, Dunnett's tests was further performed for comparing each treatment group with the vehicle group. When the p-value of Bartlett's test was <0.05, Kruskal-Wallis test was run to test overall equality of medians among all groups. If the p-value the Kruskal-Wallis test was <0.05, post hoc testing was performed by running Conover's non-parametric test for all pairwise comparisons or for comparing each treatment group with the vehicle group, both with single-step p-value adjustment.

All statistical analyses had been done in R-a language and environment for statistical computing and graphics (version 3.3.1). All tests were two-sided unless otherwise specified, and p-values of <0.05 were regarded as statistically significant.

Results:

The therapeutic effect of pCAP553 was examined either alone or in a combination with BAY1238097. BAY1238097 is an inhibitor of the Bromodomain (BRD) and Extra-Terminal domain (BET) family of proteins. Upon administration, the BET inhibitor BAY1238097 binds to the acetylated lysine recognition motifs on the BRD of BET proteins, thereby preventing the interaction between BET proteins and histones. This disrupts chromatin remodeling and prevents the expression of certain growth-promoting genes, leading to cancer cell death and inhibition of tumor growth.

In efficacy study, according to the mortality and tolerability observation, no signs of cachexia were observed in vehicle group (Group 1). In addition, no more than 20% BWL was observed in the peptide treatment groups as well as the combination treatment group (pCAP553, 100 mg/kg and BAY1238097, 10 mg/kg). The body weight change curves of mice in each group at different time points are shown in FIGS. 15A-B.

The therapeutic efficacy of test peptides pCAP553 (100 and 150 mg/kg) as single agents, pCAP553 100 mg/kg and BAY1238097 10 mg/kg as a combination treatment were evaluated in SU-DHL-8 subcutaneous human lymphoma cancer xenograft model in female CB17/SCID mice. All peptide treatment groups including the combination treatment group produced significant antitumor efficacy in the SU-DHL-8 model. To keep integrality of data, the statistical analysis was performed with the data collected from day 7. Significant anti-tumor activity was observed in the pCAP553 (100 mg/kg), pCAP553 (150 mg/kg), and pCAP553 combined with BAY1238097, where the calculated TGI was at 61.69% (p<0.001), 70.72% (p<0.001) and 75.71% (p<0.001) respectively, and significant anti-tumor effect was also produced by BAY1238097, positive control groups (p<0.001). The tumor growth curves are illustrated in FIG. 16. Tumor growth % inhibition values are presented in Table 5:

TABLE 5
Tumor growth inhibition compared with negative control Group 1

| Group | Day 0 | Day 2 | Day 4 | Day 7 | Day 9 | Day 11 |
|---|---|---|---|---|---|---|
| G01 | — | — | — | — | — | — |
| G02 | 0.00% | 34.85% | 52.05% | 61.69% | 54.82% | 39.88% |
| G03 | 0.01% | 40.65% | 63.55% | 70.72% | 68.18% | 49.36% |
| G04 | 0.01% | 50.28% | 58.46% | 53.97% | 53.94% | 46.39% |
| G05 | 0.02% | 42.41% | 57.91% | 61.47% | 63.48% | 59.76% |
| G06 | 0.01% | 55.39% | 71.01% | 75.71% | 73.94% | 68.18% |

The combination effect of different concentrations of pCAP-553 and BAY1238097 was further examined in cell viability assays. As shown in FIG. 17, high levels of BAY1238097 (0.25-5 μM) cause almost complete cell death, whereas low BAY1238097 concentrations (0.01-0.07 μM) have very little effect on cell viability. The combined treatment of pCAP-553 together with low to intermediate concentrations of BAY1238097 (0.02-0.1 μM) shows that IC50 was reduced from 0.5 μM for pCAP-553 alone to about 150 nM for the combination with 0.06 μM BAY1238097. Advantageously, the peptide enables the use of reduced amounts of BAY1238097 and accordingly, may reduce possible side effects and toxicity.

### Example 9

### In Vitro Efficacy Study of pCAP-250 in the Treatment of Multiple Myeloma in MM Cell Lines

To test the effect of pCAP-250 on multiple myeloma (MM), a model of MM cell lines was used. FIG. 18 shows the response of different multiple myeloma cell lines to treatment with either lead peptide pCAP-250 (FIG. 18A) or the control scrambled sequence pCAP-704 (FIG. 18B). The p53 status of the different cell lines is as follows: KMS11 is p53-null, MM.1S and H929 express wtp53, KMS20 expresses a nonsense p53 mutation resulting in a truncated protein, whereas U266 and OPM2 express two different p53 missense mutations (A161T and R175H, respectively). As seen in the figure, there is a correlation between the status of p53 protein and the response to pCAP-250. The two lines with missense mutations are the most sensitive to treatment with pCAP-250 (IC50 of 1.5 μM-3 μM), presumably due to the reactivation of the mutp53 and induction of programed cell death. The two lines that express wtp53 exhibit an intermediate response (IC50 of 12 μM-18 μM). KMS11 (deleted for p53 expression) and KMS20, expressing p53 that is lacking the DNA binding domain, show the lowest sensitivity to pCAP-250 (IC50>40 μM).

FIG. 19 shows the response of multiple myeloma cell lines to treatments with combinations of the peptide pCAP-250 and BAY1238097. As seen in FIG. 19, high levels of BAY1238097 (0.25-5 μM) cause almost complete cell death, whereas low BAY1238097 concentrations (0.01-0.07 μM) have very little effect on cell viability. The combined treatment of pCAP-250 together with low to intermediate concentrations of BAY1238097 (0.02-0.1 μM) shows an additive effect, lowering the IC50 by 2-3 folds (from 2.5 μM for pCAP-250 alone to about 1 μM for the combination) in OPM2 cells.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of treating a hematologic cancer associated with a mutant p53 protein, comprising administering to a subject in need thereof a therapeutically effective amount of an isolated peptide comprising an amino acid sequence selected from the group consisting of SEQ ID NOs. 1, 426, 427, 429, 430, 431, 443, 446, 448, 449, 453, 457, 458 and 462.

2. The method of claim 1, wherein said peptide at least partially reactivates a mutant p53 protein and induces the mutant p53 protein to exhibit p53-selective inhibition of cancer cells.

3. The method of claim 1, wherein the hematologic cancer is lymphoma.

4. The method of claim 3, wherein the lymphoma is non-Hodgkin lymphoma or Hodgkin lymphoma.

5. The method of claim 1, wherein the hematologic cancer is multiple myeloma.

6. The method of claim 1, wherein the isolated peptide having at least one arginine residue attached to the N-terminal amino acid of said amino acid sequence and an aspartic acid residue attached to the C-terminal amino acid of said amino acid sequence.

7. The method of claim 1, wherein the isolated peptide comprising at least one additional amino acid attached to the C-terminus of said amino acid sequence.

8. The method of claim 1, wherein said peptide binds to p53 protein via the p53 consensus DNA binding element comprising the nucleic acid sequences set forth in SEQ ID NO: 55 and 56).

9. The method of claim 1, wherein the isolate peptide consisting of the amino acid sequence set forth in SEQ ID NO: 429.

10. The method of claim 1, wherein the isolate peptide consisting of the amino acid sequence set forth in SEQ ID NO: 1.

11. The method of claim 1, wherein the isolated peptide further comprising a cell penetrating moiety.

12. The method of claim 1, wherein said peptide binds to p53 protein via the p53 consensus DNA binding element comprising the nucleic acid sequences set forth in SEQ ID NO: 55 and 56.

13. The method of claim 1, further comprising administering to the subject in need thereof a therapeutically effective amount of an inhibitor of Bromodomain (BRD) and Extra-Terminal domain (BET) family.

14. The method of claim 13, wherein the inhibitor is Bay1238097.

15. The method of claim 1, further comprising administering to the subject in need thereof a therapeutically effective amount of a platin-based chemotherapy.

16. The method of claim 1, wherein said therapeutically effective amount of the isolated peptide is 0.01-0.3 mg/kg per day.
